(19) United States
(12) Patent Application Publication
(10) Pub. No.: US 2016/0015759 A1
(43) Pub. Date: Jan. 21, 2016

(54) COMPOSITIONS AND METHODS FOR TREATING AN IMMUNODEFICIENCY VIRUS INFECTION

(71) Applicant: THE J. DAVID GLADSTONE INSTITUTES, San Francisco, CA (US)

(72) Inventors: Leor S. Weinberger, San Francisco, CA (US); Timothy J. Notton, San Francisco, CA (US)

(21) Appl. No.: 14/769,025

(22) PCT Filed: Mar. 13, 2014
(86) PCT No.: PCT/US14/26423

§ 371 Date: Aug. 19, 2015

Related U.S. Application Data
(60) Provisional application No. 61/784,844, filed on Mar. 14, 2013.

(51) Int. Cl. A61K 35/76; C12N 7/00

(52) U.S. Cl. CPC A61K 35/76; C12N 7/00; C12N 2740/16032; C12N 2740/16021

(57) ABSTRACT

The present disclosure provides an interfering, conditionally replicating human immunodeficiency virus (HIV) construct; infectious particles comprising the constructs; and compositions comprising the construct or the particle. The constructs, particles, and compositions are useful in methods of reducing HIV viral load in an individual, which methods are also provided.

### CROSS-REFERENCE

This application claims the benefit of U.S. Provisional Patent Application No. 61/784,844, filed Mar. 14, 2013, which application is incorporated herein by reference in its entirety.

### STATEMENT REGARDING FEDERALLY SPONSORED RESEARCH

This invention was made with government support under Grant No. OD006677 awarded by the National Institutes of Health. The government has certain rights in the invention.

### INCORPORATION BY REFERENCE OF SEQUENCE LISTING PROVIDED AS A TEXT FILE

A Sequence Listing is provided herewith as a text file, “GLAD-404WO_SeqList_ST25.txt” created on Mar. 9, 2014 and having a size of 30 KB. The contents of the text file are incorporated by reference herein in their entirety.

### INTRODUCTION

Defective interfering particles (DIPs) are mutant versions of viruses that contain significant genomic deletions such that they are unable to replicate except when complemented by wild-type virus replicating within the same cell. At the most fundamental level, DIPs arise because viral genomes encode both cis- and trans-acting elements. Trans-acting elements (trans-elements) code for gene products, such as capsid proteins or transcription factors, and cis-acting elements (cis-elements) are regions of the viral genome that interact with trans-element products to achieve productive viral replication including viral genome amplification, encapsidation, and viral egress. In the case of RNA virus genomes, cis elements can include viral enhancers and promoters, and also viral genome packaging signals. Viral capsid and envelope proteins, on the other hand, are examples of trans elements. Mutations that result in loss of at least one obligate trans-element but retain all necessary cis-elements required for productive replication can generate DIPs.

### LITERATURE

U.S. Pat. No. 7,572,906; Metzger et al. (2011) PLoS Comp. Biol. 7:e1002015; Dull et al. (1998) J. Virol. 72:8463-71; Huang and Baltimore (1970) Nature 226:325-7; Levine et al. (2006) Proc. Natl. Acad. Sci. USA 103:17372-7; Weinberger et al. (2003) J. Virol. 77:10028-36

### SUMMARY

The present disclosure provides an interfering, conditionally replicating human immunodeficiency virus (HIV) construct; infectious particles comprising the construct; and compositions comprising the construct or the particle. The constructs, particles, and compositions are useful in methods of reducing HIV viral load in an individual, which methods are also provided.

### BRIEF DESCRIPTION OF THE DRAWINGS

FIGS. 1A-D. Divergent evolution of the HIV-1 and DIP dimerization initiation sequences by double mutations in HIV-1 implies that DIP interference by “genome-stealing” is evolutionary unstable.

FIGS. 2A-E. DIPs that steal capsid stably suppress HIV-1 load across a broad range of parameters.

FIGS. 3A-E. DIP-HIV interaction is evolutionary stable over a broad parameter range: HIV-1 cannot escape DIP by decreasing packaging constant.

FIGS. 4A-E. DIV-HIV interaction is evolutionary stable over a broad parameter range: HIV-1 cannot escape DIP by decreasing the capsid-to-genome ratio.

FIG. 5 provides a plot of evolution in a dimerization initiation sequence. Dimerization coefficients for HIV-HIV, HIV-DIP and DIP-DIP (defined in Equations in FIG. 1b) are shown qualitatively versus mutation pair number.

FIG. 6 provides curves showing DIP contribution to suppression of HIV-1 viral load. The curves show the ratio of HIV load to its value in the absence of DIP (P=0). Values of η and P are shown.

FIGS. 7A-B provides curves depicting (A) the relationships where average number of DIP provirus copies per cell is shown as a function of waste parameter κ; at several values of η: η=2 (red), η=5 (green), η=10 (blue); and (B) steady-state HIV-1 load when multiplicity of DIP infection, m, is restricted to ≦1.

FIG. 8 provides in vivo estimates for the relationship of waste parameter κ and capsid-to-genome production η.

FIG. 9 presents whole-cell reverse transcription-quantitative polymerase chain reaction (RT-qPCR) analysis of co-treatment of TIP with HIV, where the data are normalized to ActB reference.

FIG. 10 presents whole-cell RT-qPCR analysis of co-treatment of TIP with HIV, where the data are normalized to PPIA reference.

FIG. 11 presents cell-free viral supernatant RT-qPCR analysis of co-treatment of TIP with HIV.

FIG. 12 presents flow cytometry results of HIV-1 (NL4-3G) and TIP (SR2-D1) co-infection.

FIG. 13 presents results (infectious Titer on MT4 cells) from co-transfection experiments with various TIPs and HIV.

FIG. 14 provides the intracellular expression of various genes as quantified by qRT-PCR (the data was normalized over expression of ActB) for various co-transfection experiments.

FIG. 15 provides results from FACs plot data (see FIG. 16) showing the % positive MT4s cells from infections. HEK-293T cells were co-transfected with various constructs, and various dilutions of the resulting supernatants were used to infect naïve MT4s cells.

FIGS. 16A-F provide FACs plots showing the % positive MT4s cells from infections. HEK-293T cells were co-transfected with various constructs, and various dilutions of the resulting supernatants were used to infect naïve MT4s cells. Y-axis is from BFP detection (TIP) and X-axis is GFP detection (HIV). (FIG. 16 presents the flow cytometry data summarized in FIG. 13)

FIG. 17 provides vector maps of (A) HIV-1 (NL4-3) (SEQ ID NO: 1); (B) TIP (SR2-D1) (SEQ ID NO: 2); and (C) TIP (SR2-D1-delEF1a-delmTagBFP2) (SEQ ID NO: 19).

FIGS. 18A-N provide a comparison of the TIP (SR2-D1) (SEQ ID NO: 2) and HIV-1 (NL4-3) (SEQ ID NO: 1) nucleotide sequences.

FIGS. 19A-D provide an annotated nucleotide sequence of TIP (SR2-D1-delEF1a-delmTagBFP2) (SEQ ID NO: 19).

### DEFINITIONS

The term “immunodeficiency virus” includes human immunodeficiency virus (HIV), feline immunodeficiency virus, and simian immunodeficiency virus. The term “human immunodeficiency virus” as used herein, refers to human immunodeficiency virus-1 (HIV-1); human immunodeficiency virus-2 (HIV-2); and any of a variety of HIV subtypes and quasispecies.

As referred to herein, a “pseudotype envelope” is an envelope protein other than the one that naturally occurs with the retroviral core virion, which encapsidates the retroviral core virion (resulting in a phenotypically mixed virus).

A “virus” is an infectious agent that consists of protein and nucleic acid, and that uses a host cell's genetic machinery to produce viral products specified by the viral nucleic acid. A “nucleic acid” refers to a polymer of DNA or RNA that is single or double-stranded, linear or circular, and, optionally, contains synthetic, nonnatural, or modified nucleotides, which are capable of being incorporated into DNA or RNA polymers. A DNA polynucleotide preferably is comprised of genomic or cDNA sequences.

A “wild-type strain of a virus” is a strain that does not comprise any of the human-made mutations as described herein, i.e., a wild-type virus is any virus that can be isolated from nature (e.g., from a human infected with the virus). A wild-type virus can be cultured in a laboratory, but still, in the absence of any other virus, is capable of producing progeny genomes or virions like those isolated from nature.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of an agent (e.g., a construct, a particle, etc., as described herein) that, when administered to a mammal (e.g., a human) or other subject for treating a disease, is sufficient to effect such treatment for the disease. The “therapeutically effective amount” can vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “pharmaceutical composition” is meant to encompass a composition suitable for administration to a subject, such as a mammal, e.g., a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an interfering particle” includes a plurality of such particles and reference to “the cis-acting element” includes reference to one or more cis-acting elements and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides an interfering, conditionally replicating human immunodeficiency virus (HIV) construct; infectious particles comprising the construct; and compositions comprising the construct or the particle. The constructs, particles, and compositions are useful in methods of reducing HIV viral load in an individual, which methods are also provided.

### Interfering Constructs

The present disclosure provides an interfering, conditionally replicating human immunodeficiency virus (HIV) construct. For simplicity, the interfering, conditionally replicating HIV constructs are referred to as “interfering constructs” or “TIPs.” A subject interfering construct is conditionally replicating, e.g., a subject interfering construct, when present in a mammalian host, cannot, in the absence of a wild-type HIV, form infectious particles containing copies of itself. A subject interfering construct can be packaged into an infectious particle in vitro in a laboratory (e.g., in an in vitro cell culture) when the appropriate polypeptides required for packaging are provided. The infectious particle can deliver the interfering construct into a host cell, e.g., an in vivo host cell. Once inside an in vivo host cell (a host cell in a mammalian subject), the interfering construct can integrate into the genome of the host cell, or can remain cytoplasmic. The interfering construct can replicate in the in vivo host cell only in the presence of a wildtype HIV. When an in vivo host cell comprising an interfering construct is infected by a wildtype HIV, the interfering construct replicates (e.g., is transcribed and packaged) substantially more efficiently than the wildtype HIV, thereby outcompeting the wildtype HIV. As a result, the HIV viral load is substantially reduced in the individual.

An interfering construct of the present disclosure can be an RNA construct, or a DNA construct (e.g., a DNA copy of an RNA).

An interfering construct of the present disclosure does not include any heterologous nucleotide sequences, e.g., sequences not derived from HIV. An interfering construct of the present disclosure does not include any heterologous nucleotide sequences that encode a gene product. Gene products include polypeptides and RNA. “Heterologous” refers to a nucleotide sequence that is not normally present in a wild-type HIV in nature.

A subject interfering construct comprises HIV cis-acting elements; and comprises an alteration in the HIV nucleotide sequence such that alteration renders one or more encoded HIV trans-acting polypeptides non-functional. By “non-functional” is meant that the HIV trans-activating polypeptide does not carry out its normal function, due to truncation of or internal deletion within the encoded polypeptide, or due to lack of the polypeptide altogether. “Alteration” of an HIV nucleotide sequence includes: deletion of one or more nucleotides and/or substitution of one or more nucleotides.

In some cases, a subject interfering construct, when present in a host cell (e.g., in a host cell in an individual) that is infected with a wildtype HIV, replicates at a rate that is at least about 10%, at least about 20%, at least about 30%, at least about 40%, at least about 50%, at least about 75%, at least about 2-fold, at least about 2.5-fold, at least about 5-fold, at least about 10-fold, or greater than 10-fold, higher than the rate of replication of the wildtype HIV in a host cell of the same type that does not comprise a subject interfering construct.

In some cases, a subject interfering construct, when present in a host cell (e.g., in a host cell in an individual) that is infected with a wildtype HIV, reduces the amount of wildtype HIV transcripts in the cell by at least about 20%, at least about 30%, at least about 40%, at least about 50%, at least about 60%, at least about 70%, at least about 80%, or at least about 90%, compared to the amount of wildtype HIV transcripts in a host cell that is infected with wildtype HIV, but does not comprise a subject interfering construct.

In some cases, a subject interfering construct, when present in a host cell (e.g., in a host cell in an individual) that is infected with a wildtype HIV, results in production of interfering construct-encoded RNA such that the ratio (by weight, e.g., μg:μg) of interfering construct-encoded RNA to wild-type HIV-encoded RNA in the cytoplasm of the host cell is greater than 1. In some cases, a subject interfering construct, when present in a host cell (e.g., in a host cell in an individual) that is infected with a wildtype HIV, results in production of interfering construct-encoded RNA such that the ratio (by weight, e.g., μg:μg) of interfering construct-encoded RNA to wild-type HIV-encoded RNA in the cytoplasm of the host cell is from at least about 1.5:1 to at least about 10²:1 or greater than 10²:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

In some cases, a subject interfering construct, when present in a host cell (e.g., in a host cell in an individual) that is infected with a wildtype HIV, results in production of interfering construct-encoded RNA such that the ratio (e.g., molar ratio) of interfering construct-encoded RNA to wild-type HIV-encoded RNA in the cytoplasm of the host cell is greater than 1. In some cases, a subject interfering construct, when present in a host cell (e.g., in a host cell in an individual) that is infected with a wildtype HIV, results in production of interfering construct-encoded RNA such that the ratio (e.g., molar ratio) of interfering construct-encoded RNA to wild-type HIV-encoded RNA in the cytoplasm of the host cell is from at least about 1.5:1 to at least about 10²:1 or greater than 10²:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

Any convenient method can be used to measure the ratio of interfering construct-encoded RNA to wild-type HIV-encoded RNA in the cytoplasm of the host cell. Suitable methods can include, for example, measuring transcript number directly via qRT-PCR of both an interfering construct-encoded RNA and a wild-type HIV-encoded RNA; measuring levels of a protein encoded by the interfering construct-encoded RNA and the wild-type HIV-encoded RNA (e.g., via western blot, ELISA, mass spectrometry, etc.); and measuring levels of a detectable label associated with the interfering construct-encoded RNA and the wild-type HIV-encoded RNA (e.g., fluorescence of a fluorescent protein that is encoded by the RNA and is fused to a protein that is translated from the RNA). Such measurements can be performed, for example, after co-transfection experiments described in Examples 2 and 3 of the working examples below, using any convenient cell type.

In some embodiments, the interfering construct-encoded RNA is packaged. In some embodiments, the interfering construct-encoded RNA is unpackaged. In some cases, the interfering construct-encoded RNA includes both packaged and unpackaged RNA.

In some cases, a subject interfering construct, when present in a host cell (e.g., in a host cell in an individual) that is infected with a wildtype HIV, dimerizes with wildtype gRNA HIV genomes. In some cases, a subject interfering construct, when present in a host cell (e.g., in a host cell in an individual) that is infected with a wildtype HIV, dimerizes with a wildtype gRNA HIV genome and inhibits dimerization of wild-type HIV.

Both wildtype HIV and a subject interfering construct can be packaged into an infectious particle by a host cell (e.g., in a host cell in an individual). In some cases, where a host cell comprises both a subject interfering construct and a wildtype HIV, the ratio of interfering construct-containing particles produced by the host cell to wildtype HIV-containing particles is greater than 1. In some cases, where a host cell comprises both a subject interfering construct and a wildtype HIV, the ratio of interfering construct-containing particles produced by the host cell to wildtype HIV-containing particles is from at least about 1.5:1 to at least about 10²:1 or greater than 10²:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

A wildtype HIV genome is approximately 9700 nucleotides in length, e.g., from about 9700 nucleotides to about 9800 nucleotides in length). In contrast, a subject interfering construct has a genome that is some fraction of the total HIV genome length, such as from about 1000 nucleotides (nt) to about 9700 nt, e.g., from about 1000 nt to about 2000 nt, from about 2000 nt to about 3000 nt, from about 3000 nt to about 4000 nt, from about 4000 nt to about 5000 nt, from about 5000 nt to about 6000 nt, from about 6000 nt to about 7000 nt, from about 7000 nt to about 8000 nt, from about 8000 nt to about 9000 nt, or from about 9000 nt to about 9700 nt.

In some cases, a subject interfering construct has a length of from about 2500 nucleotides (nt) to about 4000 nt, e.g., from about 2500 nt to about 2600 nt, from about 2600 nt to about 2700 nt, from about 2700 nt to about 2800 nt, from about 2800 nt to about 2900 nt, from about 2900 nt to about 3000 nt, from about 3000 nt to about 3100 nt, from about 3100 nt to about 3200 nt, from about 3200 nt to about 3300 nt, from about 3300 nt to about 3400 nt, from about 3400 nt to about 3500 nt, from about 3500 nt to about 3600 nt, from about 3600 nt to about 3700 nt, from about 3700 nt to about 3800 nt, from about 3800 nt to about 3900 nt, or from about 3900 nt to about 4000 nt.

A subject interfering construct can exhibit a basic reproductive ratio (R₀) (also referred to as the “basic reproductive number”) that is greater than 1. R₀ is the number of cases one case generates on average over the course of its infectious period. When R₀ is 1, the infection will be able to spread in a population. Thus, a subject interfering construct has the capacity to spread from one individual to another in a population. In some cases, subject interfering construct (or a subject interfering particle) has an R₀ from about 2 to about 5, from about 5 to about 7, from about 7 to about 10, from about 10 to about 15, or greater than 15.

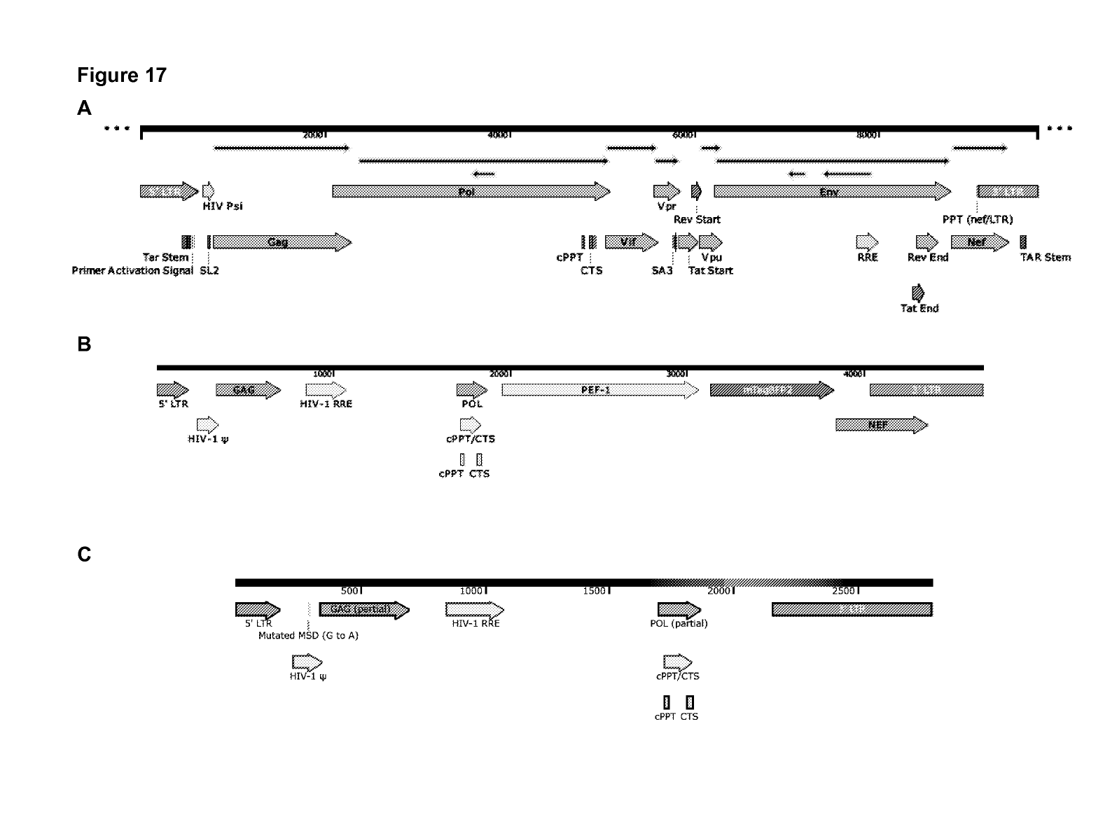

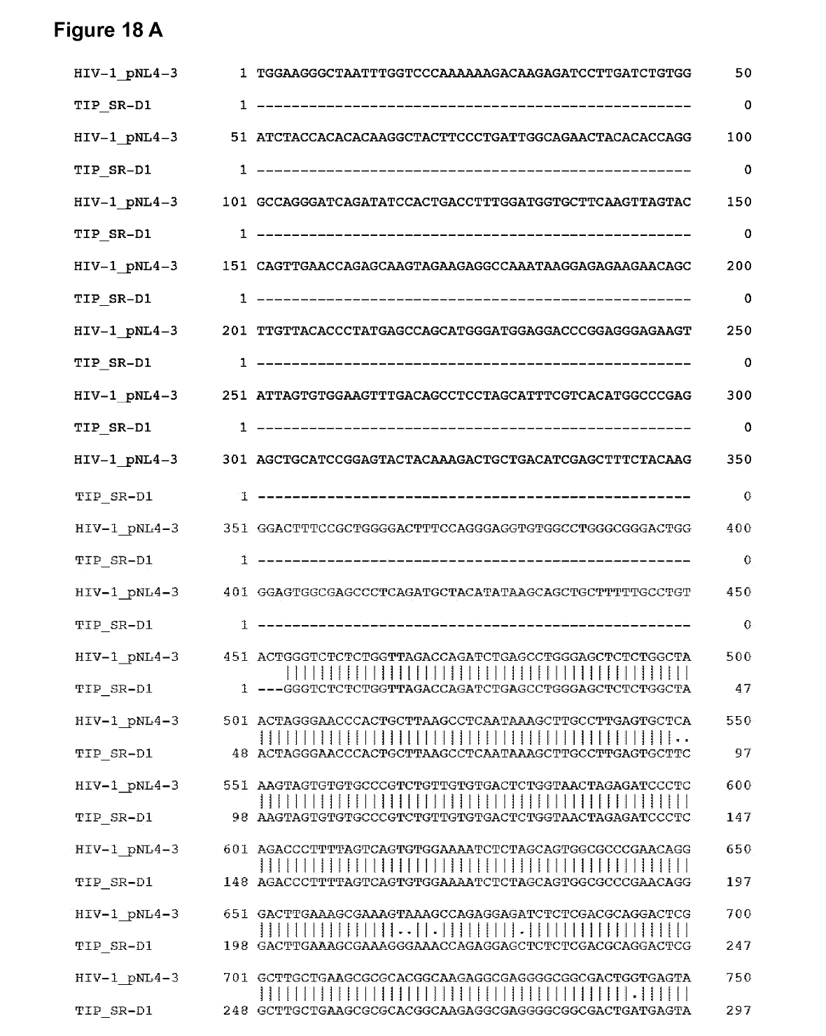

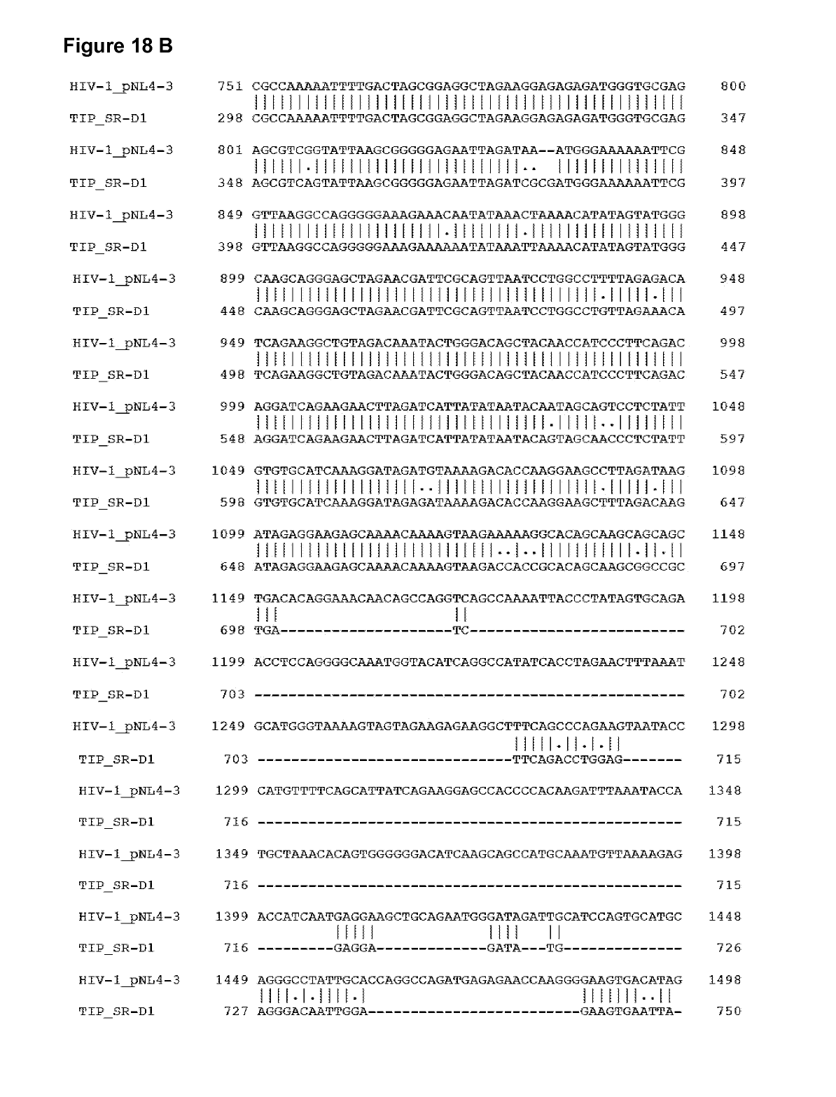

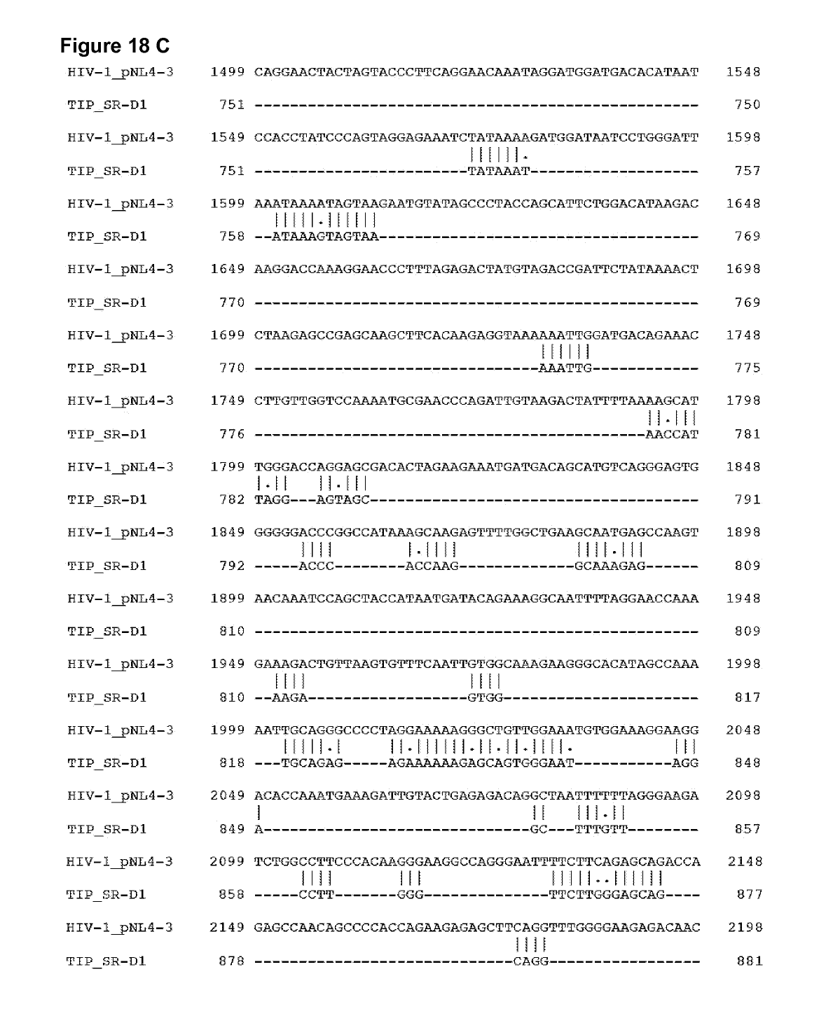

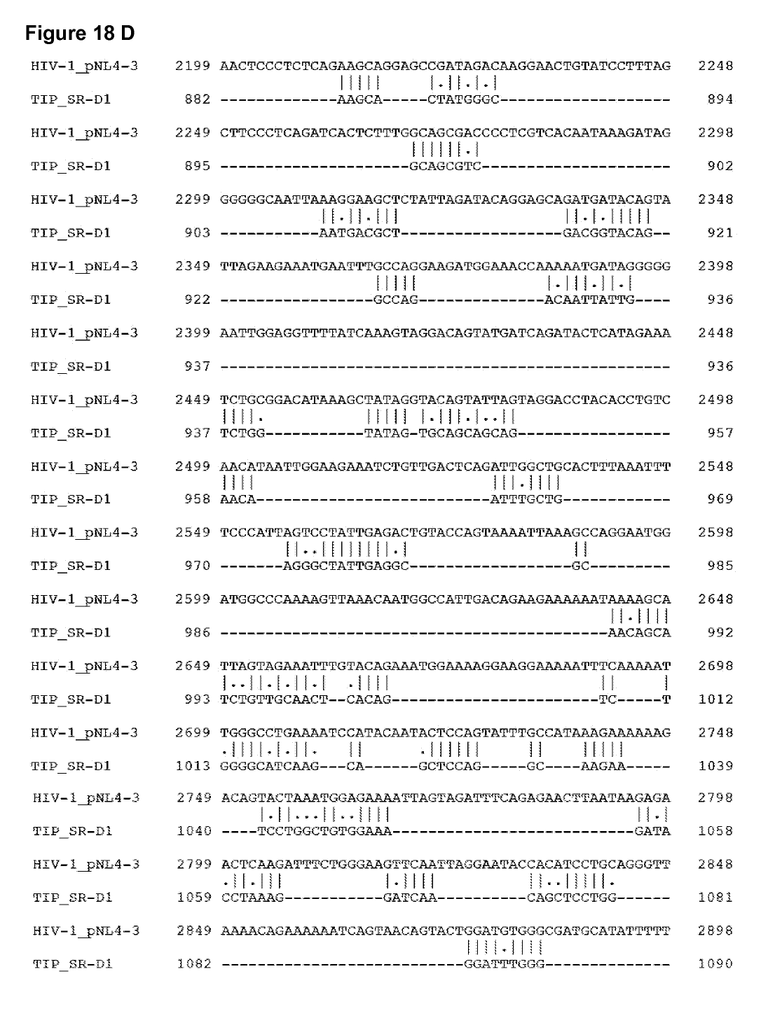

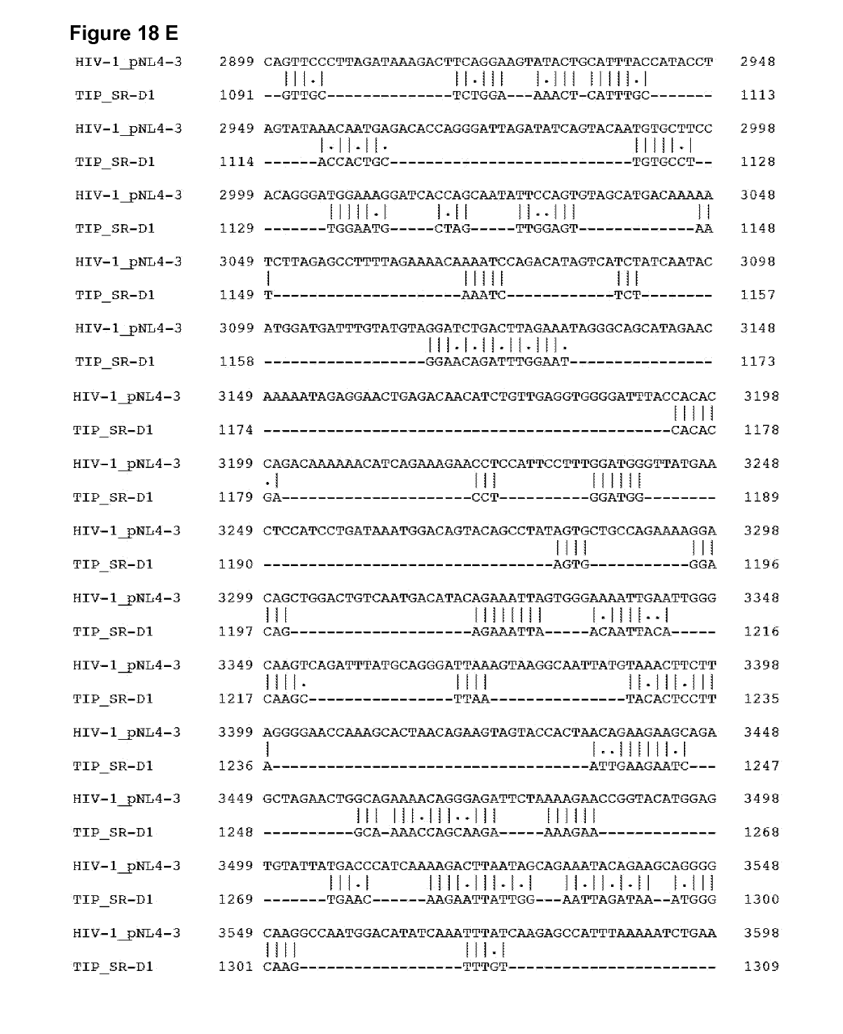

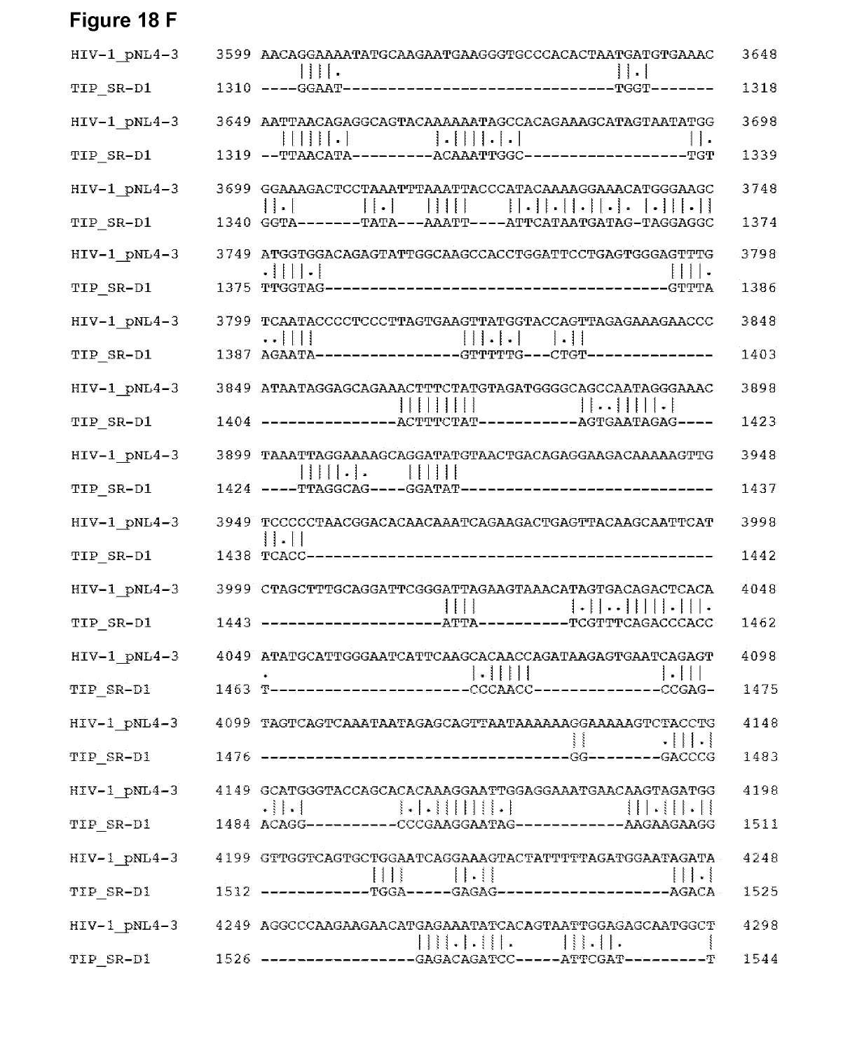

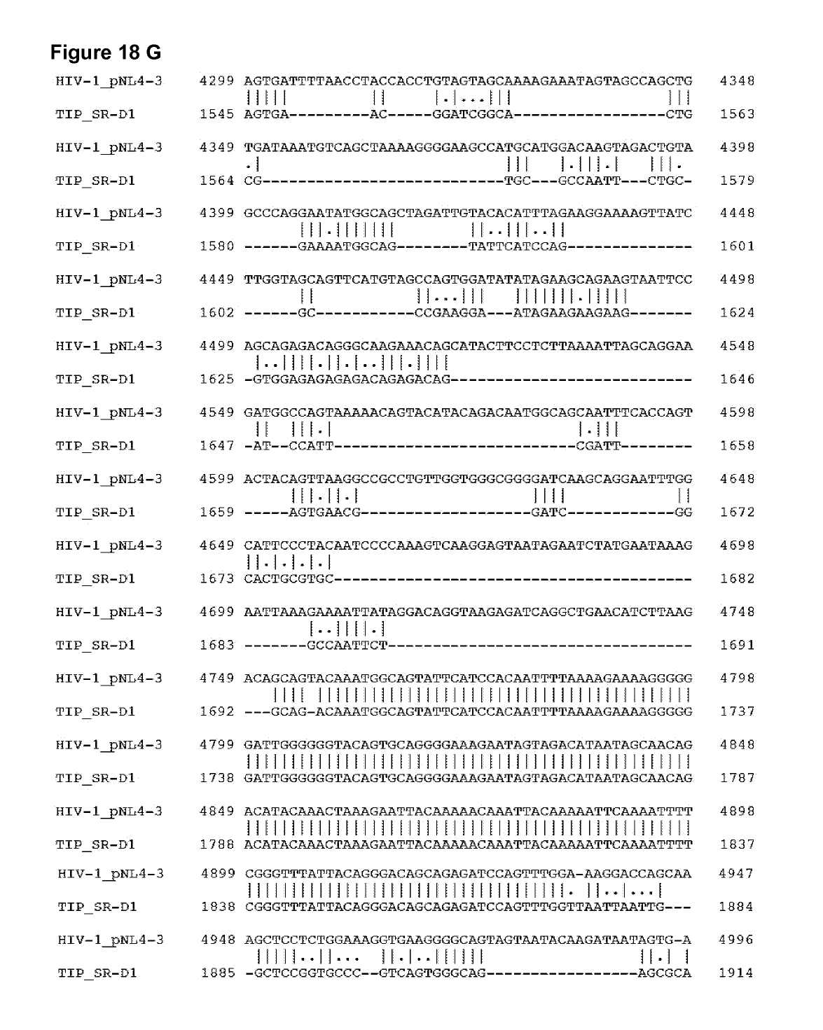

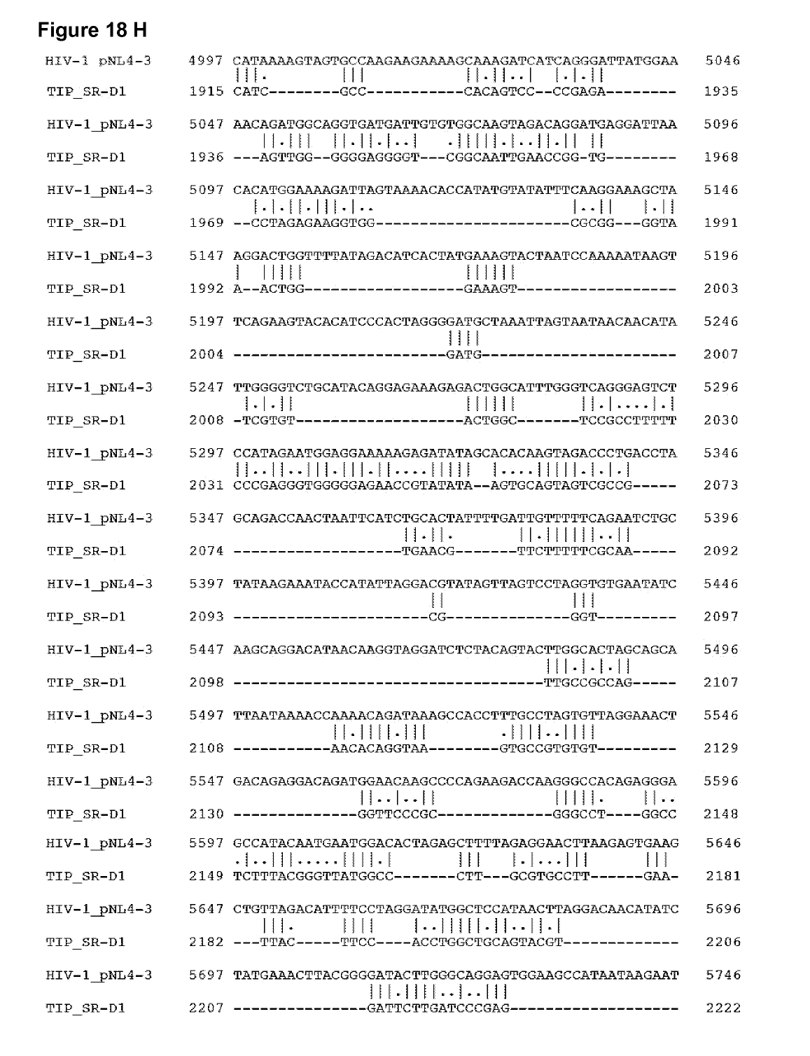

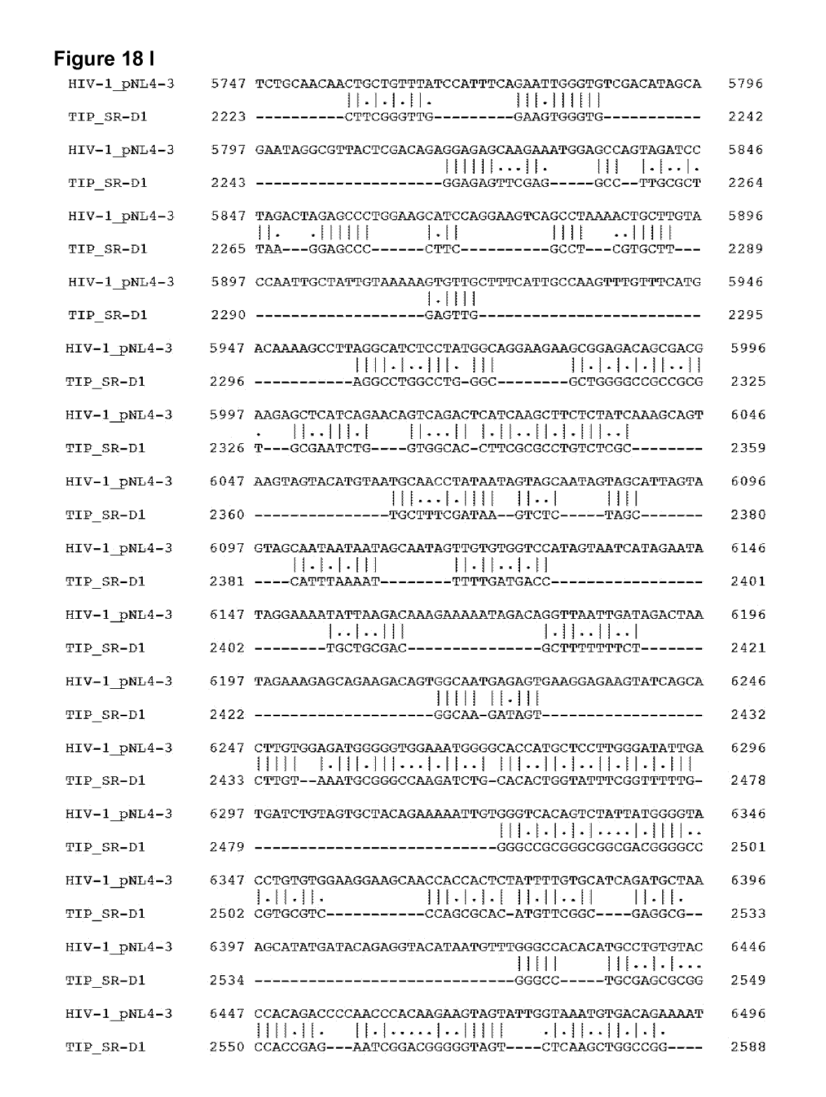

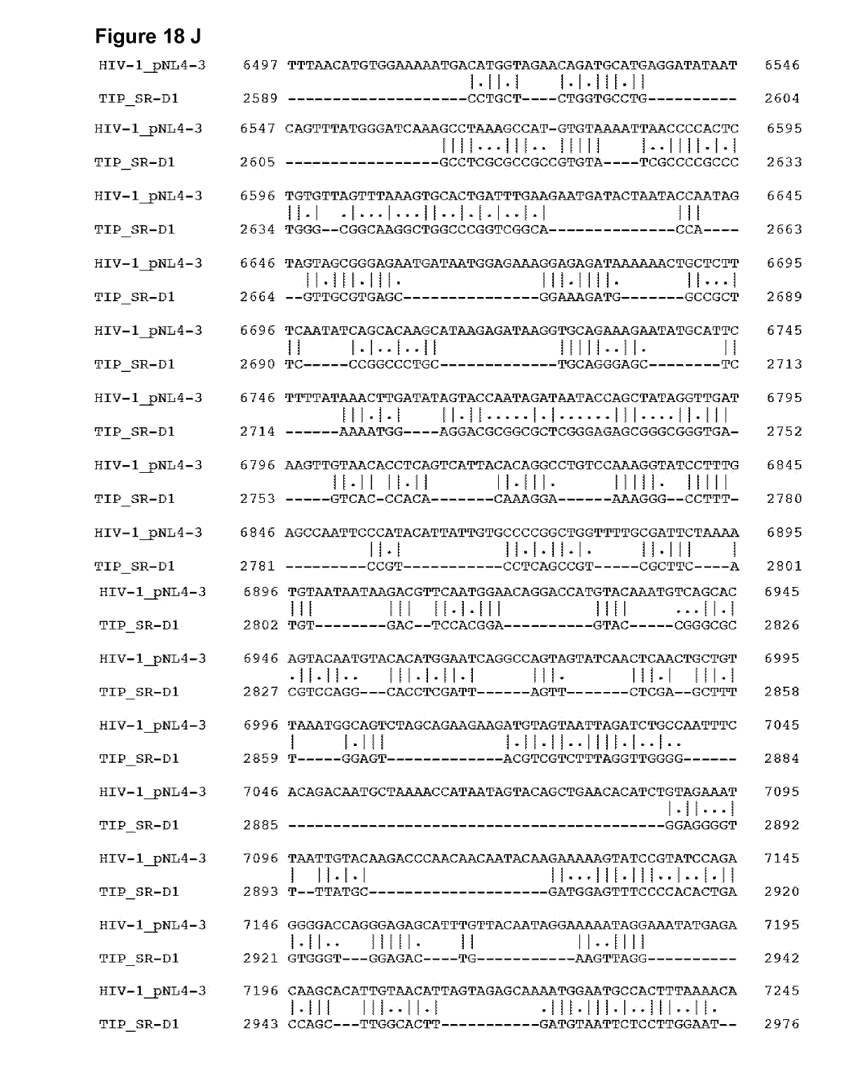

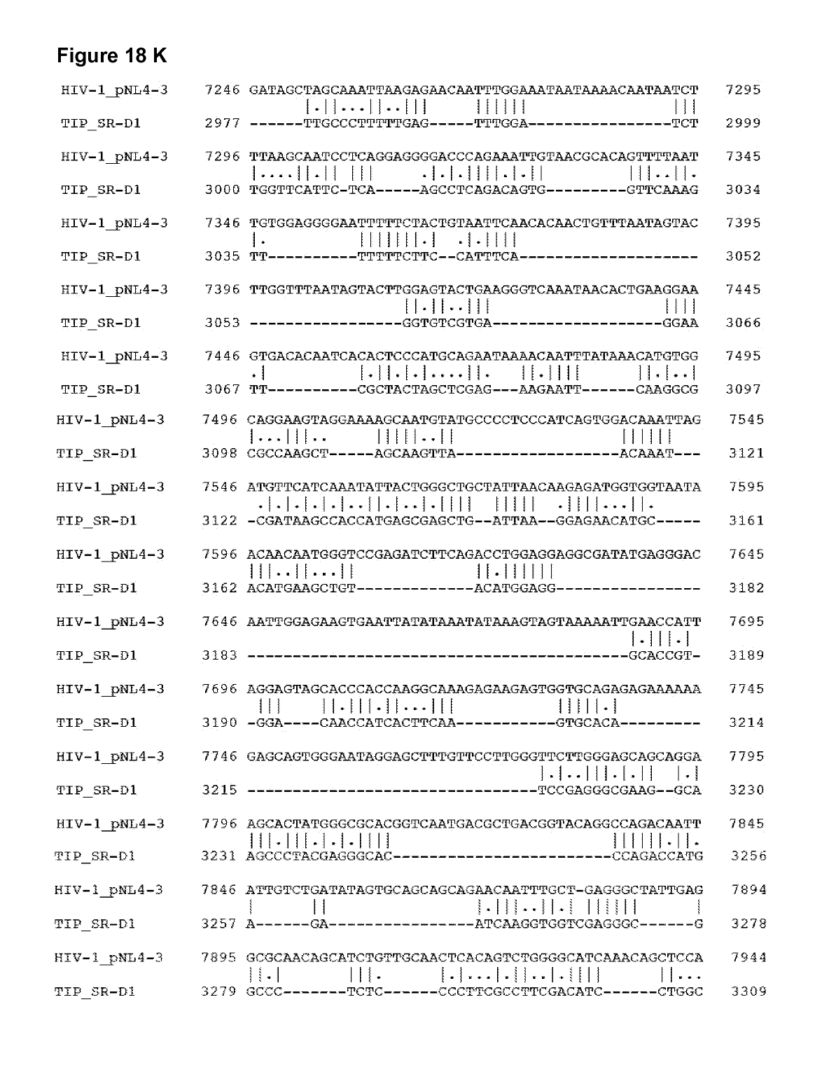

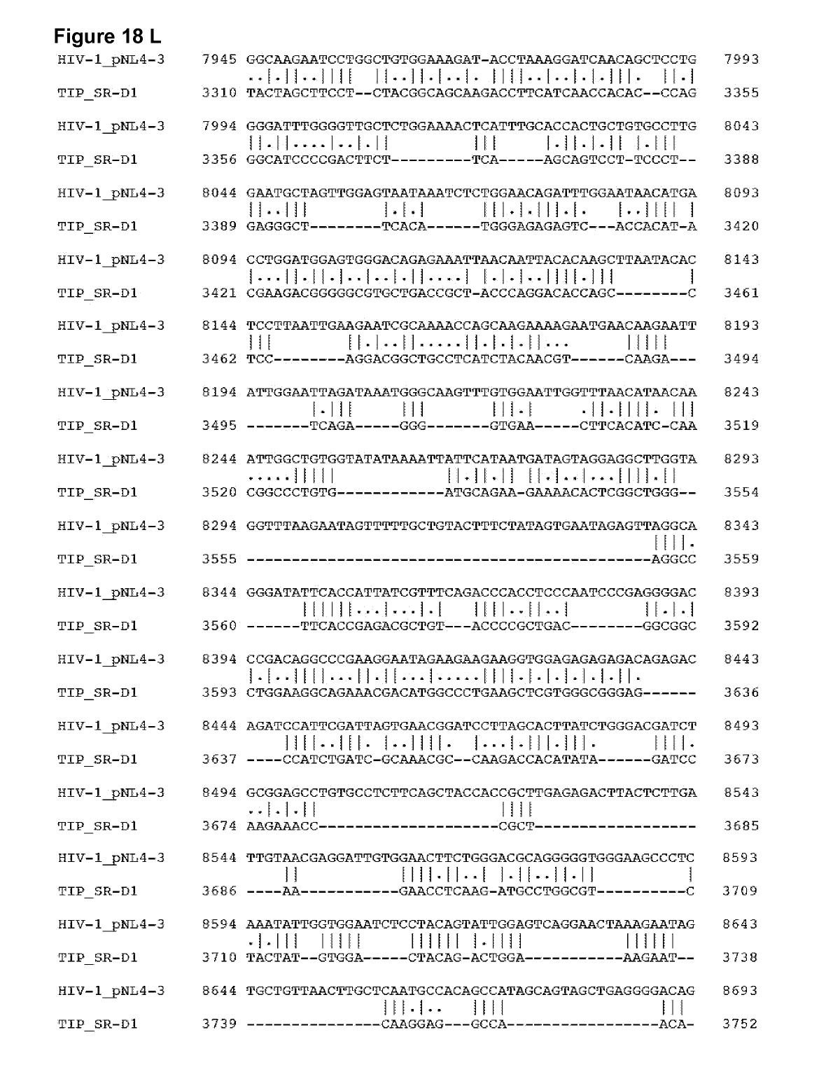

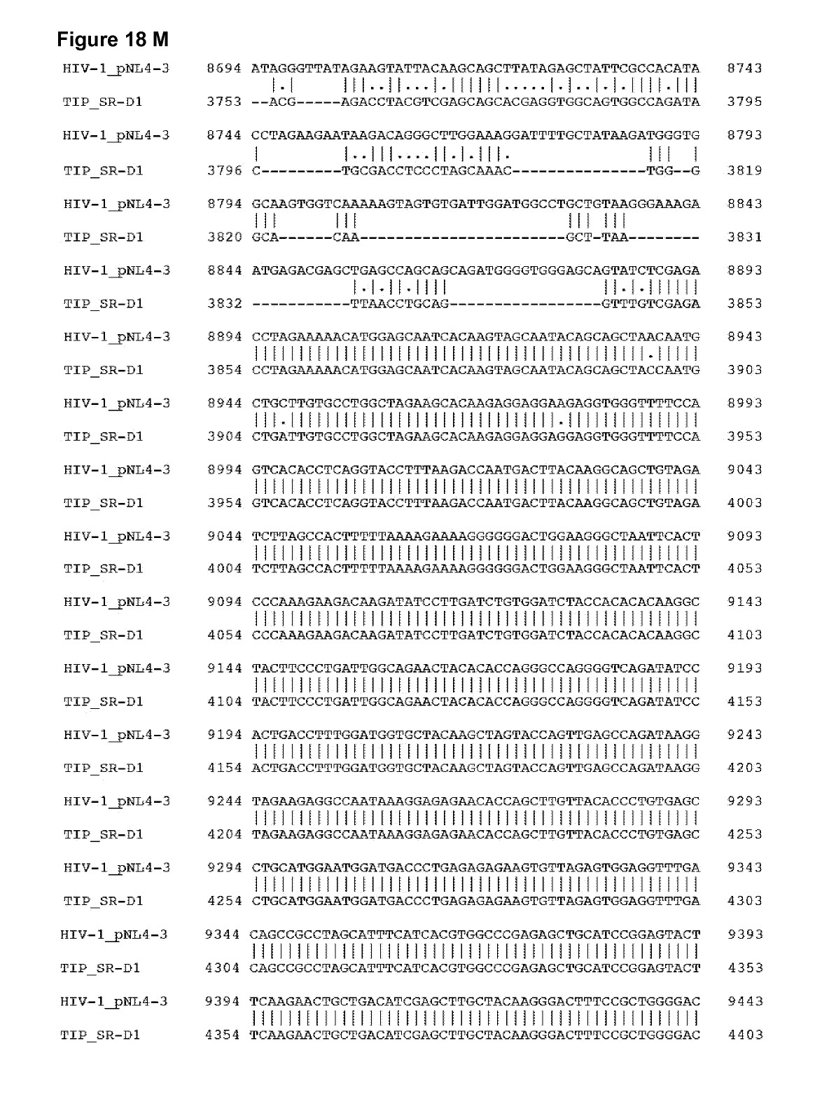

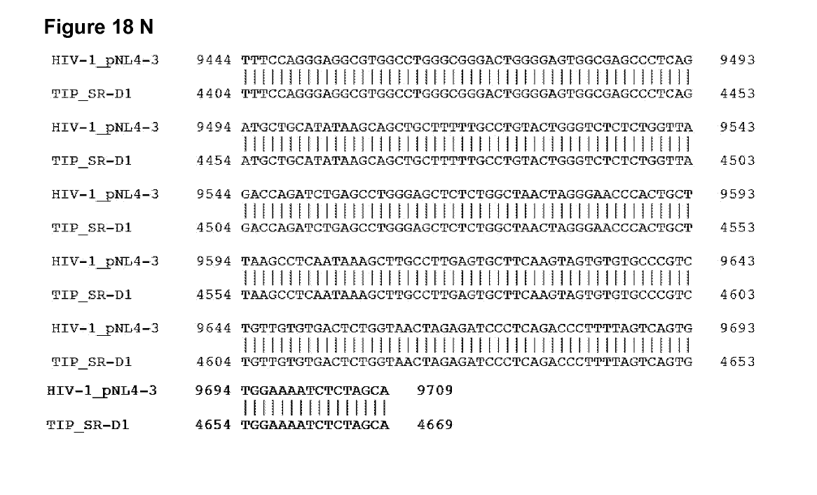

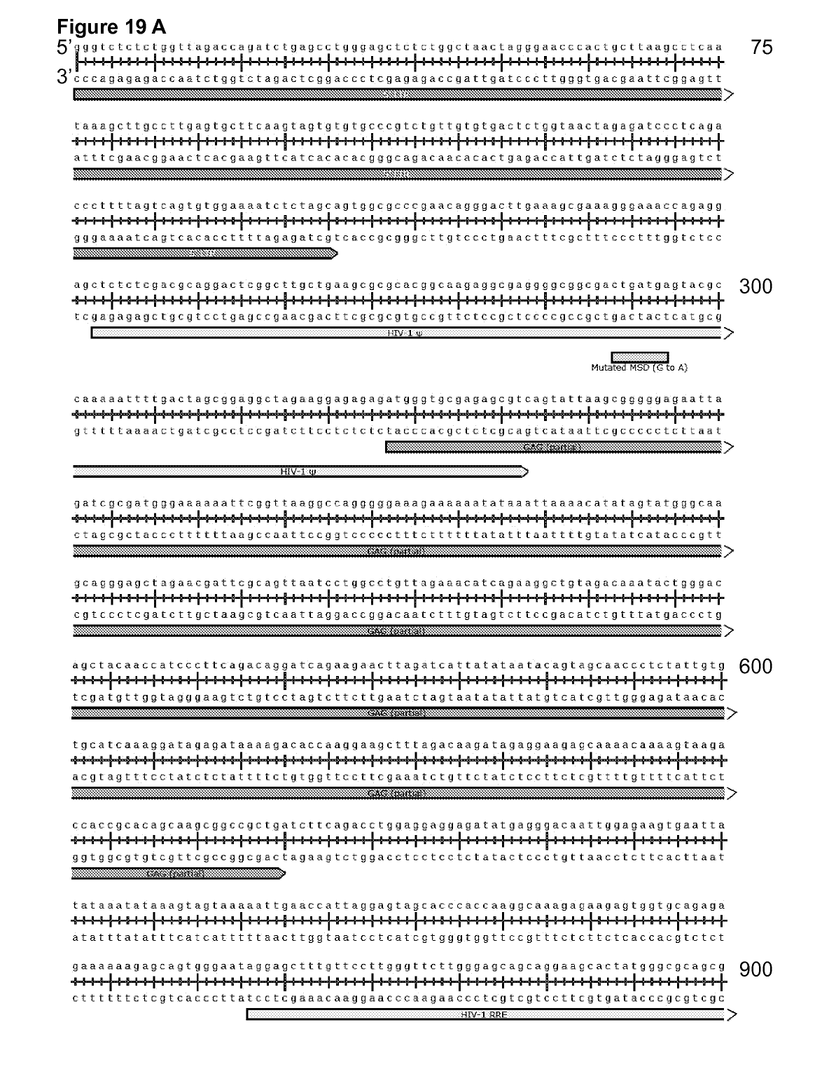

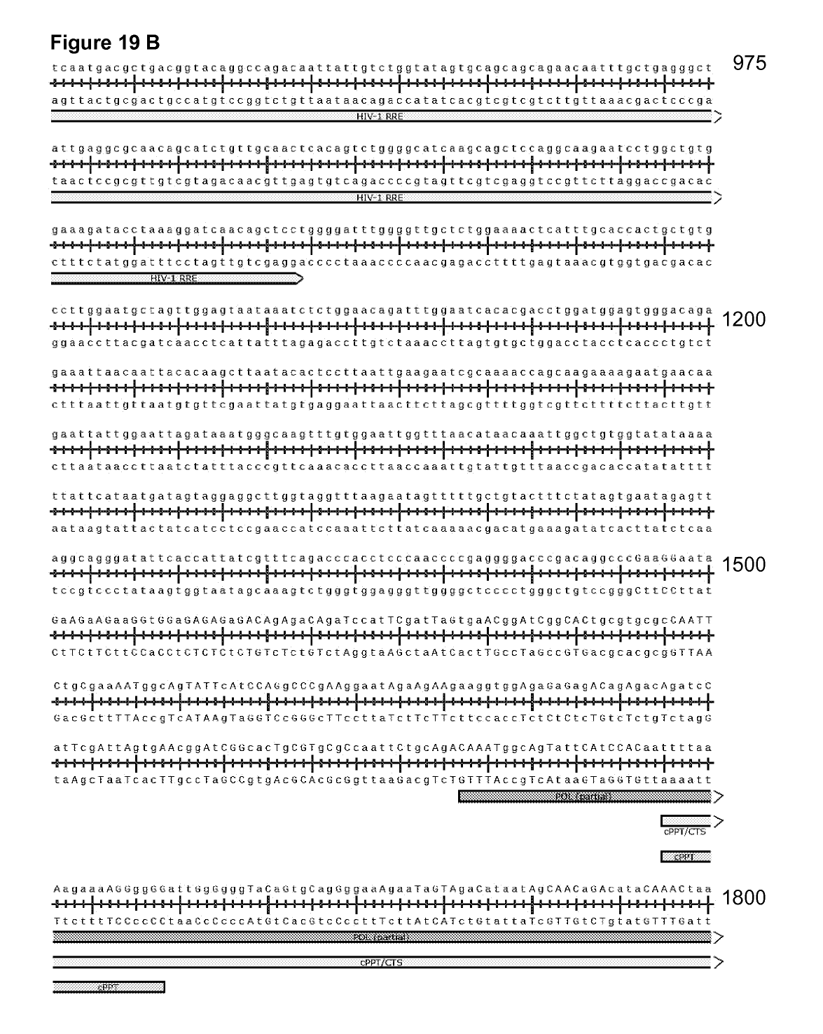

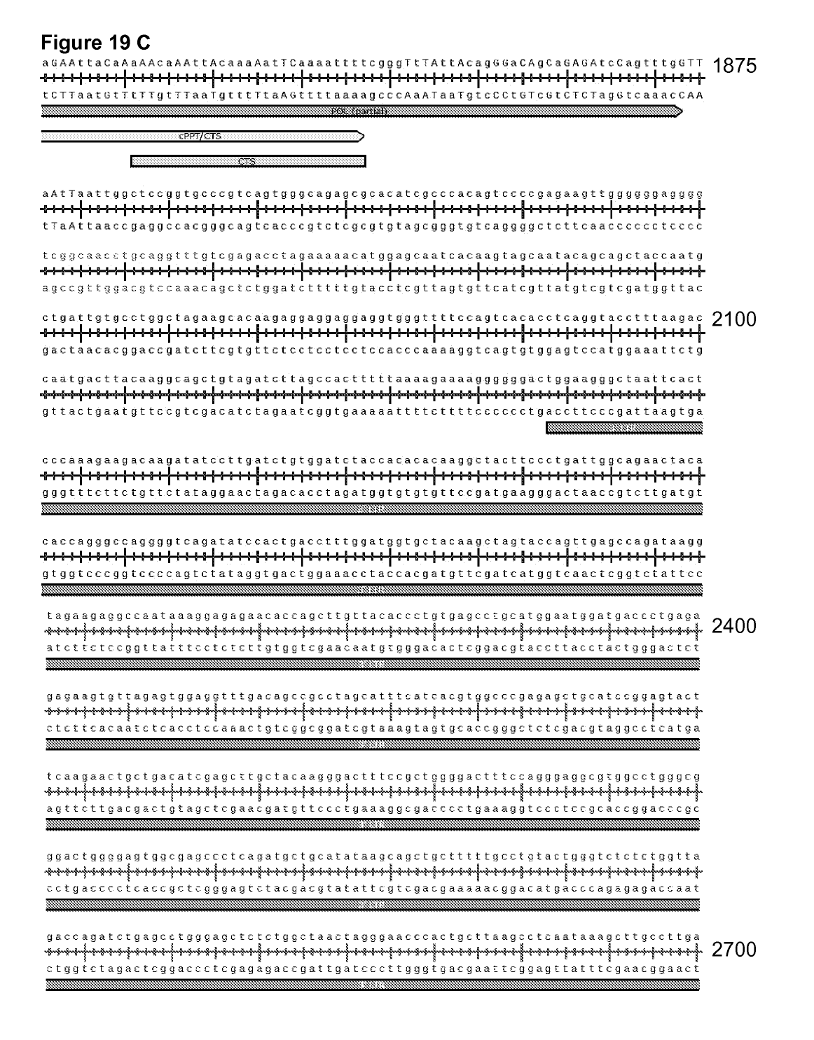

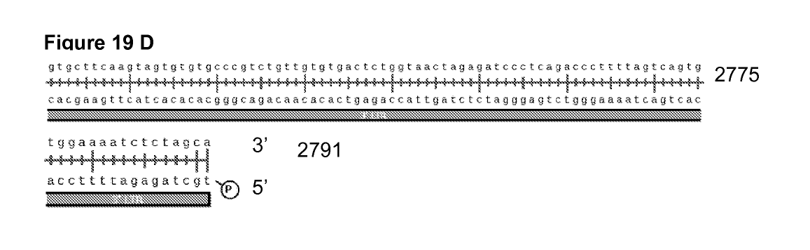

Exemplary interfering constructs are depicted in FIG. 17, FIG. 18, and FIG. 19.

### Cis-Acting Elements

An interfering construct of the present disclosure comprises lentivirus cis-acting elements. Cis-acting elements include, e.g., a lentiviral Ψ (psi) packaging signal; a lentiviral rev responsive element (rre); a lentiviral long terminal repeat (LTR); and a cis element embedded within an HIV protein-coding sequence. Nucleotide sequences for HIV-1 cis-acting elements are known in the art. See, e.g., the following web site: http://www(dot)hiv(dot)lanl(dot)gov.

Lentiviral Ψ packaging signal sequences are known in the art. See, e.g., Lever et al. (1989) J. Viro. 63:4085; and McBride et al. (1998) J. Virol. 71:4544. The Ψ packaging signal has a length of from about 80 nt to about 150 nt; and includes four stem-loop (SL) structures: SL1-SL4. A lentiviral Ψ (psi) packaging signal sequence can have at least about 85%, at least 90%, at least 95%, at least 98%, at least 99%, or 100%, nucleotide sequence identity with any known wild-type Ψ packaging signal sequence.

HIV-1 SL-1 (also known in the art as HIV-1_DIS) can have the sequence:

(SEQ ID NO: 3)
5′-GGACUCGGCUUGCUGAAGYGCRCWCRGCAAGAGGCGAGRG-3′.
HIV-1 SL2 (also known in the art as HIV-1_SD)
can have the sequence:
(SEQ ID NO: 4)
5′-RGCGACUGGUGAGUACGCH-3′.
HIV-1 SL3 can have the sequence:
(SEQ ID NO: 5)
5′-UGACUAGCGGAGGCUAGAAGGAG-3′.
HIV-1 SL4 can have the sequence:
(SEQ ID NO: 6)
5′-UGGGUGCGAGAGCGUCARUA-3′.

In the above-noted sequences, Y is C or T; W is A or T; R is A or G; and H is A, C, or T.

A lentiviral rev responsive element (rre) lies within about nt 7709-8063 of the HIV-1 genome; and has a length of from about 240 nt to about 355 nt. See, e.g., Cullen et al. (1991) J. Virol. 65: 1053; Cullen et al. (1991) Cell 58: 423-426; and Malim et al. (1989) Nature 338(6212):254-7. A suitable lentiviral rre can comprise a nucleotide sequence having at least about 85%, at least 90%, at least 95%, at least 98%, at least 99%, or 100%, nucleotide sequence identity with any known wild-type HIV-1 rre sequence.

HIV-1 LTR sequences are known in the art. A suitable lentiviral 5′-LTR or 3′-LTR can comprise a nucleotide sequence having at least about 85%, at least 90%, at least 95%, at least 98%, at least 99%, or 100%, nucleotide sequence identity with any known wild-type HIV-1 5′LTR or 3′LTR sequence.

### Dimerization Initiation Signal

Lentiviruses are diploid and genomic RNAs (gRNAs) are packaged into virions in pairs, where encapsidation of two copies of RNA is achieved by allowing the gRNAs to dimerize. This gRNA pairing is initiated at a six-nucleotide palindrome termed the dimerization initiation signal (DIS) which is located within stem loop 1 (SL1) of the HIV-1 genome and has the consensus sequence GCGCGC.

An interfering construct of the present disclosure can comprise a wildtype DIS, e.g., having the consensus sequence GCGCGC. In other cases, an interfering construct of the present disclosure comprises a DIS with a single nucleotide mutation (e.g., GCGCGC→GCGAGC). The GCGAGC DIS results in reduced HIV-1 homodimerization.

### Trans-Acting Elements

An interfering construct of the present disclosure comprises an alteration in an HIV nucleotide sequence, where the alteration renders an HIV-encoded protein selected from Env, Gag, Pol, Tat, Rev, Vpr, Nef, Vif, and Vpu non-functional.

A wild-type HIV-1 genome gives rise to three classes of RNA: unspliced RNA; incompletely spliced RNA; and fully spliced RNA.

Unspliced RNA: The unspliced 9-kb primary transcript can be expressed to generate the Gag and Gag-Pol precursor proteins or be packaged into virions to serve as the genomic RNA.

Incompletely spliced RNA. These mRNAs use the splice donor site located nearest the 5′ end of the HIV RNA genome in combination with any of the splice acceptors located in the central region of the virus. These RNAs can potentially express Env, Vif, Vpu, Vpr, and the single-exon form of Tat. These heterogeneous mRNAs are 4- to 5-kb long and retain the second intron of HIV.

Fully spliced RNA. These mRNAs have spliced out both introns of HIV and have the potential to express Rev, Nef, and the two-exon form of Tat. These heterogeneous mRNAs do not require the expression of the Rev protein.

In some cases, the alteration in the HIV nucleotide sequence is a deletion of one or more nucleotides in a splice donor and/or a splice acceptor. See, e.g., Schwartz et al. (1990) J. Virol. 64:2519. For example, in some instances, the alteration is a deletion or a substitution of one or more nucleotides in the 5′ major splice donor. Nucleotide sequences of the 5′ major splice donor of HIV are known. See, e.g., Harrison and Lever (1992) J. Virol. 66:4144.

An interfering construct of the present disclosure can in some embodiments include a deletion of one or more nucleotides in one or more splice donor and/or splice acceptor sequences of HIV, such that one or more of Env, Gag, Pol, Tat, Rev, Vpr, Nef, Vif, and Vpu are not produced. In some instances, none of Env, Gag, Pol, Tat, Rev, Vpr, Nef, Vif, and Vpu is produced. In some instances, Env, Vif, Vpu, Vpr, and Tat are not produced.

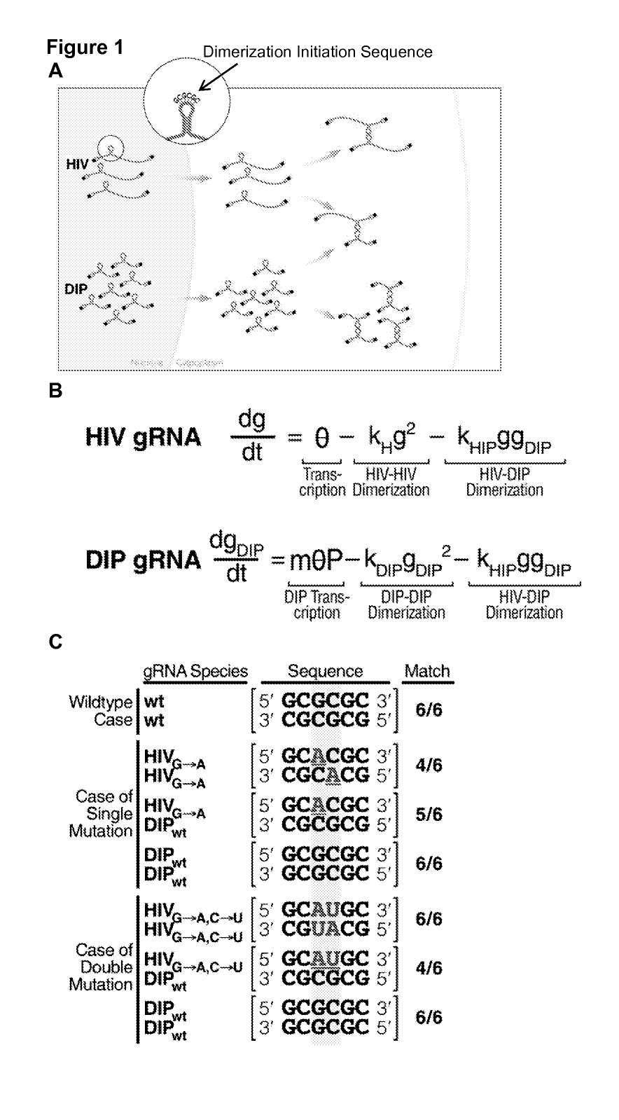

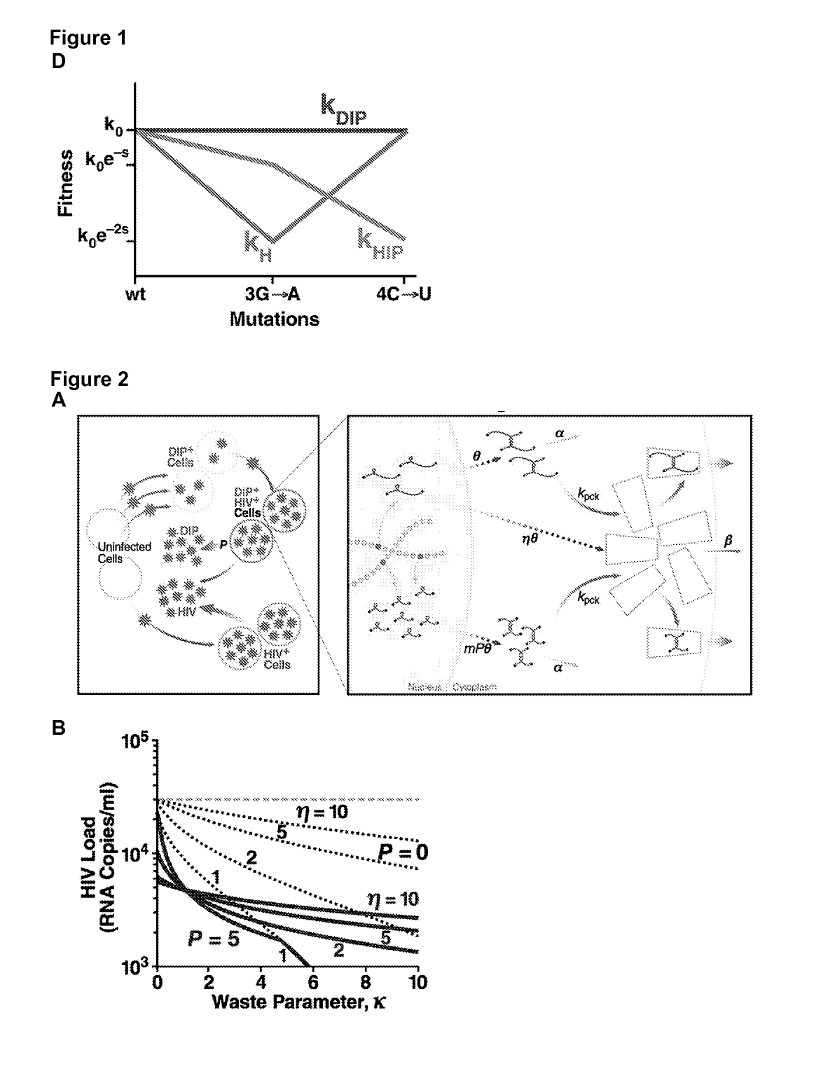

In some instances, an interfering construct of the present disclosure includes a deletion or a substitution of one or more nucleotides in an HIV splice donor selected from D1, D2, D3, and D4. In some instances, an interfering construct of the present disclosure includes a deletion or a substitution of one or more nucleotides in an HIV splice acceptor selected from A1, A2, A3, A4, A5, A6, and A7. See, e.g., FIG. 1 of Mandal et al. ((2010) J. Virol. 84:12790) for the organization of HIV splice donors D1-D4 and splice acceptors A1-A7, relative to locations of exons, in the HIV genome.

In some instances, an interfering construct of the present disclosure includes a deletion or a substitution of one or more nucleotides in the HIV major splice donor, where exemplary wild-type sequences surrounding the major splice donor (D1) include:

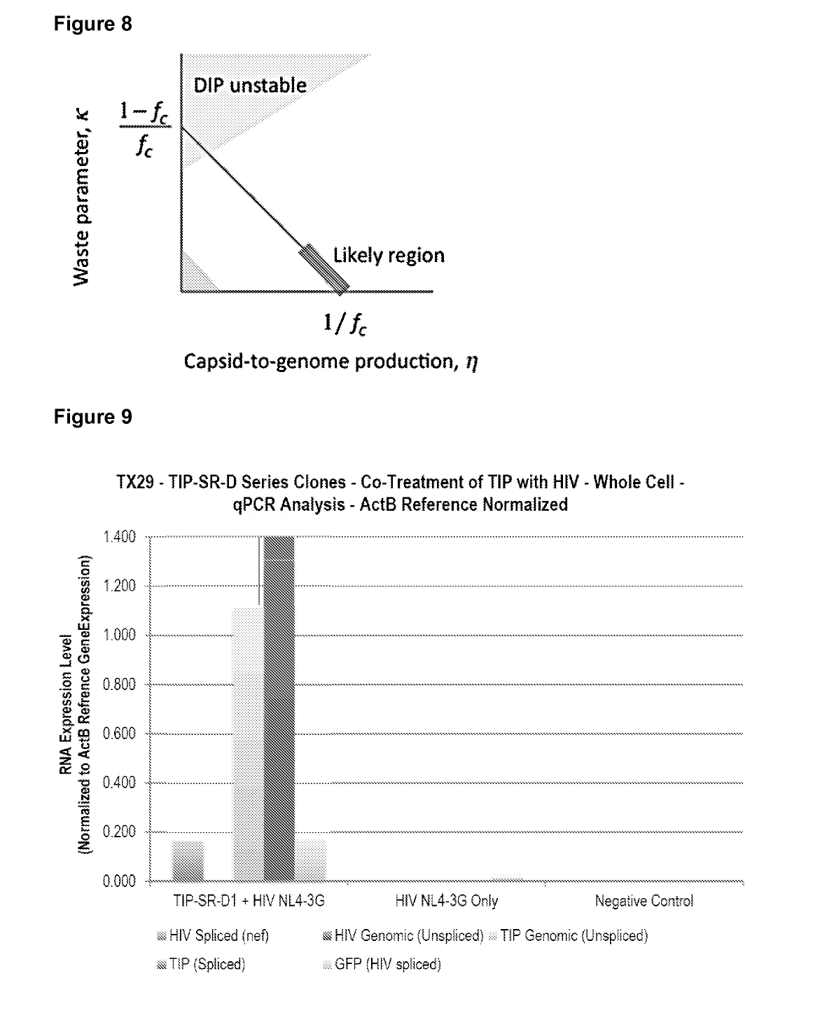

(SEQ ID NO: 7)
5′-ggcgactgGtgagtacgcc-3′,;
(SEQ ID NO: 8)
5′-ggcggctgGtgagtacgcc-3′,;
(SEQ ID NO: 9)
5′-ggcgaatgGtgagtacgcc-3′ (HIVRF),
(SEQ ID NO: 10)
5′-agcgactgGtgagtacgct-3′,;
and
(SEQ ID NO: 11)
5′-agcgaccgGtgagtacgct-3′ (HIV2226),;,

where the “G” in upper case and bold is the major splice donor. See, e.g., FIG. 8 of Harrison and Lever (1992) J. Virol. 66:4144. The major splice donor is approximately 50 nucleotides (e.g., 45 nucleotides to 55 nucleotides) 5′ of the gag initiator ATG.

In some instances, an interfering construct of the present disclosure includes a deletion or a substitution of one or more nucleotides in an HIV splice donor D2. An exemplary nucleotide sequence surrounding HIV splice donor D2 is 5′-AAGGUGAAGGG-3′ (SEQ ID NO:12). In some instances, an interfering construct of the present disclosure includes a deletion or a substitution of one or more nucleotides in an HIV splice donor D3. An exemplary nucleotide sequence surrounding HIV splice donor D3 is 5′-AAGGUAGGUCA-3′ (SEQ ID NO:13). In some instances, an interfering construct of the present disclosure includes a deletion or a substitution of one or more nucleotides in an HIV splice donor D4. An exemplary nucleotide sequence surrounding HIV splice donor D4 is 5′-CUAGACUAGAG-3′ (SEQ ID NO:14). See, e.g., Mandal et al. (2010) J. Virol. 84:12790. Another exemplary nucleotide sequence surrounding HIV splice donor D4 is 5′-GCAGUAAGUAG-3′ (SEQ ID NO:15); see, e.g., Kammler et al. (2006) Retrovirol. 3:89.

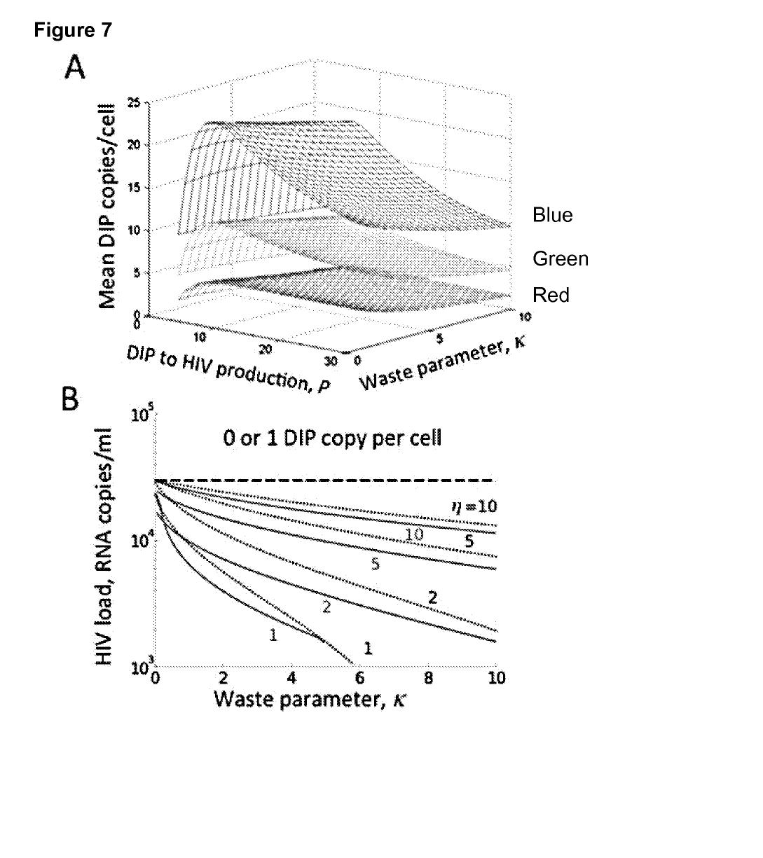

In some instances, an interfering construct of the present disclosure includes a deletion or a substitution of one or more nucleotides in an HIV splice acceptor sequence. Nucleotide sequences surrounding HIV splice acceptor sequences are known in the art. See, e.g., FIG. 7 of Schwartz et al. (1990) J. Virol. 64:2519.

For example, in the sequence: 5′-ataacaaaAGccttAGgcatctcctatggcAGgaagaagagagttAGgcAGggatattcaccattatcgtttcAGcc-3′ (SEQ ID NO:16), the “AG” in upper case and bold indicate, from 5′-3′, splice acceptors 4A, 4B, 5, 7A, 7B, and 7. The rev initiator ATG is underlined.

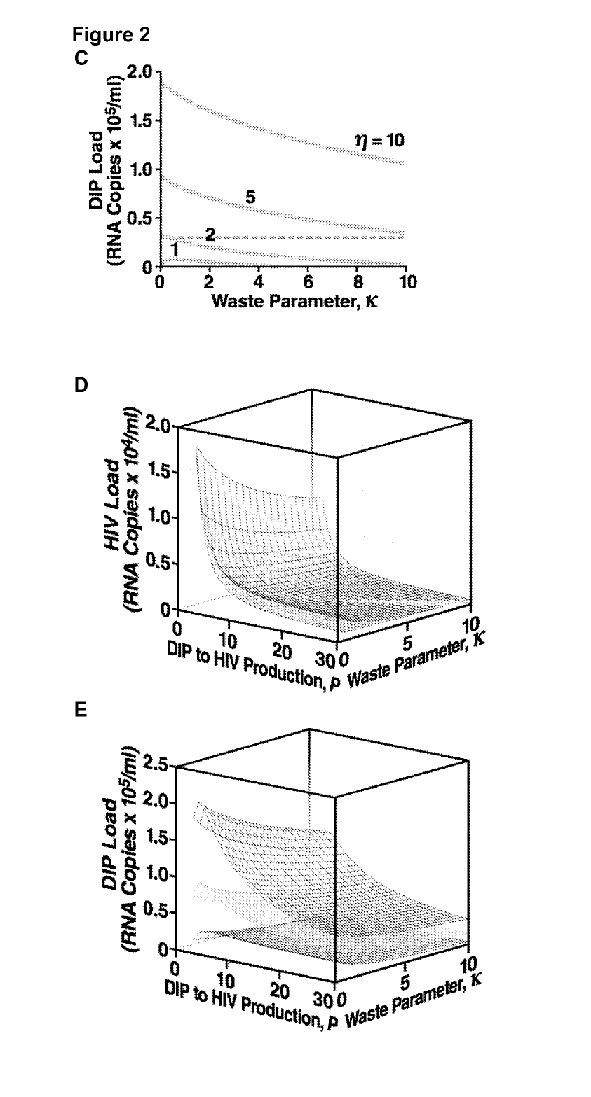

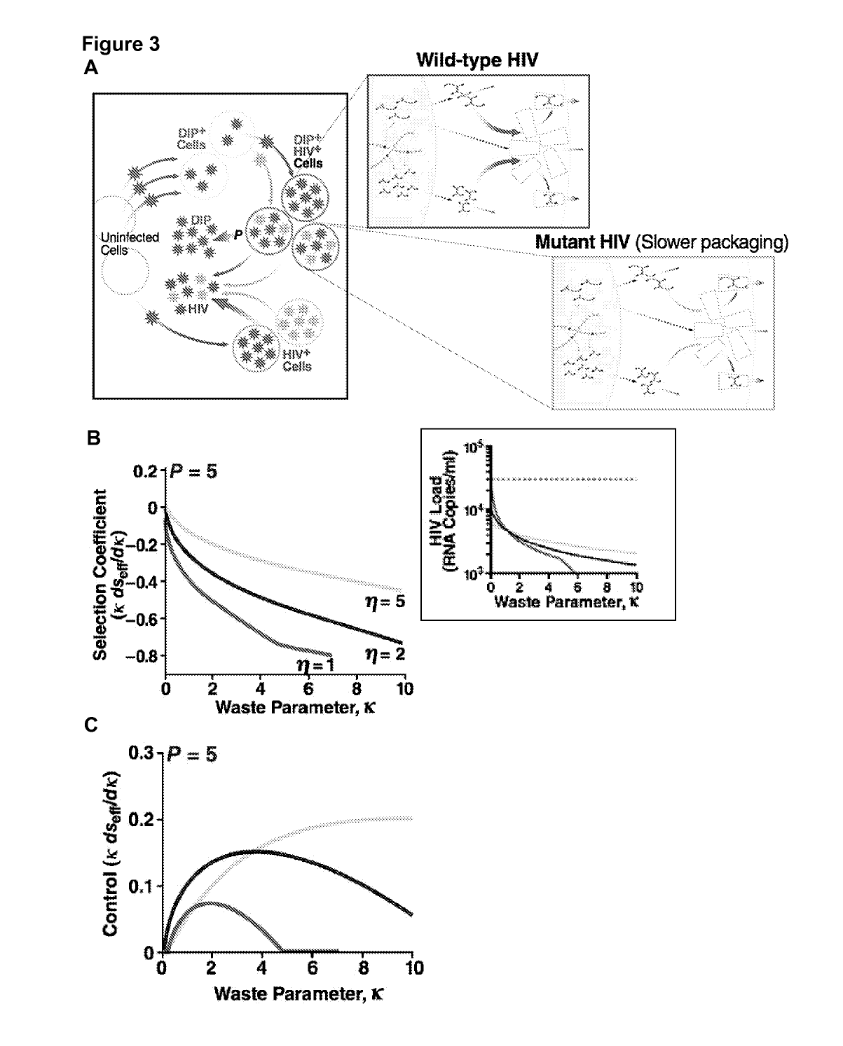

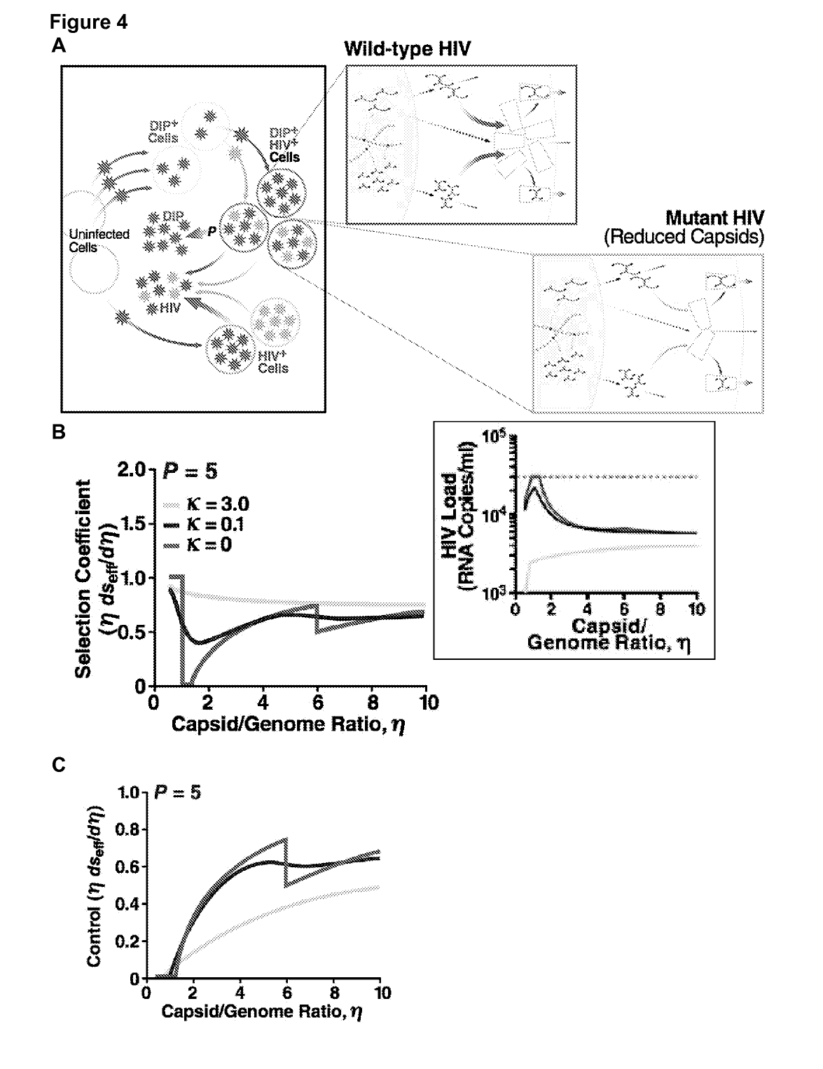

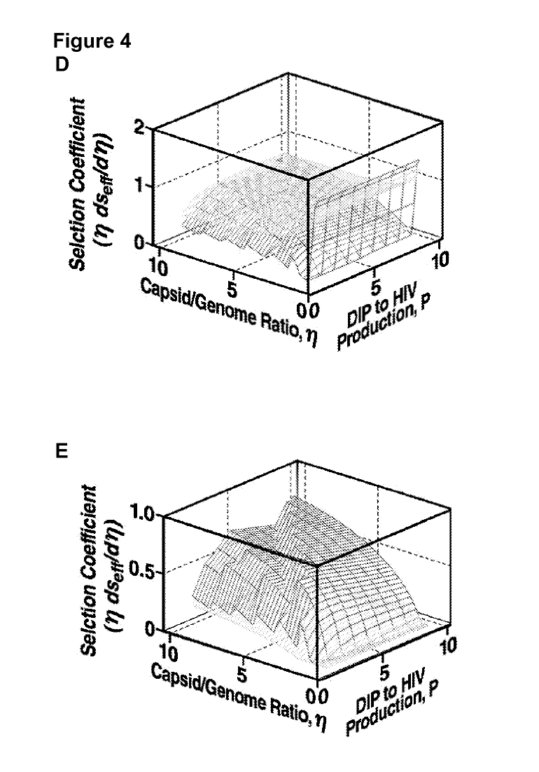

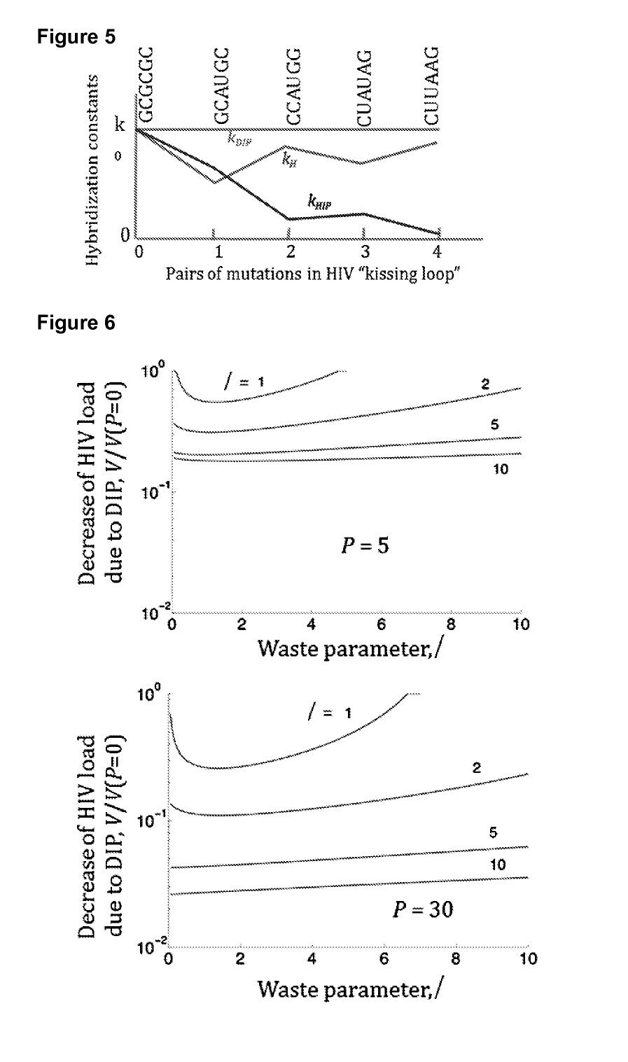

An exemplary nucleotide sequence surrounding HIV splice acceptor A7 is shown in FIG. 2A of Kammler et al. (2006) Retrovirol. 3:89. An exemplary nucleotide sequence surrounding HIV splice acceptor A5 is shown in FIG. 4A of Kammler et al. (2006) Retrovirol. 3:89. Exemplary nucleotide sequences surrounding HIV splice acceptors A1-A7 are shown in FIG. 5A of Kammler et al. (2006) Retrovirol. 3:89.

### Exemplary Interfering Constructs

Non-limiting examples of interfering constructs are depicted schematically in FIG. 17. Exemplary interfering constructs are designated “TIP (SR2)” (SEQ ID NO: 20); “TIP (SR2-D1)” (SEQ ID NO: 2) (also referred to herein as “TIP (SR-D1)” and “TIP (SR-D)”); “TIP (SR2-D1-delEF1a)” (SEQ ID NO: 18); and “TIP (SR2-D1-delEF1a-delmTagBFP2)” (SEQ ID NO: 19). Exemplary interfering constructs and sequences are depicted in FIG. 17, FIG. 18, and FIG. 19.

In some cases, an interfering construct of the present disclosure comprises a nucleotide sequence having at least 85%, at least 90%, at least 95%, at least 98%, at least 99%, or 100%, nucleotide sequence identity to the nucleotide sequence set forth in SEQ ID NO:2. In some cases, an interfering construct of the present disclosure comprises a nucleotide sequence having at least 85%, at least 90%, at least 95%, at least 98%, at least 99%, or 100%, nucleotide sequence identity to the nucleotide sequence set forth in SEQ ID NO:18. In some cases, an interfering construct of the present disclosure comprises a nucleotide sequence having at least 85%, at least 90%, at least 95%, at least 98%, at least 99%, or 100%, nucleotide sequence identity to the nucleotide sequence set forth in SEQ ID NO:19.

Interfering Particles

The present disclosure further comprises a particle comprising an interfering construct. Such a particle is referred to herein as an “interfering particle.” An interfering particle is capable of infecting and entering a host cell.

An interfering particle of the present disclosure will in some cases comprise HIV envelope proteins (e.g., gp120 and gp41). In other cases, an interfering particle of the present disclosure will comprise a non-HIV envelope protein, i.e., the interfering construct will be pseudotyped.

In some cases, an interfering particle of the present disclosure will comprise HIV envelope proteins (e.g., gp120 and gp41), such that the packaged interfering particle will infect cells via the same receptor (CCR5) used by wild-type HIV. CCR5 is predominantly expressed on T cells, macrophages, dendritic cells and microglia. Thus, in some embodiments, a subject interfering particle will infect primarily T cells, macrophages, dendritic cells and microglia.

Pseudotyped Interfering Constructs

As noted above, in some instances, a subject interfering construct will be pseudotyped.

For example, in some instances, a subject interfering construct is pseudotyped with VSV-G. VSV-G pseudotyped retroviruses demonstrate a broad host range (pantropic) and are able to efficiently infect cells that are resistant to infection by ecotropic and amphotropic retroviruses. (Yee et al. (2004) Proc. Natl. Acad. Sci. USA 91:9564-9568. Any suitable serotype (e.g., Indiana, New Jersey, Chandipura, Piry) and strain (e.g., VSV Indiana, San Juan) of VSV-G can be used. Stable VSV-G pseudotyped retrovirus packaging cell lines permit generation of large scale viral preparations (e.g. from 10 to 50 liters supernatant) to yield retroviral stocks in the range of 10⁷ to 10¹¹ retroviral particles per ml.

In some embodiments, an interfering construct of the present disclosure is pseudotyped with a Sindbis virus envelope glycoprotein. See, e.g., U.S. Pat. No. 8,187,872.

### Cell Lines

Any suitable cell line can be employed to prepare packaging cells for use in packaging a subject interfering construct to generate an interfering construct-containing infectious particle (an “interfering particle”). Generally, the cells are mammalian cells. In a particular embodiment, the cells used to produce the packaging cell line are human cells. Suitable human cell lines which can be used include, for example, 293 cells (Graham et al. (1977) J. Gen. Virol., 36:59-72, tsa 201 cells (Heinzel et al. (1988) J. Virol., 62:3738), and NIH3T3 cells (ATCC)). Other suitable packaging cell lines for use in the present invention include other human cell line derived (e.g., embryonic cell line derived) packaging cell lines and murine cell line derived packaging cell lines, such as Psi-2 cells (Mann et al. (1983) Cell, 33:153-159; FLY (Cossett et al. (1993) Virol., 193:385-395; BOSC 23 cells (Pear et al. (1993) PNAS 90:8392-8396; PA317 cells (Miller et al. (1986) Molec. and Cell. Biol., 6:2895-2902; Kat cell line (Finer et al. (1994) Blood, 83:43-50; GP+E cells and GP+EM12 cells (Markowitz et al. (1988) J. Virol., 62:1120-1124, and Psi Crip and Psi Cre cells (U.S. Pat. No. 5,449,614; Danos, O. and Mulligan et al. (1988) PNAS 85:6460-6464). Packaging cell lines can produce retroviral particles having a pantropic, amphotropic, or ecotropic host range. Exemplary packaging cell lines produce retroviral particles, such as lentiviral particles (e.g., HIV-1, HIV-2 and SIV) capable of infecting dividing, as well as non-dividing cells.

### Compositions

The present disclosure provides compositions, including pharmaceutical compositions and biological compositions, comprising a subject interfering construct or a subject interfering particle. For simplicity, a subject interfering construct and a subject interfering particle are referred to collectively below as “active agent.”

The present disclosure provides a composition comprising a subject interfering construct or a subject interfering particle. A subject interfering construct composition or a subject interfering particle composition can comprise, in addition to a subject interfering construct or a subject interfering particle, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; glycerol; and the like.

### Pharmaceutical Compositions

An active agent is in some embodiments formulated with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. For the purposes of the following description of formulations, “active agent” includes an active agent as described above, and optionally one or more additional therapeutic agent.

In a subject method, an active agent may be administered to the host using any convenient means capable of resulting in the desired degree of reduction of immunodeficiency virus transcription. Thus, an active agent can be incorporated into a variety of formulations for therapeutic administration. For example, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In an exemplary embodiment, an active agent is formulated as a gel, as a solution, or in some other form suitable for intravaginal administration. In a further exemplary embodiment, an active agent is formulated as a gel, as a solution, or in some other form suitable for rectal (e.g., intrarectal) administration.

In pharmaceutical dosage forms, an active agent may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, an active is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the active agent(s) in a composition as a solution in sterile water, normal saline or another pharmaceutically acceptable carrier.

Unit dosage forms for intravaginal or intrarectal administration such as syrups, elixirs, gels, and suspensions may be provided wherein each dosage unit, for example, teaspoonful, tablespoonful, tablet, unit gel volume, or suppository, contains a predetermined amount of the composition containing one or more active agents.

The term “unit dosage form,” as used herein, refers to physically discrete units suitable as unitary dosages for human and animal subjects, each unit containing a predetermined quantity of an active agent, calculated in an amount sufficient to produce the desired effect in association with a pharmaceutically acceptable diluent, carrier or vehicle. The specifications for a given active agent will depend in part on the particular compound employed and the effect to be achieved, and the pharmacodynamics associated with each compound in the host.

Other modes of administration will also find use with the subject invention. For instance, an active agent can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g. about 1% to about 2%.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

An active agent will in some embodiments be formulated for vaginal delivery. A subject formulation for intravaginal administration comprises an active agent formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

An active agent will in some embodiments be formulated for rectal delivery. A subject formulation for intrarectal administration comprises an active agent formulated as an intrarectal bioadhesive tablet, intrarectal bioadhesive microparticle, intrarectal cream, intrarectal lotion, intrarectal foam, intrarectal ointment, intrarectal paste, intrarectal solution, or intrarectal gel.

A subject formulation comprising an active agent includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Biological Compositions

The present disclosure provides a biological composition comprising: a) a subject interfering construct or a subject interfering particle; and b) a biological fluid.

Suitable biological fluids include, e.g., blood or a blood fraction. Blood fractions include, e.g., serum and plasma. In some cases, the biological fluid has been isolated from an individual. In some cases, the biological fluid has been subjected to one or more processing steps, e.g., removal of pathogen(s) such as HCV, HIV, and the like.

### Treatment and Prophylactic Methods

The present disclosure provides a method of reducing human immunodeficiency virus viral load in an individual. The method generally involves administering to the individual an effective amount of a subject interfering construct, a pharmaceutical formulation comprising a subject interfering construct, a subject interfering particle, or a pharmaceutical formulation comprising a subject interfering particle.

In some cases, a subject method involves administering to an individual in need thereof an effective amount of a subject interfering particle, or a pharmaceutical formulation comprising a subject interfering particle. In some cases, an effective amount of a subject interfering particle is an amount that, when administered to an individual in one or more doses, in monotherapy or in combination therapy, is effective to reduce immunodeficiency virus load in the individual by at least about 10%, at least about 20%, at least about 25%, at least about 30%, at least about 40%, at least about 50%, at least about 60%, at least about 70%, at least about 80%, or greater than 80%, compared to the immunodeficiency virus load in the individual in the absence of treatment with the interfering particle.

In some cases, a subject method involves administering to an individual in need thereof an effective amount of a subject interfering particle. In some embodiments, an “effective amount” of a subject interfering particle is an amount that, when administered to an individual in one or more doses, in monotherapy or in combination therapy, is effective to increase the number of CD4⁺ T cells in the individual by at least about 20%, at least about 25%, at least about 30%, at least about 40%, at least about 50%, at least about 60%, at least about 70%, at least about 80%, at least about 2-fold, at least about 2.5-fold, at least about 3-fold, at least about 5-fold, at least about 10-fold, or greater than 10-fold, compared to the number of CD4⁺ T cells in the individual in the absence of treatment with the interfering particle.

Any of a variety of methods can be used to determine whether a treatment method is effective. For example, methods of determining whether the methods of the invention are effective in reducing immunodeficiency virus (e.g., HIV) viral load, and/or treating an immunodeficiency virus (e.g., HIV) infection, are any known test for indicia of immunodeficiency virus (e.g., HIV) infection, including, but not limited to, measuring viral load, e.g., by measuring the amount of immunodeficiency virus (e.g., HIV) in a biological sample, e.g., using a polymerase chain reaction (PCR) with primers specific for an immunodeficiency virus (e.g., HIV) polynucleotide sequence; detecting and/or measuring a polypeptide encoded by an immunodeficiency virus (e.g., HIV), e.g., p24, gp120, reverse transcriptase, using, e.g., an immunological assay such as an enzyme-linked immunosorbent assay (ELISA) with an antibody specific for the polypeptide; and measuring the CD4⁺ T cell count in the individual.

### Formulations, Dosages, and Routes of Administration

Prior to introduction into a host, an interfering construct or an interfering particle can be formulated into various compositions for use in therapeutic and prophylactic treatment methods. In particular, the interfering construct or interfering particle can be made into a pharmaceutical composition by combination with appropriate pharmaceutically acceptable carriers or diluents, and can be formulated to be appropriate for either human or veterinary applications. For simplicity, a subject interfering construct and a subject interfering particle are collectively referred to below as “active agent” or “active ingredient.”

Thus, a composition for use in a subject treatment method can comprise a subject interfering construct or subject interfering particle, e.g., in combination with a pharmaceutically acceptable carrier. Pharmaceutically acceptable carriers are well known to those skilled in the art, as are suitable methods of administration. The choice of carrier will be determined, in part, by the particular vector, as well as by the particular method used to administer the composition. One skilled in the art will also appreciate that various routes of administering a composition are available, and, although more than one route can be used for administration, a particular route can provide a more immediate and more effective reaction than another route. Accordingly, there are a wide variety of suitable formulations of a subject interfering construct composition or a subject interfering particle composition.

A composition a subject interfering construct or subject interfering particle, alone or in combination with other antiviral compounds, can be made into a formulation suitable for parenteral administration. Such a formulation can include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be provided in unit dose or multidose sealed containers, such as ampules and vials, and can be stored in a freeze-dried (lyophilized) condition requiring only the addition of the sterile liquid carrier, for example, water, for injections, immediately prior to use. Injectable solutions and suspensions can be prepared from sterile powders, granules, and tablets, as described herein.

A formulation suitable for oral administration can be a liquid solution, such as an effective amount of a subject interfering construct or a subject interfering particle dissolved in diluents, such as water, saline, or fruit juice; capsules, sachets or tablets, each containing a predetermined amount of the active agent (a subject interfering construct or subject interfering particle), as solid or granules; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

An aerosol formulation suitable for administration via inhalation also can be made. The aerosol formulation can be placed into a pressurized acceptable propellant, such as dichlorodifluoromethane, propane, nitrogen, and the like.

Similarly, a formulation suitable for oral administration can include lozenge forms, that can comprise the active ingredient in a flavor, usually sucrose and acacia or tragacanth; pastilles comprising the active ingredient (a subject interfering construct or subject interfering particle) in an inert base, such as gelatin and glycerin, or sucrose and acacia; and mouthwashes comprising the active agent in a suitable liquid carrier; as well as creams, emulsions, gels, and the like containing, in addition to the active agent, such carriers as are known in the art.

A formulation suitable for topical application can be in the form of creams, ointments, or lotions.

A formulation for rectal administration can be presented as a suppository with a suitable base comprising, for example, cocoa butter or a salicylate. A formulation suitable for vaginal administration can be presented as a pessary, tampon, cream, gel, paste, foam, or spray formula containing, in addition to the active ingredient, such carriers as are known in the art to be appropriate. Similarly, the active ingredient can be combined with a lubricant as a coating on a condom.

The dose administered to an animal, particularly a human, in the context of the present invention should be sufficient to effect a therapeutic response in the infected individual over a reasonable time frame. The dose will be determined by the potency of the particular interfering construct or interfering particle employed for treatment, the severity of the disease state, as well as the body weight and age of the infected individual. The size of the dose also will be determined by the existence of any adverse side effects that can accompany the use of the particular interfering construct or interfering particle employed. It is always desirable, whenever possible, to keep adverse side effects to a minimum.

The dosage can be in unit dosage form, such as a tablet, a capsule, a unit volume of a liquid formulation, etc. The term “unit dosage form” as used herein refers to physically discrete units suitable as unitary dosages for human and animal subjects, each unit containing a predetermined quantity of an interfering construct or an interfering particle, alone or in combination with other antiviral agents, calculated in an amount sufficient to produce the desired effect in association with a pharmaceutically acceptable diluent, carrier, or vehicle. The specifications for the unit dosage forms of the present disclosure depend on the particular construct or particle employed and the effect to be achieved, as well as the pharmacodynamics associated with each construct or particle in the host. The dose administered can be an “antiviral effective amount” or an amount necessary to achieve an “effective level” in the individual patient.

Generally, an amount of a subject interfering construct or a subject interfering particle sufficient to achieve a tissue concentration of the administered construct or particle of from about 50 mg/kg to about 300 mg/kg of body weight per day can be administered, e.g., an amount of from about 100 mg/kg to about 200 mg/kg of body weight per day. In certain applications, e.g., topical, ocular or vaginal applications, multiple daily doses can be administered. Moreover, the number of doses will vary depending on the means of delivery and the particular interfering construct or interfering particle administered.

### Combination Therapy

In some embodiments, a subject interfering construct or interfering particle (or composition comprising same) is administered in combination therapy with one or more additional therapeutic agents. Suitable additional therapeutic agents include agents that inhibit one or more functions of an immunodeficiency virus; agents that treat or ameliorate a symptom of an immunodeficiency virus infection; agents that treat an infection that may occur secondary to an immunodeficiency virus infection; and the like.

Therapeutic agents include, e.g., beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddl, Videx™), stavudine (d4T, Zerit™), zalcitabine (dideoxycytosine, ddC, Hivid™) nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™) ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. Anti-HIV agents are those in the preceding list that specifically target a function of one or more HIV proteins.

In some embodiments, a subject active agent is administered in combination therapy with two or more anti-HIV agents. For example, a subject active agent can be administered in combination therapy with one, two, or three nucleoside reverse transcriptase inhibitors (e.g., Combivir, Epivir, Hivid, Retrovir, Videx, Zerit, Ziagen, etc.). A subject active agent can be administered in combination therapy with one or two non-nucleoside reverse transcriptase inhibitors (e.g., Rescriptor, Sustiva, Viramune, etc.). A subject active agent can be administered in combination therapy with one or two protease inhibitors (e.g., Agenerase, Crixivan, Fortovase, Invirase, Kaletra, Norvir, Viracept, etc.). A subject active agent can be administered in combination therapy with a protease inhibitor and a nucleoside reverse transcriptase inhibitor. A subject active agent can be administered in combination therapy with a protease inhibitor, a nucleoside reverse transcriptase inhibitor, and a non-nucleoside reverse transcriptase inhibitor. A subject active agent can be administered in combination therapy with a protease inhibitor and a non-nucleoside reverse transcriptase inhibitor. Other combinations of a subject active agent with one or more of a protease inhibitor, a nucleoside reverse transcriptase inhibitor, and a non-nucleoside reverse transcriptase inhibitor are contemplated.

In some embodiments, a subject treatment method involves administering: a) a subject active agent; and b) an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a subject treatment method involves administering: a) a subject active agent; and b) an HIV inhibitor, where suitable HIV inhibitors include, but are not limited to, one or more nucleoside/nucleotide reverse transcriptase inhibitors (NRTIs), non-nucleoside reverse transcriptase inhibitors (NNRTIs), protease inhibitors (PIs), fusion inhibitors, integrase inhibitors, chemokine receptor (e.g., CXCR4, CCR5) inhibitors, and hydroxyurea.

Nucleoside reverse transcriptase inhibitors include, but are not limited to, abacavir (ABC; ZIAGEN™), didanosine (dideoxyinosine (ddl); VIDEX™), lamivudine (3TC; EPIVIR™), stavudine (d4T; ZERIT™, ZERIT XR™), zalcitabine (dideoxycytidine (ddC); HIVID™), zidovudine (ZDV, formerly known as azidothymidine (AZT); RETROVIR™), abacavir, zidovudine, and lamivudine (TRIZIVIR™), zidovudine and lamivudine (COMBIVIR™), and emtricitabine (EMTRIVA™). Nucleotide reverse transcriptase inhibitors include tenofovir disoproxil fumarate (VIREAD™). Non-nucleoside reverse transcriptase inhibitors for HIV include, but are not limited to, nevirapine (VIRAMUNE™), delavirdine mesylate (RESCRIPTOR™), and efavirenz (SUSTIVA™).

Protease inhibitors (PIs) for treating HIV infection include amprenavir (AGENERASE™), saquinavir mesylate (FORTOVASE™, INVIRASE™.), ritonavir (NORVIR™), indinavir sulfate (CRIXIVAN™), nelfmavir mesylate (VIRACEPT™), lopinavir and ritonavir (KALETRA™), atazanavir (REYATAZ™), and fosamprenavir (LEXIVA™).

Fusion inhibitors prevent fusion between the virus and the cell from occurring, and therefore, prevent HIV infection and multiplication. Fusion inhibitors include, but are not limited to, enfuvirtide (FUZEON™), Lalezari et al., New England J. Med., 348:2175-2185 (2003); and maraviroc (SELZENTRY™, Pfizer).

An integrase inhibitor blocks the action of integrase, preventing HIV-1 genetic material from integrating into the host DNA, and thereby stopping viral replication. Integrase inhibitors include, but are not limited to, raltegravir (ISENTRESS™, Merck); and elvitegravir (GS 9137, Gilead Sciences).

Maturation inhibitors include, e.g., bevirimat (3β-(3-carboxy-3-methyl-butanoyloxy) lup-20(29)-en-28-oic acid); and Vivecon (MPC9055).

In some embodiments, a subject treatment method involves administering: a) a subject active agent; and b) one or more of: (1) an HIV protease inhibitor selected from amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, R00334649, KNI-272, DPC-681, DPC-684, GW640385X, DG17, PPL-100, DG35, and AG 1859; (2) an HIV non-nucleoside inhibitor of reverse transcriptase selected from capravirine, emivirine, delaviridine, efavirenz, nevirapine, (+) calanolide A, etravirine, GW5634, DPC-083, DPC-961, DPC-963, MW-150, and TMC-120, TMC-278 (rilpivirene), efavirenz, BILR 355 BS, VRX 840773, UK-453061, and RDEA806; (3) an HIV nucleoside inhibitor of reverse transcriptase selected from zidovudine, emtricitabine, didanosine, stavudine, zalcitabine, lamivudine, abacavir, amdoxovir, elvucitabine, alovudine, MIV-210, racivir, D-d4FC, emtricitabine, phosphazide, fozivudine tidoxil, apricitibine (AVX754), amdoxovir, KP-1461, and fosalvudine tidoxil (formerly HDP 99.0003); (4) an HIV nucleotide inhibitor of reverse transcriptase selected from tenofovir and adefovir; (5) an HIV integrase inhibitor selected from curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-538158, GSK364735C, BMS-707035, MK-2048, and BA 011; (6) a gp41 inhibitor selected from enfuvirtide, sifuvirtide, FB006M, and TRI-1144; (7) a CXCR4 inhibitor, such as AMD-070; (8) an entry inhibitor, such as SP01A; (9) a gp120 inhibitor, such as BMS-488043 and/or BlockAide/CR; (10) a G6PD and NADH-oxidase inhibitor, such as immunitin; (11) a CCR5 inhibitors selected from the group consisting of aplaviroc, vicriviroc, maraviroc, PRO-140, INCB15050, PF-232798 (Pfizer), and CCR5 mAb004; (12) another drug for treating HIV selected from BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDXO10 (ipilimumab), PBS 119, ALG 889, and PA-1050040 (PA-040); (13) any combinations or mixtures of the above.

As further examples, in some embodiments, a subject treatment method involves administering: a) a subject active agent; and b) one or more of: i) amprenavir (Agenerase; (3S)-oxolan-3-yl N-[(2S,3R)-3-hydroxy-4-[N-(2-methylpropyl)(4-aminobenzene)sulfonamido]-1-phenylbutan-2-yl]carbamate) in an amount of 600 mg or 1200 mg twice daily; ii) tipranavir (Aptivus; N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide) in an amount of 500 mg twice daily; iii) idinavir (Crixivan; (2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide) in an amount of 800 mg three times daily; iv) saquinavir (Invirase; 2S)—N-[(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide) in an amount of 1,000 mg twice daily; v) lopinavir and ritonavir (Kaleta; where lopinavir is 2S)—N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide; and ritonavir is 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl}) carbamoyl]amino}butanamido]-1,6-diphenylhexan-2-yl]carbamate) in an amount of 133 mg twice daily; vi) fosamprenavir (Lexiva; {[(2R,3S)-14N-(2-methylpropyl)(4-aminobenzene)sulfonamido]-3-({[(3S)-oxolan-3-yloxy]carbonyl}amino)-4-phenylbutan-2-yl]oxy}phosphonic acid) in an amount of 700 mg or 1400 mg twice daily); vii) ritonavir (Norvir) in an amount of 600 mg twice daily; viii) nelfinavir (Viracept; (3S,4aS,8aS)—N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide) in an amount of 750 mg three times daily or in an amount of 1250 mg twice daily; ix) Fuzeon (Acetyl-YTSLIHSLIEESQNQ QEKNEQELLELDKWASLWNWF-amide; SEQ ID NO:17) in an amount of 90 mg twice daily; x) Combivir in an amount of 150 mg lamivudine (3TC; 2′,3′-dideoxy-3′-thiacytidine) and 300 mg zidovudine (AZT; azidothymidine) twice daily; xi) emtricitabine (Emtriva; 4-amino-5-fluoro-1-[(2R,5S)-2-(hydroxymethyl)-1,3-oxathiolan-5-yl]-1,2-dihydropyrimidin-2-one) in an amount of 200 mg once daily; xii) Epzicom in an amount of 600 mg abacavir (ABV; {(1S,4R)-4-[2-amino-6-(cyclopropylamino)-9H-purin-9-yl]cyclopent-2-en-1-yl}methanol) and 300 mg 3TC once daily; xiii) zidovudine (Retrovir; AZT or azidothymidine) in an amount of 200 mg three times daily; xiv) Trizivir in an amount of 150 mg 3TC and 300 mg ABV and 300 mg AZT twice daily; xv) Truvada in an amount of 200 mg emtricitabine and 300 mg tenofovir (({[(2R)-1-(6-amino-9H-purin-9-yl)propan-2-yl]oxy}methyl)phosphonic acid) once daily; xvi) didanosine (Videx; 2′,3′-dideoxyinosine) in an amount of 400 mg once daily; xvii) tenofovir (Viread) in an amount of 300 mg once daily; xviii) abacavir (Ziagen) in an amount of 300 mg twice daily; xix) atazanavir (Reyataz; methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate) in an amount of 300 mg once daily or 400 mg once daily; xx) lamivudine (Epivir) in an amount of 150 mg twice daily; xxi) stavudine (Zerit; 2′-3′-didehydro-2′-3′-dideoxythymidine) in an amount of 40 mg twice daily; xxii) delavirdine (Rescriptor; N-[2-({4-[3-(propan-2-ylamino)pyridin-2-yl]piperazin-1-yl}carbonyl)-1H-indol-5-yl]methanesulfonamide) in an amount of 400 mg three times daily; xxiii) efavirenz (Sustiva; (4S)-6-chloro-4-(2-cyclopropylethynyl)-4-(trifluoromethyl)-2,4-dihydro-1H-3,1-benzoxazin-2-one) in an amount of 600 mg once daily); xxiv) nevirapine (Viramune; 11-cyclopropyl-4-methyl-5,11-dihydro-6H-dipyrido[3,2-b:2′,3′-e][1,4]diazepin-6-one) in an amount of 200 mg twice daily); xxv) bevirimat; and xxvi) Vivecon.

### Kits, Containers, Devices, Delivery Systems

Kits with unit doses of the active agent, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the drugs in treating an immunodeficiency virus (e.g., an HIV) infection. Suitable active agents (a subject interfering construct or a subject interfering particle) and unit doses are those described herein above.

In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present invention provides a delivery system comprising an active agent. In some embodiments, the delivery system is a delivery system that provides for injection of a formulation comprising an active agent subcutaneously, intravenously, or intramuscularly. In other embodiments, the delivery system is a vaginal or rectal delivery system.

In some embodiments, an active agent is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of a subject active agent. For example, a subject delivery device comprises an injection device pre-loaded with a single dose of a subject active agent. A subject injection device can be re-usable or disposable.

Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The present invention provides a delivery system for vaginal or rectal delivery of an active agent to the vagina or rectum of an individual. The delivery system comprises a device for insertion into the vagina or rectum. In some embodiments, the delivery system comprises an applicator for delivery of a formulation into the vagina or rectum; and a container that contains a formulation comprising an active agent. In these embodiments, the container (e.g., a tube) is adapted for delivering a formulation into the applicator. In other embodiments, the delivery system comprises a device that is inserted into the vagina or rectum, which device includes an active agent. For example, the device is coated with, impregnated with, or otherwise contains a formulation comprising the active agent.

In some embodiments, the vaginal or rectal delivery system is a tampon or tampon-like device that comprises a subject formulation. Drug delivery tampons are known in the art, and any such tampon can be used in conjunction with a subject drug delivery system. Drug delivery tampons are described in, e.g., U.S. Pat. No. 6,086,909. If a tampon or tampon-like device is used, there are numerous methods by which an active agent can be incorporated into the device. For example, the active agent can be incorporated into a gel-like bioadhesive reservoir in the tip of the device. Alternatively, the active agent can be in the form of a powdered material positioned at the tip of the tampon. The active agent can also be absorbed into fibers at the tip of the tampon, for example, by dissolving the active agent in a pharmaceutically acceptable carrier and absorbing the drug solution into the tampon fibers. The active agent can also be dissolved in a coating material which is applied to the tip of the tampon. Alternatively, the active agent can be incorporated into an insertable suppository which is placed in association with the tip of the tampon.

In other embodiments, the drug delivery device is a vaginal or rectal ring. Vaginal or rectal rings usually consist of an inert elastomer ring coated by another layer of elastomer containing an active agent to be delivered. The rings can be easily inserted, left in place for the desired period of time (e.g., up to 7 days), then removed by the user. The ring can optionally include a third, outer, rate-controlling elastomer layer which contains no active agent. Optionally, the third ring can contain a second active agent for a dual release ring. The active agent can be incorporated into polyethylene glycol throughout the silicone elastomer ring to act as a reservoir for drug to be delivered.

In other embodiments, a subject vaginal or rectal delivery system is a vaginal or rectal sponge. The active agent is incorporated into a silicone matrix which is coated onto a cylindrical drug-free polyurethane sponge, as described in the literature.

Pessaries, tablets, and suppositories are other examples of drug delivery systems which can be used in the context of the present disclosure. These systems have been described extensively in the literature.

Bioadhesive microparticles constitute still another drug delivery system suitable for use in the context of the present disclosure. This system is a multi-phase liquid or semi-solid preparation which does not seep from the vagina or rectum as do many suppository formulations. The substances cling to the wall of the vagina or rectum and release the drug over a period of time. Many of these systems were designed for nasal use but can be used in the vagina or rectum as well (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

### Subjects Suitable for Treatment

The methods of the present disclosure are suitable for treating individuals who have an immunodeficiency virus infection, e.g., who have been diagnosed as having an immunodeficiency virus infection. The methods of the present disclosure are also suitable for use in individuals who have not been diagnosed as having an HIV infection (e.g., individuals who have been tested for HIV and who have tested negative for HIV; and individuals who have not been tested), and who are considered at greater risk than the general population of contracting an HIV infection (e.g., “at risk” individuals).

The methods of the present disclosure are suitable for treating individuals who have an HIV infection (e.g., who have been diagnosed as having an HIV infection), and individuals who are considered at greater risk than the general population of contracting an HIV infection. Such individuals include, but are not limited to, individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals. Individuals suitable for treatment include individuals infected with, or at risk of becoming infected with, HIV-1 and/or HIV-2 and/or HIV-3, or any variant thereof.

### Methods of Generating a Variant

The present disclosure provides a method of generating a variant interfering, conditionally replicating, HIV construct. The method generally involves: a) introducing an interfering construct as described above into a first individual; b) obtaining a biological sample from a second individual to whom the interfering construct has been transmitted from the first individual (either directly or via one or more intervening individuals), wherein the construct present in the second individual is a variant of the interfering construct introduced into the first individual; and c) cloning the variant construct from the second individual.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Design Considerations for Interfering Particles

In this study, the likely direction of HIV-1 and DIP evolution within an infected individual was calculated to determine if DIP-based therapies would face an evolutionary disadvantage. Specifically, it was asked whether two mechanisms of competition corresponding to two points in the viral replication cycle—‘cis stealing’ (e.g., competition between single-stranded HIV genomic RNA and single-stranded DIP RNA to bind a second strand of HIV genomic RNA) and ‘trans stealing’ (e.g., competition for viral capsids)—are evolutionarily stable strategies of interference. The practical aim of this inquiry is to direct the design of future DIP vectors. Testing stability in cell culture is not a trivial matter, because the total number of cells, even in large flasks, is several logs lower than in an animal/human host, and the speed of evolution may be sensitive to rare double or triple mutations. Therefore, before attempting expensive long-term animal studies, it is of importance to test evolutionary stability in silico.

To do this, a model of HIV-DIP interaction was considered at two scales: (i) on the single-cell scale, a simplified intracellular model approximates the molecular mechanisms of HIV-1 particle formation and DIP formation (two models, for each type of interference), and, (ii) on the individual-patient scale, a standard model of HIV-1 dynamics in individuals (59, 64) is extended to include the presence of DIP. The fitness effect of mutation at the within-host scale (“effective selection coefficient”) was derived directly from the underlying intracellular model and it was tested whether HIV-1 is likely to evolve to decrease DIP-mediated stealing of its products and hence escape suppression by the DIP.

Supplemental information (including methods) is provided at the end of Example 1.

### Theory

### Preliminary Analysis: DIP Interference by Competition for HIV-1 Genomic RNA Leads to Divergent Evolution at the Single-Cell Level

We begin by analyzing the stability of DIPs that interfere via binding to genomic RNAs (gRNA) of the wild-type lentivirus (i.e., genome stealing). Lentiviruses are diploid and gRNAs are packaged into virions in pairs, where encapsidation of two copies of RNA is achieved by allowing the gRNAs to dimerize. This gRNA pairing is initiated at a six-nucleotide palindrome termed the dimerization initiation signal (DIS) which is located within stem loop 1 (SL1) of the HIV-1 genome and has the consensus sequence GCGCGC (55).

A minimal mathematical model that considers only non-dimerized and dimerized genomes in (FIG. 1a,b) is used to describe gRNA pairing and to analyze how the gRNA pairing would co-evolve for a DIP and HIV-1. We assume that the DIP provirus is expressed only in cells infected with both DIP and HIV-1 (dually infected cells), because, in the absence of HIV-1, DIP lacks transactivators. The model describes homozygous pairing of HIV-1 genomes (g) and DIP genomes (g_{DIP}) as well as the heterozygous pairing between DIP genomes and HIV-1 in dually infected cells. The model captures experimental evidence demonstrating that sub-genomic RNAs that share HIV-1's consensus DIS palindrome can dimerize to gRNA HIV-1 genomes (10, 55) and that partitioning of diploid genomes between homozygous and heterozygous virions is binomial (10). Importantly, heterozygous virions that contain one copy of HIV-1 genome and one copy of DIP genome (sub-genomic) are largely nonviable (2).

Furthermore, the model includes recent evidence that the overwhelming majority of HIV-1 infected cells harbor a single integrated HIV-1 provirus (36), most likely, due to the short lifetime of infected cells (25, 28) and molecular restrictions to super-infection such as Nef-downregulation of surface CD4 (25). However, the model does not restrict DIP proviral integrations to a single copy, since multiple infections of the cell require expression of trans elements, which the DIP would lack. Specifically, here we consider a DIP that does not to express Nef or any other trans elements responsible for superinfection protection. Multiple copies of integrated DIP provirus, whose number we denote m (m=1, 2, 3, . . . ), lead to the DIP gRNA monomers being more abundant than HIV-1 gRNA in the cytoplasm of the cell by a factor m, which varies among cells, and is determined in part by the abundance of circulating virus in the body (i.e., viral dynamics). In addition, we assume that DIP genome architecture, e.g., the lack of splicing sites (13, 41), leads to an expression asymmetry between DIP and HIV, such that DIP monomers are more abundant in the cytoplasm of the cell by an additional fixed factor, which we denote P (where P>1). This “expression asymmetry” has been observed for lentiviral vectors (2, 6, 13, 14, 41), including HIV-1-derived lentiviral vectors (2, 6). The enrichment of the DIP gRNA over HIV gRNA in cytoplasm is a product of both m and P. Due to enrichment of the DIP, most HIV-1 genomic RNA copies in the cell are stolen by DIP to produce non-viable virions with HIV-DIP heterodimers thereby generating a mechanism of interference.

To examine the evolutionary stability of interference by genome stealing (i.e., heterodimerization) we first consider single-residue mutations in the HIV-1 DIS (e.g., GCGCGC→GCGAGC). Such mutations lead to mismatches and decreases in the probability of heterodimerization but result in more severe decreases in HIV-1 homodimerization (FIG. 1c). However, compensatory double-residue mutations reestablish a new DIS palindrome (e.g., GCGCGC→GCUAGC) and generate a situation in which DIP-to-HIV-1 heterozygous dimerization is far less favorable than homozygous dimerization (FIG. 1c). By this logic, the idealized model that considers dimerization coefficients to be only a function of the number of mismatches leads to divergent evolution of the DIS sequences between HIV-1 and DIP due to double mutations in HIV-1 DIS (FIG. 1d). Although a single mutation in HIV-1's DIS is more deleterious to HIV-1 homodimerization than to DIP-to-HIV-1 heterodimerization, a second mutation within the DIS will rescue HIV-1 dimerization and generate a further decrease in DIP-to-HIV-1 genome stealing.

This analysis does not explicitly account for the cost of the palindrome DIS being different than the consensus GCGCGC sequence since different HIV-1 strains encode different DIS palindromes (55). Another limitation of this analysis is that it only considers a single round of mutation. However, these assumptions are not critical for the conclusion of evolutionary instability of genome stealing. More realistic models, capturing multiple rounds of mutation still lead to progressive decreases in heterodimerization relative to homodimerization (FIG. 5).

FIGS. 1A-D.

Divergent evolution of the HIV-1 and DIP dimerization initiation sequences (DIS) by double mutations in HIV-1 indicates that DIP interference by “genome-stealing” is evolutionary unstable. (a) Schematic showing that genomic RNA (gRNA) monomers of HIV-1 and DIP form three types of dimer complexes (HIV-HIV, HIV-DIP, and DIP-DIP) based upon a “kissing loop” formation between the dimerization initiation sequences of HIV-1 and DIP, which contain a palindromic sequence (e.g., the consensus sequence GCGCGC). Due to a faster rate of transcription and multiple provirus copies, DIP monomers are more abundant, so that most of HIV-1 RNA is wasted on non-viable HIV-DIP heterodimers. (b) A simplified model representing the abundance of gRNA monomers for HIV-1 and DIP in the cytoplasm of the infected cell, g(t) and g_{DIP}(t), respectively. θ is a lumped parameter representing the linear rate of gRNA production and P is the expression asymmetry between HIV-1 and DIP. k_{H}, k_{IP}, and k_{HIP} are dimerization coefficients for HIV-HIV, DIP-DIP, and HIV-DIP, respectively. (c) Potential mutations in the kissing loop lead to divergent evolution of HIV-1 and DIP. Top row: In the ‘wild-type’ (wt) HIV-1 and DIP case, there is an exact match for any gRNA pair (HIV-HIV, HIV-DIP, and DIP-DIP), which is enumerated in the rightmost column. Middle rows: If a single mutation arises within HIV-1 (highlighted by the green rectangle), HIV-HIV homodimers have two mismatches compared to HIV-DIP heterodimers which have only a single mismatch and DIP-DIP homodimers which have no mismatches. Bottom rows: In the (likely) scenario where the second compensatory mutation occurs in HIV, heterodimerization is disfavored compared to homodimerization. (d) Evolutionary ‘fitness’ of homodimers and heterodimers qualitatively estimated based on dimerization coefficients in panel B and the number of sequence matches in panel C. In this idealized model, fitness takes the canonical functional form of an exponential where the selection coefficient, s, is based only upon the dimerization coefficients and the degree of sequence matching. Although a single mutation in HIV-1's DIS is more deleterious to HIV-1 homodimerization than to DIP-to-HIV-1 heterodimerization, a second mutation within the DIS will rescue HIV-1 dimerization and generate a further decrease in DIP-to-HIV-1 genome stealing.

### An Alternative Mechanism of Interference: Capsid Stealing by DIP

Given that interference by genome stealing is unstable (see above), we next considered whether interference by DIP competition for trans elements, such as the capsid, was evolutionarily stable. Since DIPs carry the full complement of cis elements but lack one or more trans elements (e.g., capsid, envelope), DIPs must rely on the trans elements of the wild-type lentivirus to package and mobilize out of the infected cell. Thus, DIPs compete for and parasitize trans elements leading to interference with wild-type virus.

To calculate DIP interference on HIV-1 viral loads and the associated evolutionary stability, we use a recently developed multi-scale modeling approach (53) to integrate a single-cell model of competition for capsid with an individual-patient model. Unlike previous multi-scale modeling of virus infections (29, 30, 42), this multi-scale model is designed specifically for HIV-1 in the presence of a DIP. The model applies to a range of DIP vectors, from a minimal DIP that does not code for any trans elements, to a DIP that codes for tat and rev (allowing for expression and export of genomes to cytoplasm even in the absence of HIV-1 provirus), but does not express capsid proteins or Nef (which mediates superinfection protection). In the following subsections, we describe the single-cell and individual-host models, their integration, and their parameters. In the last subsection, we introduce the notion of “effective selection coefficient”. Detailed analytical derivations are presented in Supplemental Methods (below). Numerical solutions for all figures were performed in MATLAB™ (version R2011a).

The Single-Cell Model with Capsid Stealing

For tractability, the single-cell model considered here (FIG. 2a) is simplified and considers only intracellular replication events relating to dimerized wild-type HIV-1 RNA genomes (G), encapsidation-competent capsid (C), and dimerized DIP RNA genomes (G_{DIP}). The equations have the form:

$$\begin{matrix}\frac{d G}{d t} = \underbrace{θ}_{\begin{matrix}HIV genome \\ production\end{matrix}} - \underbrace{{k}_{pck} GC}_{\begin{matrix}Packaging of \\ HIV genomes \\ into capsids\end{matrix}} - {\underbrace{α G}}_{\begin{matrix}Loss of \\ HIV genomes\end{matrix}} & ( 1 ) \\ \frac{d C}{d t} = \underbrace{ηθ}_{\begin{matrix}Capsid \\ production\end{matrix}} - \underbrace{{k}_{pck} ( G + {G}_{DIP} ) C}_{Encapsidation of genomic RNSs} - \underbrace{β C}_{\begin{matrix}Loss of \\ capsids\end{matrix}} & ( 2 ) \\ \frac{d {G}_{DIP}}{d t} = \underbrace{mP θ}_{\begin{matrix}DIP genome \\ production\end{matrix}} - \underbrace{{k}_{pck} {G}_{DIP} C}_{\begin{matrix}Packaging of \\ DIP genomes \\ into capsids\end{matrix}} - \underbrace{α {G}_{DIP}}_{\begin{matrix}Loss of \\ DIP genomes\end{matrix}} & ( 3 )\end{matrix}$$

Model parameters are defined in Table 1. Briefly, the model describes the production and decay of dimerized HIV-1 genomes, the packaging of these dimerized genome into capsids that are produced at a rate proportional to genome dimers, and the competition for encapsidation between DIP genomes and HIV-1 genomes. The model neglects heterozygous genomes since, as demonstrated above, the dimerization initiation sequence for HIV-1 and the DIP will diverge so that heterozygous pairing is eliminated. As in the genome-stealing model (above), we allow each DIP provirus to express more RNA than an HIV-1 provirus by a fixed factor of P (2, 6, 13, 14, 41). This expression asymmetry (P>1) is due to differences in genome architecture, such as mutated alternative-splicing sites (13, 41). We classify dually infected cells by the number of DIP provirus copies m (m=1, 2, 3 . . . ). The net asymmetry between DIP and HIV-1 expression in a dually infected cell is the product m·P. Based on previous results (53), we assume that the HIV-1 and DIP levels inside of cells rapidly reach steady state; the steady state values of G, G_{DIP}, and C are derived analytically in Supplemental Methods (Equations S6-S14).

TABLE 1
State variables and model parameters for intracellular capsid-stealing
model (FIG. 2A and Equations 1-3 in Theory)
State variables

| Notation | Definition | [Units] |
|---|---|---|
| G | Concentration of full-length dimerized HIV genomic mRNAs | [1/μL] |
| G_{DIP} | Concentration of full-length dimerized DIP genomic mRNAs | [1/μL] |
| C | Concentration of encapsidation-competent capsids in cytoplasm | [1/μL] |
| m | DIP (integrated) provirus copy # (i.e., MOI) | dimensionless |

Model parameters

| Notation | Definition | Units | Value | Ref. |
|---|---|---|---|---|
| θ k_{pck} α β | Rate of accumulation of encapsidation-competent HIV genomes in cytoplasm Packaging constant Loss of genome rate Loss of capsid rate | [1/μL/day] [μL/day] [1/day] [1/day] | $\begin{matrix}\begin{matrix}\begin{matrix}\begin{matrix}\begin{matrix} \\ \end{matrix} \\ \end{matrix} \\ \end{matrix} \\ \end{matrix} \\ \end{matrix} \} κ = \frac{αβ}{θ {k}_{pck}} vary$ | |
| η | Capsid-to-genome | dimensionless | 1.2-5 | (10, 72) |
| | accumulation ratio | | | |
| P | Expression asymmetry | dimensionless | 8-10 | (13) |
| | between DIP and HIV | | | |

### The Individual-Host Model

Similar to the method previously used (53), output of the single-cell model is used to calculate HIV-1 and DIP viral loads within an individual patient using a standard model of HIV-1 in vivo dynamics (25, 59, 65) that is generalized to include production of DIP particles. The generalized model includes co-infection of cells with DIP and HIV-1 so that dually infected cells produce less HIV-1. Based on the results of recent in vivo studies (36), we assume a single HIV-1 provirus per (singly or dually) infected cell but allow for multiple DIP provirus copies per cell, as previously proposed (80). Briefly, the model describes: T, uninfected CD4⁺ T cells permissive for viral replication; I, cells infected with HIV-1 only; T_{DIP m}, CD4⁺ T cells harboring m copies of DIP provirus but not infected with HIV-1 (by definition, T_{DIP 0}=T); I_{D m}, dually infected cells harboring a copy of HIV-1 and m copies of DIP provirus; V, HIV-1 load (free virus concentration in peripheral blood plasma); V_{DIP}, DIP load. The system of equations has the form:

$$\begin{matrix}\frac{d T}{d t} = b - ( d + kV + {kV}_{DIP} ) T & ( 4 ) \\ \frac{d I}{d t} = kVT - δ I & ( 5 ) \\ \frac{d {T}_{DIP m}}{d t} = {kV}_{DIP} {T}_{DIP m - 1} - ( d + kV + {kV}_{DIP} ) {T}_{DIP m} , \\ m = 1 , 2 , 3 , … & ( 6 ) \\ \frac{d {I}_{D m}}{d t} = {kVT}_{DIP m} - δ {I}_{D m} , m = 1 , 2 , 3 , … & ( 7 ) \\ \frac{d V}{d t} = n δ I + n δ {\sum }_{m = 1}^{∞} {ψ}_{m} {I}_{D m} - cV & ( 8 ) \\ \frac{d {V}_{DIP}}{d t} = n δ {\sum }_{m = 1}^{∞} {ρ}_{m} {ψ}_{m} {I}_{D m} - {cV}_{DIP} & ( 9 )\end{matrix}$$

The model parameters, which are well described in the literature and summarized in Table 2, are: b, the linear production rate of uninfected cells; d, the natural death rate of uninfected cells; k, the infectivity factor; δ, the death rate of singly and dually infected cells; n, the HIV-1 burst size from a singly infected cell. There are two additional parameters in the presence of DIP: ψₘ, the ratio of HIV-1 burst size between a singly infected cell, I, and a dually infected cell with m copies of DIP provirus I_{D m}, and ρₘ, the ratio of DIP to HIV-1 burst size from a dually infected cell with m copies of DIP provirus.

TABLE 2
State variables and parameters for the individual-host model
(Equations 4-9 in Theory)
State variables

| Notation | Definition | Units |
|---|---|---|
| T | Uninfected CD4⁺ T cells permissive for viral replication | [cells/μL] |
| I | CD4⁺ T cells infected with HIV only | [cells/μL] |
| T_{DIP m} | CD4⁺ T cells infected with m copies of DIP provirus but not | [cells/μL] |
| | infected with HIV (T_{DIP 0} = T) | |
| I_{D m} | Dually infected cells with an HIV and m copies of DIP | [cells/μL] |
| | provirus | |
| V | HIV viral load | [RNA copies/] |
| V_{D} | DIP viral load | [RNA copies/mL] |

Model parameters

| Notation | Definition | Units | Value | Ref. |
|---|---|---|---|---|
| β | Linear production rate of uninfected cells | [cells/μL/day] | ${R}_{0} = \frac{bkn}{cd} ∼ 10$ | (60) |
| d | Death rate of uninfected cells | [1/day] | | |
| k | Infectivity factor | [mL/day/ | | |
| | | RNA copy] | | |
| n | HIV burst size from a singly infected | [RNA copy/cell] | | |
| | cell | | | |
| c | Virion clearance rate | [1/day] | | |
| δ | Death rate of HIV infected cells | [1/day] | 1.0/day | (50) |
| nψₘ | HIV burst size from a dually infected | dimensionless | k_{pck}GC/δ | From |
| | cell with m copies of DIP provirus | | | single- |
| nρₘψₘ | DIP burst size from a dually infected | dimensionless | k_{pck}G_{DIP}C/δ | cell |
| | cell with m copies of DIP provirus | | | model |

The steady states of the single-cell model (Equations 1-3) define the following burst sizes for HIV-1 and DIP (Supplementary Methods; Equations S6-S20):

n=k_{pck}[GC]_{P=0}/δ (10)

ψₘn=k_{pck}GC/δ (11)

ρₘψₘn=k_{pck}G_{DIP}C/δ (12)

These expressions serve as input parameters for the individual-host model. The individual-host model (Equations 4-9) is similar to the model in (53), except that here we relax the restriction of a single DIP copy per cell and allow cells to have multiple DIP infections. We assume that the state variables of the individual-host model (Equations 4-9) are in steady state, which corresponds to chronic infection. Equations 4-9 are used to calculate steady state levels, as described in Supplementary Methods (Equations S34-S43 and the following subsection).

### Parameter Values

The full list of model parameters is given in Tables 1 and 2. Using a standard approach, we reduce the number of parameters to a smaller number of composite parameters by changing units to those in which the state variables are measured (Supplemental Methods). As a result of this non-dimensionalization, all results can be conveniently expressed in terms of two non-dimensional parameters that capture the evolutionary potential of HIV-1: (i) the composite ‘waste’ parameter κ=(αβ)/(θk_{pck}), which reflects the loss of HIV-1 genomes (rate α, Equation 1) and capsids (rate β, Equation 2), and (ii) the ratio of encapsidation-competent capsids to dimerized HIV-1 genomes produced per unit time (η) (referred to as the “capsid-to-genome production ratio”). The remaining parameters are determined from the basic reproductive ratio (R₀) which is estimated from in vivo data as R₀˜10 (see Ref. (60), Table 3, the case of an exponentially distributed production delay with the average between 24 h and 12 h), and by using fixed values of P. Lentiviral DIPs with P=8-10 have been engineered (14). We focus on the conservative value P=5 below, but the interval P=2-30 is also studied (see Supplementary Information).

### Testing Evolutionary Stability: The Effective Selection Coefficient

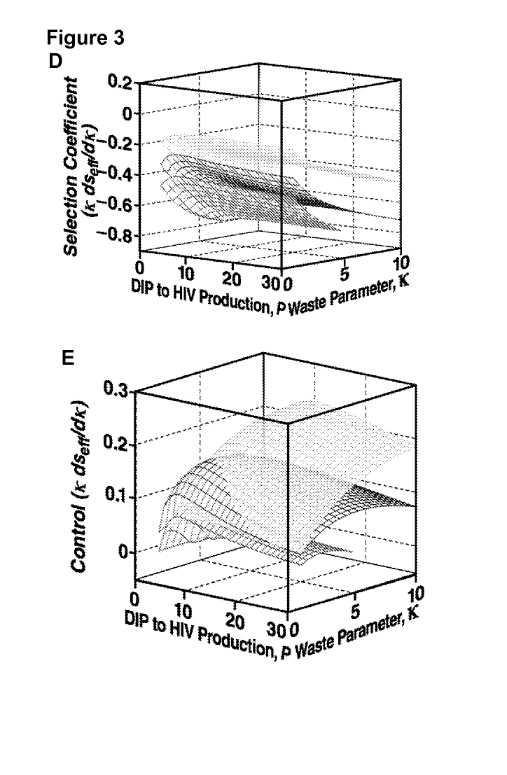

To determine if HIV-1 mutates to increase or decrease capsid waste (or capsid-genome rate ratio), we capitalized on previous studies that applied concepts from Darwinian evolution, based on the notion of the “selection coefficient” (s), to analyze the evolution of single-locus mutations in the HIV-1 genome (11, 68). Negative values of s denote that a mutation decreases the progeny of an infected cell and is selected against, and positive values of s denote that a mutation is selected for. Calculating s is complicated by the compartmental structure of a population of infected cells that includes both dually (HIV+DIP+) and singly (HIV+DIP−) infected cells that may contribute differently to the fitness effect of mutation (number of progeny). Calculation of s is also affected by the dynamic interaction of HIV-1 and DIP within dually infected cells. To account for these complexities, we introduce the “effective selection coefficient” (∂s_{eff}) defined as the exponential rate of increase, or decrease, of mutant virus normalized to the death rate of infected cells. To calculate ∂s_{eff}, we begin with a DIP-HIV-1 co-infection at steady state and perturb the system by adding a small amount of mutant HIV-1. In particular, we consider mutations that increase capsid waste (κ→κ+∂κ, ∂κ>0), achieved by slightly reducing k_{pkg} (FIG. 3a). We then calculate the expansion (contraction) rate of the mutant subpopulation from the model equations (Equations 1-12) and arrive at normalized value ∂s_{eff}/(∂κ/κ). The normalized selection coefficient for mutations affecting the capsid-to-genome production ratio η is calculated in analogous fashion. Detailed derivations are given in Supplemental Methods; Equations S48-57.

### Results

### Evolutionary Stability of Capsid-Stealing Interference: General Approach

Our preliminary analysis (see Theory, Preliminary Analysis) showed that the genome-stealing mechanism of DIP interference with HIV-1 replication is evolutionarily unstable due to divergence of the HIV-1 and DIP dimerization initiation sequences (FIG. 1). Here, we determine whether interference by DIP competition for trans elements, such as capsid protein, leads to stable and sustained interference. We use our recently developed multi-scale modeling approach to integrate a single-cell model with an individual-patient model (53). The single-cell model (Equations 1-3) captures the reported ability of minimal lentiviral vectors to express more RNA in cytoplasm compared to HIV-1 (2, 6, 13, 14, 41), and this “expression asymmetry” is represented by the parameter P (where P>1). The output from this single-cell model (i.e., DIP and HIV-1 burst sizes) is used as input for the individual-patient model (Equations 4-9, Table 2)—a generalized form of the standard model of HIV-1 in vivo dynamics (25, 59, 65) that includes production of DIP particles—to ultimately calculate HIV-1 and DIP viral loads within a patient (FIG. 2A). Detailed analytical derivations and numeric calculations are presented in Supplemental Methods.

DIP Interference by Competition for Capsid within Single Cells Leads to Sustained Suppression of HIV-1 at the Individual-Patient Level

To test whether DIP lowers HIV-1 viral load, we examined the steady-state values of HIV-1 and DIP at the individual-patient scale as a function of parameters in the single-cell model. As detailed above, the results are expressed in terms of a pair of intuitive and non-dimensional parameters that capture the evolutionary potential of HIV-1 and DIP: the composite ‘waste’ parameter (κ), and the ratio of encapsidation-competent capsids to dimerized HIV-1 genomes produced per unit time (η).

Analytical solutions demonstrate that HIV-1 viral load is stably decreased across a broad range of κ values (FIG. 2b, red lines). In most cases, viral load is decreased by more than one order of magnitude, which is associated with significantly reduced HIV-1 transmission and disease progression (21). Stable DIP-mediated suppression of HIV-1 depends upon the expression asymmetry P>1 (53, 80) and, importantly, is amplified by the large average multiplicity of DIP infection (see next section and FIG. 7a). However, the decrease in HIV-1 viral load is not completely due to DIP and is partly due to the loss of HIV-1 capsids and genomes by increased capsid waste (black dotted lines). Nevertheless, DIP-mediated suppression contributes to a significant fraction of this decrease through stealing HIV-1 capsids within dually-infected cells and through competition for available target cells within the infected individual (FIG. 6). At high κ, there is a loss of dynamic stability because at such high capsid waste, HIV-1 crosses the threshold of its own extinction.

Even at modest values of expression asymmetry P, the high multiplicity of DIP infection generates relatively high DIP viral loads (see next section and FIG. 7) and allows the DIP to be dynamically stable even when η is just larger than 1 (FIG. 2c).

Thus, DIPs that steal capsids can stably suppress HIV-1 viral load even if the DIP's expression asymmetry, P, is modest. These results are robust and qualitatively similar across a broad range of P and η values (FIG. 2d-e).

FIGS. 2A-E.

DIPs that steal capsid stably suppress HIV-1 load across a broad range of parameters. (a) Schematic of the model comprising two scales of biological organization. The in vivo (individual host) scale is the standard model of HIV-1 replication expanded to include DIPs (see Supplemental Methods; Equations S28-S33). Uninfected cells can be infected with either HIV-1 or DIP; DIP⁺ cells can be superinfected with HIV-1 to become dually infected cells. The single-cell model is described by Equations 1-3. A dually infected cell has one integrated HIV-1 provirus and multiple, m, copies of DIP provirus. A fraction of HIV-1 gRNA is translated into proteins that form ‘empty’ capsids. DIP does not express proteins. Dashed arrows represent multi-stage processes (including the loss of RNA monomers and capsid proteins). A fraction of stable dimer genomes and full capsids is also lost. Remaining genomes, HIV-1 or DIP, are packaged within capsids and released as infectious particles. (b) Steady-state HIV-1 load and (c) steady-state DIP load at different values of two single-cell parameters: the capsid ‘waste’ parameter, κ, and the capsid-to-genome production ratio, η(see Table 1). The dashed line shows HIV-1 viral load in the absence of capsid waste and DIP (κ=P=0), which is assumed to be the average load in untreated humans (3·10⁴ RNA copies/ml blood). Calculations use a DIP:HIV-1 production ratio (i.e., expression asymmetry) of P=5 and a basic reproduction ratio of R₀=10 (Table 1). The decrease in HIV-1 load in the presence of capsid waste (κ>0, red lines), as compared to the untreated HIV-1 ‘set-point’ level (dashed line), is partly due to the loss of HIV-1 products (black dotted lines calculated at P=0) and partly due to DIP, which competes with HIV-1 for available target cells and steals HIV-1 capsid in dually infected cells. The first effect is more important at η=˜1, and the DIP suppression factor is stronger at large η (see Supplemental Material FIG. 6). (d) Steady-state HIV-1 load and (e) steady-state DIP load as a function of both expression asymmetry, P, and capsid waste parameter, κ, at three values of capsid-to-genome ratio, η=2 (red), η=5 (green), η=10 (blue). These 3D plots act as a partial sensitivity analysis showing that HIV-1 and DIP loads depend strongly on P.

### Robust Suppression of HIV-1 is Due to High Multiplicity of DIP Infection

As previously noted (80), DIP-infected cells would not be restricted to harboring only a single DIP provirus since DIP-infected cells are long lived and could be readily re-infected multiple times by DIP before HIV-1 infects the cell. DIP super-infection will lead to multiple integrated DIP genomes per cell. The number of DIP copies (denoted m) varies among dually infected cells as predicted by the individual-patient model (Theory, Equations 4-9). We calculated the average DIP copy number, denoted E[m], as a function of capsid waste (κ) and expression asymmetry (P) for different values of the capsid-to-genome ratio (η) (Supplemental Methods and FIG. 7a). The result is dependent upon P in a positive manner: increases in P leads to more DIP virions, which results in greater multiplicity of DIP infection. It is useful to note that, according to the standard individual-patient model we use here, the probability that a DIP+HIV− cell is infected with another DIP copy is proportional to the concentration of DIP virions. Dually infected ‘producer’ cells contain higher levels of DIP gRNA than HIV-1 gRNA (i.e., P>1) and these cells generate more DIP than HIV-1 virions, which results in an increase of average DIP copy number with P. Thus, the ratio of DIP:HIV-1 gRNA within a cell is determined by P in two ways: directly (through the molecular architecture of the DIP) and indirectly through the increase in multiplicity of DIP infection (m).

To further explore the contribution of m to HIV-1 interference and suppression, we artificially limited DIP multiplicity to a single copy per cell (i.e., m=1 or m=0) and recalculated the HIV-1 viral loads (FIG. 7b). This ‘control’ demonstrates that when m≦1, DIP-mediated suppression contributes very little to the decrease in HIV-1 viral load (i.e., when m≦1, HIV-1 suppression is modest and arises primarily due to increased capsid waste). The analysis further shows that, when m≦1 the DIP loses stability at low η as κ increases. Hence, when m≦1, high η is needed for even modest suppression of HIV-1 viral load. In summary, the multiplicity of integrated DIP genomes (m) is critical for DIP-mediated suppression of HIV-1.

### Capsid Interference is Evolutionarily Stable

We next examined the direction of evolution of the DIP and HIV-1 to test whether the DIP would be selected against in an ongoing HIV-1 infection. Conceivably, HIV-1 could escape DIP by mutating to effectively increase the capsid waste parameter (κ). One possible mechanism to increase κ is for HIV-1 to mutate its packaging signal Ψ and thus decrease packaging efficiency allowing more genomes, or capsids, to be degraded instead of packaged. Under this increased-waste scenario, capsid stealing by DIP would be more affected than HIV-1 packaging in dually infected cells due to the DIP expression asymmetry (P>1) and integration multiplicity (m>1). Thus, increased capsid waste would benefit HIV-1 due to a decrease in DIP interference. However, in the absence of DIP, mutation toward increased capsid waste would be deleterious for HIV-1 since increased loss of capsid products lowers the HIV-1 burst size. With these competing pressures, it is not clear which evolutionary direction dominates. These competing effects of mutation in the packaging loop are in fact evident from the steady state HIV-1 load versus κ (FIG. 2b) where there are two components of HIV suppression: one due to decrease in HIV-1 burst (black dotted curves versus dashed line in FIG. 2b), and another due to DIP interference (red curves versus black dotted curves in FIG. 2b). As κ is increased, one component becomes larger and another smaller. It is not immediately clear which effect is stronger.

To determine if HIV-1 mutates to increase or decrease capsid waste, we calculate the “effective selection coefficient” (∂s_{eff}), defined as the exponential rate of increase, or decrease, of mutant HIV-1 normalized to the death rate of infected cells (Theory and Supplemental Methods, Equations S48-57). Negative values of s_{eff} denote that a mutation decreases the progeny of an infected cell and is selected against, while positive values of s_{eff} denote that a mutation is selected for. We consider mutations that slightly increase κ by reducing the packaging efficiency (FIG. 3a). Because the effect of mutation on κ, denoted ∂κ, is unknown and may vary among bases, the selection coefficient is expressed in a normalized form ∂s_{eff}/(∂κ/κ). Unlike previous work, this study is unique in that it calculates an in vivo selection-coefficient value directly from a molecular model. Previous studies were only able to estimate s by fitting (4, 23, 26, 31, 57, 68, 79).

Analyzing the effective selection coefficient as a function of capsid waste (κ) demonstrates that, overall, HIV-1 mutants with increased capsid waste are selected against since ∂s_{eff}/(∂κ/κ)<0 for a range of η and κ (FIG. 3b). Importantly, HIV-1 mutants with high capsid waste are selected against despite DIP interference being decreased at high capsid waste (see FIG. 2b for low values of η).

As a negative control, we next examined the effective selection coefficient keeping burst size of HIV-1 in singly infected cells constant, as capsid waste increases. As expected, in this scenario HIV-1 does evolve toward high capsid waste as shown by ∂s_{eff}/(∂κ/κ) being >0 for a range of η and κ (FIG. 3c). The direction of evolutionary selection appears robust across a broad range of P values (FIG. 3d-e). Hence, it is the decrease in HIV-1 burst size that causes the negative selection coefficient in FIG. 3b.

FIGS. 3A-E.

DIP-HIV interaction is evolutionary stable over a broad parameter range: HIV-1 cannot escape DIP by decreasing packaging resources. (a) Schematic of the two-scale model for an individual infected by two strains of HIV, wild type (red) and mutant (orange), as well as DIP (blue). Mutation causes a small decrease in the packaging constant of both HIV-1 and DIP k_{pck} and, hence, an increase in capsid waste parameter κ=αβ/(θk_{pck}) when ∂κ>0. (b) Normalized effective selection coefficient ∂s_{eff}/(∂κ/κ) for that mutation, as a function of κ, for a range of capsid-to-genome production ratios η. Fixed parameters are as described in FIG. 2b,c: R₀=10, P=5. The negative values of ∂s_{eff}/(∂κ/κ) imply that the mutation has net deleterious effects on HIV-1 replication. Overall, HIV-1 mutations that increase capsid waste are selected against. Inset: HIV-1 load as a function of waste parameter from FIG. 2b. (c) A negative control showing ∂s_{eff}/(∂κ/κ) within HIV⁺DIP⁺ dually infected cells when burst-size changes due to increased capsid waste (1st term in Equation S54 in Supplemental Methods) are neglected. Only in this specific context, when burst-size changes are ignored, are HIV-1 mutations that increase capsid waste selected for. (d) Net ∂s_{eff}/(∂κ/κ) and (e) control ∂s_{eff}/(∂κ/κ) (i.e., when burst-size changes are neglected) as a function of both P and κ: These 3D plots act as a partial sensitivity analysis and show that the selection coefficient weakly depends on P. Detailed calculations are given in Supplementary Methods.

Essentially, these results indicate that the base HIV-1 burst size (which affects both singly and dually infected cells)—and not DIP interference within dually infected cells—would dominate HIV-1 evolution within the individual patient.

Next, we sought to determine if HIV-1 could escape DIPs by mutating by reducing the available capsid material, i.e., decreasing its capsid-to-genome ratio η (FIG. 4a). The analysis shows that the DIP is unstable only at low values of η and, as expected, HIV-1 evolves towards high values of η; i.e., mutants that produce more capsid are selected for since ∂s_{eff}/(∂η/η)>0 for a large range of κ and η (FIG. 4b). In fact, there is only a narrow band (1<η<(P+1)R₀/[P(R₀−1)]) where the DIP is unstable and HIV-1 replication does not evolve toward increasing values of η (in this narrow band the selection coefficient is zero). As a control, we examined keeping HIV-1 burst-size constant as η increases (1^{st} term in Equation S54 in Supplemental Methods). The control analysis shows that the effective selection coefficient is positive in the interval where DIP is stable (FIG. 4c). Finally, a partial sensitivity analysis shows that the selection coefficient depends only weakly on the values of P and the direction of evolutionary selection appears robust across a broad range of P values (FIG. 4d-e).

FIGS. 4A-E.

DIV-HIV interaction is evolutionary stable over a broad parameter range: HIV-1 cannot escape DIP by decreasing the capsid-to-genome ratio. (a) Schematic of the two-scale model for an individual infected by two strains of HIV-1, wild type (red) and mutant (orange), as well as DIP (blue). Mutation causes a small increase in the capsid-to-genome ratio η by ∂η>0. (b) Normalized effective selection coefficient ∂s_{eff}/(∂η/η) for that mutation, as a function of η, for three values of the waste parameter κ. Fixed parameters used are as described in FIG. 2b-c: R₀=10, P=5. Inset: Corresponding HIV-1 viral load as a function of η at three values of the waste parameter κ. The positive values of ∂s_{eff}/(∂η/η) imply that mutation is selected for, and HIV-1 evolves towards increasing η. When κ=0 and η<1, the DIP is not dynamically stable in in vivo and the selection coefficient is due exclusively to an increase in the HIV-1 burst size. In a narrow adjacent interval 1<η<(P+1)R₀/[P(R₀−1)], DIP is still unstable, and HIV-1 replication does not require more capsid, which is why the selection coefficient is zero. (c) A negative control neglecting HIV-1 burst-size changes due to mutation (1^{st} term in Equation S54 in Supplemental Methods) and showing that the effective selection coefficient is positive in the interval where DIP is stable. The discontinuity at κ=0 and η=1+P is due to DIP gRNA competition with HIV gRNA for capsids at η<1+P, but not at η>1+P, when there are enough capsids for both DIP and HIV. (d) Net ∂s_{eff}/(∂η/η) and (e) control ∂s_{eff}/(∂η/η) as a function of both P and η. Discontinuities at η=1+mP, m=1, 2, . . . , are analogous to the discontinuity described in panel C. These 3D plots act as a partial sensitivity analysis and show that the selection coefficient depends weakly on P.

### REFERENCES

1. Aaskov, J., K. Buzacott, H. M. Thu, K. Lowry, and E. C. Holmes. 2006. Long-term transmission of defective RNA viruses in humans and Aedes mosquitoes. Science 311:236-238.
2. An, D. S., K. Morizono, Q. X. Li, S. H. Mao, S. Lu, and I. S. Chen. 1999. An inducible human immunodeficiency virus type 1 (HIV-1) vector which effectively suppresses HIV-1 replication. J Virol 73:7671-7677.
3. Barrett, A. D., and N. J. Dimmock. 1986. Defective interfering viruses and infections of animals. Curr Top Microbiol Immunol 128:55-84.
4. Batorsky, R., M. F. Kearney, S. E. Palmer, F. Maldarelli, I. M. Rouzine, and J. M. Coffin. 2011. Estimate of effective recombination rate and average selection coefficient for HIV in chronic infection. Proc Natl Acad Sci USA 108:5661-5666.
5. Bonhoeffer, S., C. Chappey, N. T. Parkin, J. M. Whitcomb, and C. J. Petropoulos. 2004. Evidence for positive epistasis in HIV-1. Science 306:1547-1550.
6. Bukovsky, A. A., J. P. Song, and L. Naldini. 1999. Interaction of human immunodeficiency virus-derived vectors with wild-type virus in transduced cells. J Virol 73:7087-7092.
7. Cave, D. R., F. M. Hendrickson, and A. S. Huang. 1985. Defective interfering virus particles modulate virulence. J Virol 55:366-373.
8. Chattopadhyay, S. K., H. C. Morse, 3rd, M. Makino, S. K. Ruscetti, and J. W. Hartley. 1989. Defective virus is associated with induction of murine retrovirus-induced immunodeficiency syndrome. Proc Natl Acad Sci USA 86:3862-3866.
9. Chen, C. J., A. C. Banerjea, G. G. Harmison, K. Haglund, and M. Schubert. 1992. Multitarget-ribozyme directed to cleave at up to nine highly conserved HIV-1 env RNA regions inhibits HIV-1 replication—potential effectiveness against most presently sequenced HIV-1 isolates. Nucleic Acids Res 20:4581-4589.
10. Chen, J., 0. Nikolaitchik, J. Singh, A. Wright, C. E. Bencsics, J. M. Coffin, N. Ni, S. Lockett, V. K. Pathak, and W. S. Hu. 2009. High efficiency of HIV-1 genomic RNA packaging and heterozygote formation revealed by single virion analysis. Proc Natl Acad Sci USA 106:13535-13540.
11. Coffin, J. M. 1995. HIV population dynamics in vivo: implications for genetic variation, pathogenesis, and therapy. Science 267:483-488.
12. Cook, W. J., K. A. Green, J. J. Obar, and W. R. Green. 2003. Quantitative analysis of LP-BM5 murine leukemia retrovirus RNA using real-time RT-PCR. J Virol Methods 108:49-58.
13. D'Costa, J., H. Brown, P. Kundra, A. Davis-Warren, and S. Arya. 2001. Human immunodeficiency virus type 2 lentiviral vectors: packaging signal and splice donor in expression and encapsidation. J Gen Virol 82:425-434.
14. D'Costa, J., S. G. Mansfield, and L. M. Humeau. 2009. Lentiviral vectors in clinical trials: Current status. Curr Opin Mol Ther 11:554-564.
15. DePolo, N. J., C. Giachetti, and J. J. Holland. 1987. Continuing coevolution of virus and defective interfering particles and of viral genome sequences during undiluted passages: virus mutants exhibiting nearly complete resistance to formerly dominant defective interfering particles. J Virol 61:454-464.
16. DePolo, N. J., and J. J. Holland. 1986. Very rapid generation/amplification of defective interfering particles by vesicular stomatitis virus variants isolated from persistent infection. J Gen Virol 67 (Pt 6):1195-1198.
17. Dimmock, N. J. 1985. Defective interfering viruses: modulators of infection. Microbiol Sci 2:1-7.
18. Dull, T., R. Zufferey, M. Kelly, R. J. Mandel, M. Nguyen, D. Trono, and L. Naldini. 1998. A third-generation lentivirus vector with a conditional packaging system. J Virol 72:8463-8471.
19. Evans, J. T., and J. V. Garcia. 2000. Lentivirus vector mobilization and spread by human immunodeficiency virus. Hum Gene Ther 11:2331-2339.
20. Finzi, D., J. Blankson, J. D. Siliciano, J. B. Margolick, K. Chadwick, T. Pierson, K. Smith, J. Lisziewicz, F. Lori, C. Flexner, T. C. Quinn, R. E. Chaisson, E. Rosenberg, B. Walker, S. Gange, J. Gallant, and R. F. Siliciano. 1999. Latent infection of CD4+ T cells provides a mechanism for lifelong persistence of HIV-1, even in patients on effective combination therapy. Nat Med 5:512-517.
21. Fraser, C., T. D. Hollingsworth, R. Chapman, F. de Wolf, and W. P. Hanage. 2007. Variation in HIV-1 set-point viral load: epidemiological analysis and an evolutionary hypothesis. Proc Natl Acad Sci USA 104:17441-17446.
22. Frost, S. D., M. J. Dumaurier, S. Wain-Hobson, and A. J. Brown. 2001. Genetic drift and within-host metapopulation dynamics of HIV-1 infection. Proc Natl Acad Sci USA 98:6975-6980.
23. Frost, S. D., M. Nijhuis, R. Schuurman, C. A. Boucher, and A. J. Brown. 2000. Evolution of lamivudine resistance in human immunodeficiency virus type 1-infected individuals: the relative roles of drift and selection. Journal of virology 74:6262-6268.
24. Frost, S. D., T. Wrin, D. M. Smith, S. L. Kosakovsky Pond, Y. Liu, E. Paxinos, C. Chappey, J. Galovich, J. Beauchaine, C. J. Petropoulos, S. J. Little, and D. D. Richman. 2005. Neutralizing antibody responses drive the evolution of human immunodeficiency virus type 1 envelope during recent HIV infection. Proc Natl Acad Sci USA 102:18514-18519.
25. Furtado, M. R., D. S. Callaway, J. P. Phair, K. J. Kunstman, J. L. Stanton, C. A. Macken, A. S. Perelson, and S. M. Wolinsky. 1999. Persistence of HIV-1 transcription in peripheral-blood mononuclear cells in patients receiving potent antiretroviral therapy. N Engl J Med 340:1614-1622.
26. Ganusov, V. V., N. Goonetilleke, M. K. Liu, G. Ferrari, G. M. Shaw, A. J. McMichael, P. Borrow, B. T. Korber, and A. S. Perelson. 2011. Fitness costs and diversity of the cytotoxic T lymphocyte (CTL) response determine the rate of CTL escape during acute and chronic phases of HIV infection. Journal of virology 85:10518-10528.
27. Giachetti, C., and J. J. Holland. 1988. Altered replicase specificity is responsible for resistance to defective interfering particle interference of an Sdi-mutant of vesicular stomatitis virus. J Virol 62:3614-3621.
28. Goudsmit, J., A. De Ronde, D. D. Ho, and A. S. Perelson. 1996. Human immunodeficiency virus fitness in vivo: calculations based on a single zidovudine resistance mutation at codon 215 of reverse transcriptase. J Virol 70:5662-5664.
29. Guedj, J., and A. U. Neumann. 2010. Understanding hepatitis C viral dynamics with direct-acting antiviral agents due to the interplay between intracellular replication and cellular infection dynamics. J Theor Biol 267:330-340.
30. Haseltine, E. L., J. Yin, and J. B. Rawlings. 2008. Implications of decoupling the intracellular and extracellular levels in multi-level models of virus growth. Biotechnol Bioeng 101:811-820.
31. Hinkley, T., J. Martins, C. Chappey, M. Haddad, E. Stawiski, J. M. Whitcomb, C. J. Petropoulos, and S. Bonhoeffer. 2011. A systems analysis of mutational effects in HIV-1 protease and reverse transcriptase. Nature genetics 43:487-489.
32. Holland, J. 1990. Generation and replication of defective viral genomes, p. 77-99. In F. B N and K. D M (ed.), Virology. Raven Press, New York.
33. Holland, J. J., and L. P. Villarreal. 1975. Purification of defective interfering T particles of vesicular stomatitis and rabies viruses generated in vivo in brains of newborn mice. Virology 67:438-449.
34. Horodyski, F. M., and J. J. Holland. 1980. Viruses isolated from cells persistently infected with vesicular stomatitis virus show altered interactions with defective interfering particles. J Virol 36:627-631.
35. Huang, A. S., and D. Baltimore. 1970. Defective viral particles and viral disease processes. Nature 226:325-327.
36. Josefsson, L., M. S. King, B. Makitalo, J. Brannstrom, W. Shao, F. Maldarelli, M. F. Kearney, W. S. Hu, J. Chen, H. Gaines, J. W. Mellors, J. Albert, J. M. Coffin, and S. E. Palmer. 2011. Majority of CD4+ T cells from peripheral blood of HIV-1-infected individuals contain only one HIV DNA molecule. Proc Natl Acad Sci USA 108:11199-11204.
37. Ke, R., and J. O. Lloyd-Smith. 2012. Evolutionary analysis of human immunodeficiency virus type 1 therapies based on conditionally replicating vectors. PLoS Comput Biol 8:e1002744.
38. Kearney, M., F. Maldarelli, W. Shao, J. B. Margolick, E. S. Daar, J. W. Mellors, V. Rao, J. M. Coffin, and S. Palmer. 2009. Human immunodeficiency virus type 1 population genetics and adaptation in newly infected individuals. J Virol 83:2715-2727.
39. Kirkwood, T. B., and C. R. Bangham. 1994. Cycles, chaos, and evolution in virus cultures: a model of defective interfering particles. Proc Natl Acad Sci USA 91:8685-8689.
40. Klimatcheva, E., V. Planelles, S. L. Day, F. Fulreader, M. J. Renda, and J. Rosenblatt. 2001. Defective lentiviral vectors are efficiently trafficked by HIV-1 and inhibit its replication. Mol Ther 3:928-939.
41. Koldej, R. M., and D. S. Anson. 2009. Refinement of lentiviral vector for improved RNA processing and reduced rates of self inactivation repair. BMC Biotechnol 9:86.
42. Krakauer, D. C., and N. L. Komarova. 2003. Levels of selection in positive-strand virus dynamics. J Evol Biol 16:64-73.
43. Kubo, Y., K. Kakimi, K. Higo, H. Kobayashi, T. Ono, Y. Iwama, K. Kuribayashi, H. Hiai, A. Adachi, and A. Ishimoto. 1996. Possible origin of murine AIDS (MAIDS) virus: conversion of an endogenous retroviral p12gag sequence to a MAIDS-inducing sequence by frameshift mutations. J Virol 70:6405-6409.
44. Kuroda, M. J., J. E. Schmitz, W. A. Charini, C. E. Nickerson, M. A. Lifton, C. I. Lord, M. A. Forman, and N. L. Letvin. 1999. Emergence of CTL coincides with clearance of virus during primary simian immunodeficiency virus infection in rhesus monkeys. J Immunol 162:5127-5133.
45. Leigh-Brown, A. J. 1997. Analysis of HIV-1 env gene sequences reveals evidence for a low effective number in the viral population. Proc. Natl. Acad. Sci. U.S.A. 94:1862-1865.
46. Letvin, N. L., J. R. Mascola, Y. Sun, D. A. Gorgone, A. P. Buzby, L. Xu, Z. Y. Yang, B. Chakrabarti, S. S. Rao, J. E. Schmitz, D. C. Montefiori, B. R. Barker, F. L. Bookstein, and G. J. Nabel. 2006. Preserved CD4+ central memory T cells and survival in vaccinated SIV-challenged monkeys. Science (New York, N.Y 312:1530-1533.
47. Levine, B. L., L. M. Humeau, J. Boyer, R. R. MacGregor, T. Rebello, X. Lu, G. K. Binder, V. Slepushkin, F. Lemiale, J. R. Mascola, F. D. Bushman, B. Dropulic, and C. H. June. 2006. Gene transfer in humans using a conditionally replicating lentiviral vector. Proc Natl Acad Sci USA 103:17372-17377.
48. Li, D., W. B. Lott, K. Lowry, A. Jones, H. M. Thu, and J. Aaskov. 2011. Defective interfering viral particles in acute dengue infections. PLoS One 6:e19447.
49. Li, Y., J. C. Kappes, J. A. Conway, R. W. Price, G. M. Shaw, and B. H. Hahn. 1991. Molecular characterization of human immunodeficiency virus type 1 cloned directly from uncultured human brain tissue: identification of replication-competent and -defective viral genomes. J Virol 65:3973-3985.
50. Markowitz, M., M. Louie, A. Hurley, E. Sun, M. Di Mascio, A. S. Perelson, and D. D. Ho. 2003. A novel antiviral intervention results in more accurate assessment of human immunodeficiency virus type 1 replication dynamics and T-cell decay in vivo. J Virol 77:5037-5038.
51. Marriott, A. C., and N. J. Dimmock. 2010. Defective interfering viruses and their potential as antiviral agents. Rev Med Virol 20:51-62.
52. McLain, L., S. J. Armstrong, and N. J. Dimmock. 1988. One defective interfering particle per cell prevents influenza virus-mediated cytopathology: an efficient assay system. J Gen Virol 69 (Pt 6):1415-1419.
53. Metzger, V. T., J. O. Lloyd-Smith, and L. S. Weinberger. 2011. Autonomous targeting of infectious superspreaders using engineered transmissible therapies. PLoS Comput Biol 7:e1002015.
54. Michel, N., I. Allespach, S. Venzke, 0. T. Fackler, and O. T. Keppler. 2005. The Nef protein of human immunodeficiency virus establishes superinfection immunity by a dual strategy to downregulate cell-surface CCR5 and CD4. Curr Biol 15:714-723.
55. Moore, M. D., W. Fu, 0. Nikolaitchik, J. Chen, R. G. Ptak, and W. S. Hu. 2007. Dimer initiation signal of human immunodeficiency virus type 1: its role in partner selection during RNA copackaging and its effects on recombination. J Virol 81:4002-4011.
56. Mukherjee, R., G. Plesa, S. Sherrill-Mix, M. W. Richardson, J. L. Riley, and F. D. Bushman. 2010. HIV sequence variation associated with env antisense adoptive T-cell therapy in the hNSG mouse model. Mol Ther 18:803-811.
57. Neher, R. A., and T. Leitner. 2010. Recombination rate and selection strength in HIV intra-patient evolution. PLoS computational biology 6:e1000660.
58. Nelson, G. W., and A. S. Perelson. 1995. Modeling defective interfering virus therapy for AIDS: conditions for DIV survival. Mathematical Biosciences 125:127-153.
59. Nowak, M., and R. May. 2000. Virus dynamics: mathematical principles of immunology and virology. Oxford University Press, Oxford; New York.
60. Nowak, M. A., A. L. Lloyd, G. M. Vasquez, T. A. Wiltrout, L. M. Wahl, N. Bischofberger, J. Williams, A. Kinter, A. S. Fauci, V. M. Hirsch, and J. D. Lifson. 1997. Viral dynamics of primary viremia and antiretroviral therapy in simian immunodeficiency virus infection. J. Virol. 71:7518-7525.
61. O'Hara, P. J., F. M. Horodyski, S. T. Nichol, and J. J. Holland. 1984. Vesicular stomatitis virus mutants resistant to defective-interfering particles accumulate stable 5′-terminal and fewer 3′-terminal mutations in a stepwise manner. J Virol 49:793-798.
62. Paik, S. Y., A. Banerjea, C. J. Chen, Z. Ye, G. G. Harmison, and M. Schubert. 1997. Defective HIV-1 provirus encoding a multitarget-ribozyme inhibits accumulation of spliced and unspliced HIV-1 mRNAs, reduces infectivity of viral progeny, and protects the cells from pathogenesis. Hum Gene Ther 8:1115-1124.
63. Paun, A., K. Shaw, S. Fisher, L. M. Sammels, M. W. Watson, and M. W. Beilharz. 2005. Quantitation of defective and ecotropic viruses during LP-BM5 infection by real time PCR and RT-PCR. J Virol Methods 124:57-63.
64. Perelson, A. S., S. Bonhoeffer, H. Mohri, and D. D. Ho. 1999. T cell turnover in SIV infection. Science 284:555c.
65. Perelson, A. S., A. U. Neumann, M. Markowitz, J. M. Leonard, and D. D. Ho. 1996. HIV-1 dynamics in vivo: virion clearance rate, infected cell life span, and viral generation time. Science 271:1582-1586.
66. Perrault, J., and J. J. Holland. 1972. Absence of transcriptase activity or transcription-inhibiting ability in defective interfering particles of vesicular stomatitis virus. Virology 50:150-170.
67. Rouzine, I. M., and J. M. Coffin. 1999. Linkage disequilibrium test implies a large effective population number for HIV in vivo. Proc. Natl. Acad. Sci. U.S.A 96:10758-10763.
68. Rouzine, I. M., and J. M. Coffin. 1999. Search for the mechanism of genetic variation in the pro gene of human immunodeficiency virus. J. Virol. 73:8167-8178.
69. Rouzine, I. M., A. Rodrigo, and J. M. Coffin. 2001. Transition between stochastic evolution and deterministic evolution in the presence of selection: general theory and application to virology [review]. Microbiol. Mol. Biol. Rev. 65:151-185.
70. Rouzine, I. M., R. A. Sergeev, and A. I. Glushtsov. 2006. Two types of cytotoxic lymphocyte regulation explain kinetics of immune response to human immunodefiency virus. Proc Natl Acad Sci USA 103:666-671.
71. Rouzine, I. M., and L. Weinberger. 2012. The quantitative theory of within-host viral evolution. J. Stat. Mech.: Theory and Experiment.
72. Sergeev, R. A., R. E. Batorsky, and I. M. Rouzine. 2010. Model with two types of CTL regulation and experiments on CTL dynamics. J Theor Biol 263:369-384.
73. Steinhauer, D. A., J. C. de la Torre, E. Meier, and J. J. Holland. 1989. Extreme heterogeneity in populations of vesicular stomatitis virus. J Virol 63:2072-2080.
74. TBBG. 2011 Los Alamos HIV Sequence Database.
75. Temin. 1997. Presented at the Retroviruses, Cold Spring Harbor (N.Y.).
76. Thompson, K. A., and J. Yin. 2010. Population dynamics of an RNA virus and its defective interfering particles in passage cultures. Virol J 7:257.
77. Turner, A. M., J. De La Cruz, and K. V. Morris. 2009. Mobilization-competent Lentiviral Vector-mediated Sustained Transcriptional Modulation of HIV-1 Expression. Mol Ther 17:360-368.
78. Voynow, S. L., and J. M. Coffin. 1985. Truncated gag-related proteins are produced by large deletion mutants of Rous sarcoma virus and form virus particles. J. Virol. 55:79-85.
79. Wang, D., C. B. Hicks, N. D. Goswami, E. Tafoya, R. M. Ribeiro, F. Cai, A. S. Perelson, and F. Gao. 2011. Evolution of drug-resistant viral populations during interruption of antiretroviral therapy. Journal of virology 85:6403-6415.
80. Weinberger, L. S., D. V. Schaffer, and A. P. Arkin. 2003. Theoretical design of a gene therapy to prevent AIDS but not human immunodeficiency virus type 1 infection. J Virol 77:10028-10036.

### Supplemental Information (Including Methods)

Introductory Note on Terminology for Models

Throughout this supplementary information section (and the main text) we refer to two scales of models: (i) the intracellular model which is at the scale of a single infected cell, and (ii) the in vivo model which refers to the scale of the individual infected person or “host”. Though “in vivo” can refer to cells in a tissue-culture setting (especially in the field of biochemistry), in an attempt to minimize confusion we follow the usage in the virology and HIV fields which use in vivo to refer exclusively to the level of the whole organism (i.e. within patients or non-human primates).

A. Capsid Stealing Model

Basic Equations and Biological Interpretation

Consider a cell with an integrated HIV provirus. Of all virus products, we focus on two: C(t), the amount of fully formed capsids that do not yet carry genomic mRNA dimers, and G(t), the amount of dimers of genomic mRNA. The system of equations has the form

$$\begin{matrix}\frac{d G}{d t} = \underbrace{θ}_{\begin{matrix}HIV genome \\ production\end{matrix}} - \underbrace{{k}_{pck} GC}_{\begin{matrix}Packaging of \\ HIV genomes \\ into capsids\end{matrix}} - {\underbrace{α G}}_{\begin{matrix}Loss of \\ HIV genomes\end{matrix}} & ( 1 ) \\ \frac{d C}{d t} = \underbrace{ηθ}_{\begin{matrix}Capsid \\ production\end{matrix}} - {k}_{pck} GC - \underbrace{β C}_{\begin{matrix}Loss of \\ capsids\end{matrix}} & ( 2 )\end{matrix}$$

Model parameters are: θ, the linear production rate of HIV genomes; k_{pck}, packaging efficiency; α and β, the exponential rates of genome and capsid loss, respectively; and, η, the capsid-to-genome production ratio.

In a cell infected with HIV provirus and co-infected with m copies of a (DIP) provirus, the system of equations (as given in the main text in Eqs. 1-3) has the form:

$$\begin{matrix}\frac{d G}{d t} = \underbrace{θ}_{\begin{matrix}HIV genome \\ production\end{matrix}} - \underbrace{{k}_{pck} GC}_{\begin{matrix}Packaging of \\ HIV genomes \\ into capsids\end{matrix}} - {\underbrace{α G}}_{\begin{matrix}Loss of \\ HIV genomes\end{matrix}} & ( 3 ) \\ \frac{d C}{d t} = \underbrace{ηθ}_{\begin{matrix}Capsid \\ production\end{matrix}} - {k}_{pck} ( G + {G}_{DIP} ) C - \underbrace{β C}_{\begin{matrix}Loss of \\ capsids\end{matrix}} & ( 4 ) \\ \frac{d {G}_{DIP}}{d t} = \underbrace{mP θ}_{\begin{matrix}DIP genome \\ production\end{matrix}} - \underbrace{{k}_{pck} {G}_{DIP} C}_{\begin{matrix}Packaging of \\ DIP genomes \\ into capsids\end{matrix}} - \underbrace{α {G}_{DIP}}_{\begin{matrix}Loss of \\ DIP genomes\end{matrix}} & ( 5 )\end{matrix}$$

where G_{DIP}(t) is concentration of DIP genomes, and parameter P is the ratio of DIP to HIV genome production rates. As previously demonstrated (1, 2), P>1 is required for a therapeutic effect so P>1 is used. Multiplicity of DIP infection m is any integer number, m=1, 2, 3, . . . .

The packaging coefficients for HIV and DIP are assumed to be the same (k_{pck}). Indeed, mutation in the packaging domain of HIV gag equally affects packaging of particles that share the same stem loop 3 (SL3) sequence. Below, we study evolution of HIV affecting k_{pck}. (A double mutation in HIV, one in gag decreasing k_{pck} and another in HIV-1 stem-loop 3—SL3, the so-called Ψ region—compensating for this effect, could decrease k_{pck} for DIP but not for HIV. However, the same compensatory mutation will occur in DIP SL3, only much more quickly, because it is a single mutation. Therefore equality of the two packaging constants will be preserved.)

Below we will consider a dually infected cell (containing an integrated HIV provirus and m copies of integrated DIP provirus) because a singly infected cell can be considered a particular case of a dually infected cell with P=0 or m=0.

Stead-State Calculations

Here, we assume that sufficient time has passed after infection of a cell so that steady state has been reached. For dually infected cells (Eqs. 3-5), steady-state amounts of genomes and capsids are given by the equations:

$$\begin{matrix}G = \frac{θ}{α + {k}_{pck} C} & ( 6 ) \\ C = \frac{ηθ}{β + {k}_{pck} ( G + {G}_{DIP} )} & ( 7 ) \\ {G}_{DIP} = \frac{mP θ}{α + {k}_{pck} C} & ( 8 )\end{matrix}$$

It is convenient to introduce a rescaled capsid number y defined as

y=k_{pck}C/α (9)

In this notation, Eqs. 6-8 are equivalent to

$$\begin{matrix}G = \frac{θ}{α ( 1 + y )} & ( 10 ) \\ {G}_{DIP} = \frac{mP θ}{α ( 1 + y )} \\ where & ( 11 ) \\ κ {y}^{2} + ( mP + 1 - η + κ ) y - η = 0 \\ and & ( 12 ) \\ κ = \frac{αβ}{θ {k}_{pck}} & ( 13 )\end{matrix}$$

Here, κ is the composite “waste parameter” contrasting the loss of HIV genomes and capsids against genome production and packaging. The solution of Eq. 12 has the form

$$y = \frac{1}{2 κ} ⌊ - ( mP + 1 - η + κ ) + \sqrt{{( mP + 1 - η + κ )}^{2} + 4 ηκ} ⌋ \qquad ( 14 )$$

Burst Sizes of HIV and DIP and Connection to the In Vivo Level

To connect to HIV and DIP dynamics at the level of an individual host, we need to predict the burst size (total number of particles produced per cell lifetime) of HIV in singly (HIV+DIP−) and dually (HIV+DIP+) infected cells, and that of DIP in dually infected cells. Based on previous analysis (1), we assume that steady-state viral production is reached shortly after the cell is infected and long before the death of the infected cell. Then, the total numbers of virus particles per cell are given by

n=k_{pck}[GC]_{P=0}/δ (15)

ψₘn=k_{pck}GC/δ (16)

ρₘψₘn=k_{pck}G_{DIP}C/δ (17)

Here, n is the HIV burst size from a cell infected with HIV only (the case obtained by setting P=0), ψₘ shows decrease in HIV burst size due to co-infection with DIP, ρₘ is the ratio of DIP to HIV burst size in a co-infected cell, and 1/δ is the average lifetime of an HIV-infected cell.

Substituting Eqs. 9-11 for steady-state values of G, G_{DIP}, and C into Eqs. 15-17, we arrive at

$$\begin{matrix}n = \frac{θ}{δ} \frac{y}{1 + y} {\vert}_{P = 0} & ( 18 ) \\ {ψ}_{m} n = \frac{θ}{δ} \frac{y}{1 + y} & ( 19 ) \\ {ρ}_{m} = \frac{{G}_{DIP}}{G} = mP & ( 20 )\end{matrix}$$

Here, the rescaled capsid concentration, y, is given by Eq. 14 and the multiplicity of infection, m, runs from 1 to infinity.

Case of Small Waste Parameter κ<<1

As we show in Section C below, HIV evolution is directed towards decrease of the waste parameter. Therefore, the case of small κ is of considerable practical interest. When κ<<1, Eq. 14 for y can be approximated by a simpler expression depending on sign of mP+1−η, as given by

$$y = ⌊ \begin{matrix}\frac{η}{1 + mP - η} & η < mP + 1 \\ \frac{η - 1 - mP}{κ} & η > mP + 1 ( 22 )\end{matrix} \qquad ( 21 )$$

Substituting y from Eqs. 21 and 22 into Eqs. 18 and 19, and evaluating them in the limit κ→0, we obtain

$$\begin{matrix}n = ⌊ \begin{matrix}θ / δ , & η > 1 \\ ηθ / δ & η < 1\end{matrix} \\ and & ( 23 ) \\ {ψ}_{m} n = ⌊ \begin{matrix}\frac{θ}{δ} & η > 1 + mP \\ \frac{θ}{δ} \frac{η}{1 + mP} & η < 1 + mP\end{matrix} & ( 24 )\end{matrix}$$

respectively. Combining Eqs. 23 and 24, for the value of the HIV suppression factor in dually infected cells yₘ we obtain

$${ψ}_{m} = ⌊ \begin{matrix}\frac{1}{1 + mP} & η < 1 \\ \frac{η}{1 + mP} & 1 < η < 1 + mP ( 26 ) \\ 1 & η > 1 + mP ( 27 )\end{matrix} \qquad ( 25 )$$

B. HIV and DIP Load at the Level of an Individual Host

Basic Equations and Biological Interpretation

We begin with the well-parameterized “standard” model of HIV-virus in vivo dynamics (3, 4) and, similar to the method we have previously used (1, 2), we generalize this model to include production of DIP particles. The generalized model includes co-infection of cells with DIP and HIV, so that dually infected cells produce less HIV. In comparison to the previous versions (1, 2), we relax the restriction of one copy of DIP provirus per cell. Based on results of recent in vivo studies (5), we postulate a single HIV provirus per cell.

The system of equations has the form:

$$\begin{matrix}\frac{d T}{d t} = b - ( d + kV + {kV}_{DIP} ) T & ( 28 ) \\ \frac{d I}{d t} = kVT - δ I & ( 29 ) \\ \frac{d {T}_{DIP m}}{d t} = {kV}_{DIP} {T}_{IP m - 1} - ( d + kV + {kV}_{DIP} ) {T}_{DIP m} , \\ m = 1 , 2 , 3 , … & ( 30 ) \\ \frac{d {I}_{D m}}{d t} = {kVT}_{DIP m} - δ {I}_{D m} , m = 1 , 2 , 3 , … & ( 31 ) \\ \frac{d V}{d t} = n δ I + n δ {\sum }_{m = 1}^{∞} {ψ}_{m} {I}_{D m} - cV & ( 32 ) \\ \frac{d {V}_{DIP}}{d t} = n δ {\sum }_{m = 1}^{∞} {ρ}_{m} {ψ}_{m} {I}_{D m} - {cV}_{DIP} & ( 33 )\end{matrix}$$

Here, the state variables are: T, uninfected CD4 T cells permissive for viral replication; I, cells infected with HIV only; T_{DIP m}, CD4 T cells harboring m copies of DIP provirus but not infected with HIV (by definition, T_{DIP 0}=7); I_{D m}, “dually infected” cells harboring a copy of HIV and m copies of DIP provirus; V, HIV load (free virus concentration in peripheral blood plasma); V_{DIP}, DIP load.

The model parameters, which are well described in the literature, are: b, linear production rate of uninfected cells; d, natural death rate of uninfected cells; k, infectivity factor; 6, death rate of singly and dually infected cells; n, HIV burst size from a singly infected cell. There are two additional parameters in the presence of DIP: n_{ψm}, HIV burst size from a dually infected cell with m copies of DIP provirus; and nρ_{mψm}, DIP burst size from a dually infected cell with m copies of DIP provirus.

The biological interpretation of Eqs. 28-33 is that uninfected cells that are permissive for viral replication (7) are replenished from a constant source and depleted by three competing processes: (i) their natural death, (ii) infection by HIV particles, (iii) or infection by IPs (Eq. 28). Cells that become infected by HIV (I) produce viral particles and die at average rate δ˜1/day (Eq. 29). Alternatively, before becoming infected with HIV, a cell can be infected with one or more copies of DIP provirus (T_{IP}) and we classify these cells according to the copy number of DIP proviruses by cell ‘bins’ T_{IP 1}, T_{IP 2}, T_{IP 3}, . . . , T_{IP m}, . . . (Eq. 30). Cells infected with DIP alone do not express HIV proteins and die at the same rate as uninfected cells. If a T_{IP} cell is subsequently infected with HIV, the cell becomes “dually infected” (I_{D m},) and begins producing both HIV and DIP particles (Eq. 31). These dually infected cells (I_{D m}) are HIV⁺DIP⁺ and die as rapidly as singly infected cells, I, which are HIV⁺DIP⁻. Thus, HIV particles are generated from both singly and dually HIV-infected cells (Eq. 32).

Steady-State Calculations

Chronic HIV infection represents an approximate steady state. Setting the right-hand side (RHS) of Eqs. 28-33 to zero, we obtain

$$\begin{matrix}T = \frac{b}{d ( 1 + v + {v}_{DIP} )} & ( 34 ) \\ I = \frac{kVT}{δ} = \frac{bv}{d ( 1 + v + {v}_{DIP} )} & ( 35 ) \\ {T}_{DIP m} = {Tq}^{m} & ( 36 ) \\ {I}_{D m} = {Iq}^{m} & ( 37 ) \\ 1 + v + {v}_{DIP} = {R}_{0} ( 1 + {\sum }_{m = 1}^{∞} {ψ}_{m} {q}^{m} ) or v = 0 & ( 38 ) \\ {( 1 + v + {v}_{DIP} )}^{2} = {R}_{0} v {\sum }_{m = 1}^{∞} {ρ}_{m} {ψ}_{m} {q}^{m - 1} or {v}_{DIP} = 0 & ( 39 )\end{matrix}$$

where, for tractability, the following new notation is used:

$$\begin{matrix}{R}_{0} = \frac{nkb}{cd} & ( 40 ) \\ v = \frac{kV}{d} , {v}_{DIP} = \frac{{kV}_{DIP}}{d} & ( 41 ) \\ q = \frac{{V}_{DIP}}{d + kV + {kV}_{DIP}} = \frac{{v}_{DIP}}{1 + v + {v}_{DIP}} & ( 42 )\end{matrix}$$

Here, R₀ is the basic reproduction ratio in the beginning of infection, v and v_{DIP} are rescaled HIV and DIP loads. New notation q determines the average number of integrated DIP provirus copies E[m] in a dually infected cell, as given by

$$E [ m ] = \frac{{\sum }_{m = 1}^{∞} {mq}^{m}}{{\sum }_{m = 1}^{∞} {q}^{m}} = \frac{q \frac{d}{d q} {\sum }_{m = 1}^{∞} {q}^{m}}{{\sum }_{m = 1}^{∞} {q}^{m}} = \frac{1}{1 - q} \qquad ( 43 )$$

HIV load (v) and DIP load (V_{DIP}) in Eqs. 34-39 can be obtained by solving Eqs. 38 and 39 together with respect to v and v_{DIP}. Note that q entering these equations depends on v and v_{DIP} as given by Eq. 42 and must be calculated to be self-consistent.

Calculation for FIG. 2: HIV Load is Stably Decreased by the Presence of DIP

MATLAB™ (version R2011a) was used to perform the calculation of q, v and v_{DIP} through numerical iteration (although in certain important cases, such as the case of small κ and large P, this calculation can be performed analytically, with asymptotic accuracy). The two parameters of the in vivo model reflecting the effect of DIP, ρₘ and ψₘ, can be expressed in terms of intracellular parameters κ, η, and mP, as given by Eqs. 14 and 18-20. Therefore, the total rescaled HIV load and the total DIP load, as well as other important properties of the steady state in an individual, depend on four dimensionless parameters: R₀, P, κ, and η. Results for HIV and DIP loads as functions of κ and P at different η are shown in FIG. 2.

We observe that HIV is stably suppressed by DIP in a broad parameter range (FIGS. 2B and D). One reason is that multiple infection of cells by DIP amplifies its effect on HIV. The average multiplicity of DIP infection, E[m]=1/(1-q), is rather large even at modest values of η and P (see FIG. 7a, below). Indeed, restricting DIP MOI to one, as previously assumed in ref. (1), considerably limits the degree and the parameter range of DIP interference (see FIG. 7b). In agreement with previous findings (1), the degree of HIV suppression and DIP load are rather sensitive to the DIP-to-HIV expression asymmetry, P (FIG. 2D-E).

The decrease in HIV load, as compared to its value at κ=0, R₀=10, is only partly due to the presence of DIP. The remainder of the decrease is due to increased waste, κ, which decreases the HIV burst size n (Eqs. 14 and 18). We factored in this increased-waste effect by changing the value of R₀ proportionally. For reference, HIV load at zero DIP load (i.e., at P=0) is shown in FIG. 2B as black lines. The contribution of DIP to the decrease of HIV load at each given κ is also shown (FIG. 6).

Dynamic Stability of DIP In Vivo

In principle, it is important to determine the parameter range in which the HIV-1 steady state with DIP is stable. However, because we are ultimately interested in analyzing whether DIP can autonomously spread between HIV-infected individuals as in (1), we use a more stringent criterion: we analyze whether DIP-free states are unstable (i.e., whether a small amount of DIP added to an DIP-free steady-state virus population will expand and result in a new steady state, where both HIV and DIP are present). We start from the DIP-free state (V_{DIP}=I_{Dm}=T_{DIPm}=0) and Eqs. 34-39 reduce to a well-known result (for reviews, see refs. (3, 4))

$$T = \frac{b}{{dR}_{0}} \\ V = \frac{d}{k} ( {R}_{0} - 1 ) \\ I = \frac{b ( {R}_{0} - 1 )}{{dR}_{0}} \qquad ( 44 )$$

At time t=0, we introduce a small amount of DIP, V_{DIP}(0), and determine whether V_{DIP}(t), I_{Dm}(t), and T_{DIPm}(t) will expand or contract in time. We do not need to solve dynamics of the entire set of Eqs. 28-33 because DIP load is initially low and DIP-infected cells are initially few such that HIV-related variables T, V, and I are weakly perturbed and can be approximated with their previous respective steady-state levels. Hence, V_{DIP}(t), I_{Dm}(t), and T_{DIP m}(t) obey linearized versions of Eqs. 30, 31, and 33 given by

$$\frac{d {T}_{DIP 1}}{d t} = {kT}^{ss} {V}_{DIP} - ( d + {kV}^{ss} ) {T}_{DIP 1} \\ \frac{d {I}_{D 1}}{d t} = {kV}^{ss} {T}_{DIP 1} - δ {I}_{D 1} \\ \frac{d {V}_{DIP}}{d t} = n {ρ}_{1} {ψ}_{1} {I}_{D 1} - {cV}_{DIP} \qquad ( 45 )$$

Multiply-infected cells (i.e. I_{D m} and T_{DIP m} for m>2) do not emerge here, because they correspond to 2^{nd} or higher-order terms in the small variable V_{DIP}.

At large times, the three variables in Eq. 45 depend on time as exp(λₘₐₓt), where λₘₐₓ is the largest eigenvalue of the dynamic matrix in the right-hand side, D. The condition of DIP expansion is λₘₐₓ>0. Using the standard eigenvalue equation det[D-λ1]=0, together with V^{ss} and T^{ss} from Eqs. 44, and R₀=bkn/cd, we obtain from standard eigenvalue analysis

$${ρ}_{1} {ψ}_{1} > \frac{{R}_{0}}{{R}_{0} - 1} \qquad ( 46 )$$

The equivalent condition was previously obtained for the model version that assumed a single copy of DIP provirus in dually infected cells (1). This coincidence is expected, because multiple infection with DIP is negligible when DIP load is still low.

To test for dynamic stability of DIP, steady-state HIV and DIP loads in FIG. 2C-D are plotted only in the interval of κ where the condition in Eq. 46 is met. Importantly, the curves in FIG. 2C-D end where the DIP viral load vanishes. The results are consistent with a continuous transition (in DIP load) from a stable DIP-free state to a stable steady state with DIP.

Dynamic Stability of DIP at Small Waste Parameter κ<<1

At small waste parameter, κ<<1, using Eqs. 20 and 25-26, we predict that the state with DIP is stable if

$$η > \frac{1 + P}{P} \frac{{R}_{0}}{{R}_{0} - 1} \qquad ( 47 )$$

For example, for P=5 and R₀=10, the stability interval is η>1.3. The biological meaning of condition in Eq. 47 is that for DIP to be stable, HIV must generate extra capsids for DIP to parasitize. For moderately wasteful process (κ>1), the condition is relaxed, and η can be a bit smaller than unity (see FIG. 2C, η=1 curve).

C. Evolutionary Stability of DIP

Selection Coefficient of HIV in the Presence of DIP at In Vivo Scale

The here aim is to determine whether HIV can escape its parasite and reach the region where the population of dually infected cells becomes unstable and DIP becomes extinct. To do so, we must determine the direction of HIV evolution in the presence of DIP in parameter space. In this subsection, we focus on evolution at in vivo scale (i.e. individual-patient level) and use a standard approach from population genetics based on the selection coefficient and fitness. In the next subsection, we will connect fitness to the level of intracellular dynamics using the capsid-stealing model.

The fitness of a virus strain is determined by the average progeny number, i.e., the number of cells in a new generation infected by virions from a cell from the previous generation. At steady state, the average progeny number is equal to one. If an HIV mutation occurs, the mutant strain will have a smaller or larger average progeny number; the relative difference is referred to as the “selection coefficient” s_{eff}. Depending on the sign of s_{eff}, the mutant will either expand as exp[(s_{eff}δ)t] and spread onto entire population, or go extinct. Here, 1/δ is the time interval of one generation, equal to the average lifetime of an infected cell.

Note that even a beneficial mutation emerging within a genetically diverse population is likely to become extinct due to the combination of random drift and linkage effects. Indeed, a mutation must occur within a high-fitness strain to become amplified and ‘fixed’ in a population. Complex mathematical theories have recently been developed to describe the fixation probability and the speed of evolution in asexual models and models with rare recombination (6-14). In the present work, we do not consider these complexities: our interest is in the general direction of evolution rather than its exact speed, and in the sign of s_{eff} as the pointer. We assume a deterministically large population, and a single mutation with small fitness effect, as given by |s_{eff}|<<1.

Even this relatively simple task faces obstacles. Our system comprises virus-infected cell types I(t) and I_{Dm}(t),m=1,2, with different burst sizes and different contributions to the effective selection coefficient. Our general approach to calculation of s_{eff} is as follows. We start from a steady-state population, with state variables given by Eqs. 34-39. Consider a mutation in the dominant HIV strain. We note that infectivity parameter k always enters any results as a product kn. Therefore, without any loss in generality, we will assume that only n changes due to mutations. We postulate that mutation changes all burst sizes for HIV in two cell types and for IP, as given by

n→n(1+Δₙ),ψₘ→ψₘ(1+Δψₘ),ρₘ→ρₘ(1+Δρₘ), (48)

Here increments Δₙ, Δ_{ψm}, Δ_{ρm} are considered input parameters. We then inject a small amount of the mutant virus strain, V^{mut}(0). Dynamics of the mutant subpopulations V^{mut}(t), I^{mut}(t), I_{D m}^{mut}(t) can be calculated from Eqs. 29, 31, and 32, as follows. While the mutant strain is still a small fraction of a population, it weakly perturbs the rest of population, which remains near steady state (Eqs. 34-39). We introduce rescaled sizes of mutant subpopulations, x, yₘ, and z, defined as

I^{mut}(t)=I^{ss}x(t)

I_{D m}^{mut}(t)=I_{D m}^{ss}yₘ(t)

V^{mut}(t)=V^{ss}z(t) (49)

In this notation, Eqs. 29, 31, and 32 take the form

$$\begin{matrix}\frac{d x}{d t} = ( z - x ) δ \\ \frac{d {y}_{m}}{d t} = ( z - {y}_{m} ) δ , m = 1 , 2 & ( 50 ) \\ z + \frac{1}{c} \frac{d z}{d t} = \frac{{R}_{0} ( 1 + {Δ}_{n} )}{1 + v + {v}_{DIP}} [ x + {\sum }_{i = 1}^{∞} {ψ}_{i} ( 1 + {Δ}_{ψ i} ) {q}^{i} {y}_{i} ] & ( 51 )\end{matrix}$$

(Note that the mutational change in the DIP burst size, Δ_{ρm}, does not enter these equations.) Neglecting the time derivative on the left hand side of Eq. 51 due to the strong numerical inequality c>>d and substituting R₀/(1+v+v_{DIP}) from Eq. 38 into 51, the latter simplifies to

$$z = \frac{1 + {Δ}_{n}}{1 + {\sum }_{i = 1}^{∞} {ψ}_{i} {q}^{i}} [ x + {\sum }_{i = 1}^{∞} {ψ}_{i} ( 1 + {Δ}_{ψ i} ) {q}^{i} {y}_{i} ] \qquad ( 52 )$$

The asymptotic expressions for variables x(t), yₘ (t), and z(t) at large times t have the exponential form exp[(s_{eff}δ)t], so that Eqs. 50 reduce to

z=(1+s_{eff})x=(1+s_{eff})yₘ (53)

Substituting Eq. 53 into 52 and neglecting second-order terms in Δ, we obtain

$${s}_{eff} = {Δ}_{n} + \frac{{\sum }_{i = 1}^{∞} {ψ}_{i} {q}^{i} {Δ}_{ψ i}}{1 + {\sum }_{i = 1}^{∞} {ψ}_{i} {q}^{i}} \qquad ( 54 )$$

where ψᵢ is given by Eqs, 14, 18, 19 (for k=0, by simplified Eqs. 25 to 27), and q is found from solving Eqs. 38, 39, and 42 (result in FIG. 7a). Thus, the selection coefficient s_{eff} has contributions from two relative changes caused by mutation: in the base burst size, n, and in the HIV suppression factor due to the presence of i copies of DIP, ψᵢ.

Specific Expression for the Selection Coefficient in the Intracellular Model

In the previous subsection, we expressed the effective selection coefficient of mutation in the HIV genome, in the general form, in terms of relative changes in burst sizes n, n_{ψm}, and nρ_{mψm} due to mutation. Now, we will express it in terms of parameters of the single-cell model. The burst sizes are determined by η, κ, P, m, and θ/δ (Eqs. 14, and 18-20). The expression asymmetry, P, is fixed by the molecular architecture of the DIP and m is the index in a sum. It is also obvious that HIV evolves towards larger capsid numbers η (see two subsections down) and θ/δ, but there is a natural limit to such an increase, and it does not reflect on DIP stability since η increases all burst sizes equally (both the HIV burst from dually and singly infected cells and the DIP burst from dually infected cells). Therefore, we focus on evolution in the remaining parameter, the waste parameter κ (defined by Eq. 13). κ can evolve, for example, by changing packaging parameter k_{pck}, which is controlled by the amino-acid sequence in HIV gag and the corresponding RNA sequence in the HIV SL3 loop. Since gag and SL3 mutations would reduce DIP stealing but also reduce HIV burst size, the direction of evolution in κ is not obvious.

Calculations for FIG. 3: Effect of Mutation in the Waste Parameter κ

We denote mutational change in parameter κ as ∂κₘᵤₜ. Mutations that relax packaging result in increased κ, ∂κₘᵤₜ>0. In singly infected cells (I), such a mutation is deleterious to HIV because it decreases the burst size. However, singly infected cells are rare, and their fraction is on the order of 1/E[m]˜1−q (Eq. 37), where 1−q is small (FIG. 7a). In the dominant population of dually infected cells (I_{Dm}), the same mutation may be favorable, because reduction in packaging also reduces capsid stealing by DIP. Formally, the two terms in the numerator of Eq. 54 should have different signs. Below, we confirm this intuitive prediction and show that both effects are of the same order, but the first effect (deleterious reduction in HIV burst size) wins and results in selection against increases in κ.

From Eqs. 18-19, for the relative changes in the HIV burst size in singly and dually infected cells (n and n_{ψm}, respectively), we obtain

$${Δ}_{n} = \partial {κ}_{mut} \frac{\partial }{\partial κ} {ln [ \frac{y}{1 + y} ]}_{P = 0} \\ {Δ}_{n} + {Δ}_{ψ m} = \partial {κ}_{mut} \frac{\partial }{\partial κ} ln \frac{y}{1 + y} \qquad ( 55 )$$

respectively, where y as a function of κ is given by Eq. 14. Substituting Δₙ and Δ_{ψm} from Eqs. 55 into 54 and computing q numerically from Eqs. 38, 39, and 42, we calculate the desired value of s_{eff}.

The results are shown in FIG. 3. As expected, the factor of HIV suppression by DIP favors mutations increasing waste parameter κ. However, the overall decrease in the HIV burst size dominates evolution: HIV evolves towards smaller waste parameters. We conclude that HIV cannot shake off DIP by bringing it to the threshold of extinction.

We assumed equal packaging constants for DIP and HIV, since a mutation in HIV gag is used by both HIV and DIP and decreases the two constants equally. In principle, a second compensatory mutation in HIV SL3 loop could partly restore packaging for HIV while leaving DIP packaged inefficiently. However, an identical mutation in SL3 loop of DIP will immediately restore high packaging efficiency of DIP. Compensation in DIP will occur rapidly, because it occurs in a larger population (DIP provirus population is larger than HIV provirus population) and is a single mutation rather than a pair of corresponding mutations in Gag structure and SL3 needed for HIV to switch to an alternate efficient packaging scheme. The rate of single mutation in DIP can be estimated to be higher by a factor of V_{DIP}s_{eff}/Vμ. Thus, unlike in the case of the genome-stealing mechanism (main text), compensatory mutation in HIV does not cause divergent evolution of HIV and DIP.

Effect of Mutation in the Capsid-to-Genome Production Ratio h

In the same way, we can predict the effective selection coefficient for mutations increasing the capsid-to-genome production ratio η by ∂ηₘᵤₜ. Replacing the derivatives in κ with derivatives in η in Eq. 55, leads to the following forms for relative changes in burst sizes of HIV in singly and dually infected cells, respectively:

$${Δ}_{n} = \partial {η}_{mut} \frac{\partial }{\partial η} {ln [ \frac{y}{1 + y} ]}_{P = 0}$$

$${Δ}_{n} + {Δ}_{ψ m} = \partial {η}_{mut} \frac{\partial }{\partial η} ln \frac{y}{1 + y}$$

where y is determined by Eq. 14.

As we have shown in the previous subsection, HIV evolves toward smaller waste parameters, κ<<1. In the limit of small κ, it is more convenient to use directly Eqs. 23 and 25-27 for the burst size n and the suppression factor _{ψm}, to find

$$\begin{matrix}{Δ}_{n} = ⌊ \begin{matrix}\partial {η}_{mut} / η , & η < 1 \\ 0 , & η > 1\end{matrix} \\ and & ( 56 ) \\ {Δ}_{ψ m} = ⌊ \begin{matrix}0 , & η < 1 \\ \partial {η}_{mut} / η & 1 < η < 1 + mP \\ 0 , & η > 1 + mP\end{matrix} & ( 57 )\end{matrix}$$

The selection coefficient s_{eff} is derived by substituting the relative changes, Eqs. 56 and 57, into Eq. 54, In Eq. 54, we use Eqs. 25 to 27 for suppression factor yᵢ. and numeric solution of Eqs. 38, 39, and 42 for q.

Here we assume that DIP is dynamically stable in an individual host, which is true under the condition η>η_{c}=(P+1)R₀/[P(R₀−1)] where η_{c} is slightly larger than 1 (Eq. 47). To take DIP stability into account, the interval boundary between the first and second interval in Eq. 57 is slightly shifted from to η=1 to η=η_{c}

The final result for selection coefficient of mutation in η is shown in FIG. 4. As expected, HIV favors increase in capsid production in the entire range of η. Indeed, both terms in Eq. 54 are non-negative, and we have positive selection coefficient s_{eff}>0. The magnitude of the selection coefficient, however, depends on η and the presence of IP. At η<1, when DIP is absent, HIV strongly favors increases in η due to increases in its burst size (first term in Eq. 54; 2^{nd} term is zero). At η_{c}<η<P+1, increases in capsid production is also strongly preferred, due to the presence of DIP, which steals most capsids in dually infected cells (second term in Eq. 54; first term in Eq. 54 is zero).

Interestingly, in a narrow interval of η, such that 1<η<η_{c}, selection coefficient is zero (in Eq. 54, Δₙ=q=0). Intuitively, HIV already has more capsids than it needs to package its genomes (i.e. η>1) and DIP suppression is absent. Only a finite rate of product loss (i.e. finite k) weakly favors production of extra capsids and results in small, positive selection coefficient (in Eq. 54, q=0, Δₙ>0).

Suppose, an HIV strain with η<1 infects a person in a population infected with an older strain of HIV with DIP present. During evolution within the person, the value of η in the new strain will evolve rapidly to η=1; then, its increase will slow down significantly. When the DIP stability threshold η=η_{c} is reached, DIP can enter by co-infecting the person from an outside population. Further increases in η will accelerate again until the biological ceiling (due to mRNA degradation) is reached. This raises interesting questions regarding competition in human populations between two HIV strains with a high and a low upper limit on capsid production (the main text, Discussion).

D. Estimate of Intracellular Model Parameters κ and η for HIV In Vivo

So far, our consideration was general. It is instructive to place parameters within the context of HIV infection in vivo. In FIGS. 2-3 the results depend on four parameters R₀, P, η, and κ. R₀ is set in patients (with an average of R₀˜10) and P is set by the molecular design of the DIP, which leaves the parameters η and κ. Direct estimates of these two parameters may be difficult, because they describe the rate of processes consisting of many consecutive stages. Below, we estimate them indirectly, relating η and κ to two dynamic quantities, the successful fractions of genomes and capsids, f_{G} and f_{C}, respectively. By definition, successful genomes are those that do not decay but are packaged within released virions. “Successful” capsids are released with a dimerized HIV genome inside, rather than a single RNA copy, no RNA (i.e. empty) or with irrelevant non-gRNAs. From in vivo data in the literature, we can estimate f_{C} and obtain a relation between η and κ.

We consider cells infected with HIV only. Steady-state conditions for Eqs. 1 and 2 have the form

θ=k_{pck}GC+αG

ηθ=k_{pck}GC+βG (58)

By definition, fractions of “successful” capsids and genomes are given by

$${f}_{C} = \frac{{k}_{pck} GC}{ηθ} , {f}_{G} = \frac{{k}_{pck} GC}{θ} \qquad ( 59 )$$

which yields

η=f_{G}/f_{C} (60)

Definition of parameter κ in Eq. 13 can be written as

$$κ = \frac{α G}{θ} \frac{β C}{{k}_{pck} GC} = ( 1 - {f}_{G} ) \frac{( 1 - {f}_{C} )}{{f}_{C}} \qquad ( 61 )$$

where we used Eqs. 56-57. Excluding f_{G} from Eqs. 60 and 61, we obtain a linear relationship between η and κ

$$η + \frac{κ}{1 - {f}_{C}} = \frac{1}{{f}_{C}} \qquad ( 62 )$$

In principle, the fraction of non-empty released capsids f_{C} is measurable and has been estimated previously as f_{C}˜0.2 (Ref. (15), Appendix D). The estimate compared two measurements of the average viremia peaks measured in MAMU A*01 rhesus macaques infected with SIVmac251: one by p24 Ab assay (16) and another by sensitive branching DNA assay (17, 18). A recent in vitro study, using a two-RNA labeling technique, predicted a much higher value, f_{C}>0.9, for an engineered HIV strain infecting a cell line (19). However, due to assay fidelity, the reliability of these estimates of f_{C} in vivo must be taken with a degree of caution.

The relationship between η and κ given by Eq. 60 and the region of dynamic instability of DIP is shown in FIG. 6. Thus, the larger η the smaller κ, and both do not exceed 1/f_{C}˜5 (or larger). Because HIV tends to evolve towards small κ and large η (FIG. 3 and sections above), it is reasonable to conjecture that η is close to 1/f_{C} and far from the instability region of DIP. Studies in vitro in broader range of cell types could verify the value of f_{C} and the inferred capsid-to-genome ratio.

FIG. 5. Evolution in the dimerization initiation sequence leads to sequence divergence between HIV and DIP. An example of an evolving palindromic SL1 sequence is shown on the top. Initial sequence is the same for DIP and HIV, GCGCGC, IP sequence remains unchanged. Dimerization coefficients for HIV-HIV, HIV-DIP and DIP-DIP (defined in Equations in FIG. 1b) are shown qualitatively versus mutation pair number. Each pair includes a mutation in the 6-residue SL1 loop causing a palindrome mismatch, and a compensatory mutation, which restores palindrome. Each dimerization constant k_{H}, k_{IP}, k_{HIP}, has an idealized component determined by the number of mismatches (see FIG. 1c-d for details), and a fluctuating component, which depends on specific palindrome sequence (and SL1 stem sequence as well). HIV-DIP cross-dimerization coefficient decreases as the loop sequence diverges from GCGCGC, while HIV-HIV dimerization coefficient fluctuates around a constant level.

FIG. 6. DIP contribution to suppression of HIV-1 viral load. The curves show the ratio of HIV load to its value in the absence of DIP (P=0). Values of η and P are shown. When both P and η are sufficiently large, >5, the main decrease in HIV load at finite κ is due to suppression by DIP rather than to the loss of HIV products (compare to FIG. 2b).

FIG. 7. The average multiplicity of DIP infection is high, which enhances suppression of HIV-1. (a) Average number of DIP provirus copies per cell E[m]=1/(1−q) is shown as a function of waste parameter κ, at several values of η: η=2 (red), η=5 (green), η=10 (blue). Values in the vertical axis are calculated as 1/(1−q), where q is the ratio of the cell number with m+1 copies to the cell number with m copies, (see Methods in SI Text). Unity at the vertical axis corresponds to a DIP-free population. The DIP subpopulation is unstable at small and large κ when η˜1, and at large κ when η>1.7. Multiple copies of DIP provirus per cell amplify DIP genomic mRNA amount in cells and at the level of the individual, and amplify suppression of HIV-1 load. (b) Steady-state HIV-1 load when multiplicity of DIP infection, m, is restricted to ≦1. HIV-1 suppression is markedly decreased at larger η (cf. FIG. 2A) since a single copy of DIP provides much weaker interference; at m≦1 suppression of HIV-1 is primarily due to the loss of HIV-1 products at large κ (cf. FIG. 2b). Parameters used are as described in main text FIG. 2 (i.e. R₀=10, P=5).

FIG. 8. In vivo estimates for waste parameter κ and capsid-to-genome production η are inversely related. The fraction of non-empty virions is f_{C}˜0.2 for HIV, according to (15, 20). The likely region of actual parameters (thick red line) is far from regions of DIP instability (orange shade, compare with FIG. 2C).

### REFERENCES FOR SUPPLEMENTAL INFORMATION

1. Metzger V T, Lloyd-Smith J O, Weinberger L S (2011) Autonomous targeting of infectious superspreaders using engineered transmissible therapies. PLoS computational biology 7: e1002015.
2. Weinberger L S, Schaffer D V, Arkin A P (2003) Theoretical design of a gene therapy to prevent AIDS but not human immunodeficiency virus type 1 infection. J Virol 77: 10028-10036.
3. Perelson A S, Nelson P W (1999) Mathematical analysis of HIV-1 dynamics in vivo. Siam Review 41: 3-44.
4. Nowak M A, May R M (2000) Virus dynamics: mathematical principles of immunology and virology. Oxford; New York: Oxford University Press. xii, 237 p. p.
5. Josefsson L, King M S, Makitalo B, Brannstrom J, Shao W, et al. (2011) Majority of CD4+ T cells from peripheral blood of HIV-1-infected individuals contain only one HIV DNA molecule. Proc Natl Acad Sci USA 108: 11199-11204.
6. Tsimring L S, Levine H, Kessler D (1996) RNA virus evolution via a fitness-space model. Phys Rev Lett 76: 4440-4443.
7. Rouzine I, Wakeley J, Coffin J (2003) The solitary wave of asexual evolution. Proc

Natl Acad Sci USA 100: 587-592.
8. Rouzine I M, Brunet E, Wilke C O (2008) The traveling-wave approach to asexual evolution: Muller's ratchet and speed of adaptation. Theor Popul Biol 73: 24-46.
9. Desai M M, Fisher D S (2007) Beneficial mutation selection balance and the effect of linkage on positive selection. Genetics 176: 1759-1798.
10. Rouzine I M, Coffin J M (2005) Evolution of human immunodeficiency virus under selection and weak recombination. Genetics 170: 7-18.
11. Rouzine I M, Coffin J M (2007) Highly fit ancestors of a partly sexual haploid population. Theor Popul Biol 71: 239-250.
12. Rouzine I M, Coffin J M (2010) Multi-site adaptation in the presence of infrequent recombination. Theor Popul Biol 77: 189-204.
13. Hallatschek 0 (2010) The noisy edge of traveling waves. Proc Natl Acad Sci USA 108: 1783-1787.
14. Neher R A, Shraiman B I, Fisher D S (2010) Rate of adaptation in large sexual populations. Genetics 184: 467-481.
15. Sergeev R A, Batorsky R E, Rouzine I M (2010) Model with two types of CTL regulation and experiments on CTL dynamics. J Theor Biol 263: 369-384.
16. Kuroda M J, Schmitz J E, Charini W A, Nickerson C E, Lifton M A, et al. (1999) Emergence of CTL coincides with clearance of virus during primary simian immunodeficiency virus infection in rhesus monkeys. J Immunol 162: 5127-5133.
17. Letvin N L, Mascola J R, Sun Y, Gorgone D A, Buzby A P, et al. (2006) Preserved CD4+ central memory T cells and survival in vaccinated SIV-challenged monkeys. Science 312: 1530-1533.
18. Sun Y, Schmitz J E, Buzby A P, Barker B R, Rao S S, et al. (2006) Virus-specific cellular immune correlates of survival in vaccinated monkeys after simian immunodeficiency virus challenge. J Virol 80: 10950-10956.
19. Chen J, Nikolaitchik 0, Singh J, Wright A, Bencsics C E, et al. (2009) High efficiency of HIV-1 genomic RNA packaging and heterozygote formation revealed by single virion analysis. Proc Natl Acad Sci USA 106: 13535-13540.
20. Sergeev R A, Batorsky R E, Coffin J M, Rouzine I M (2010) Interpreting the effect of vaccination on steady state infection in animals challenged with Simian immunodeficiency virus. J Theor Biol 263: 385-392.

### Example 2

### Generation and Characterization of an HIV TIP

Experiments were conducted to assess expression of full-length genomic RNA for TIP relative to HIV-1 within cells (parameter P in models).

293T were co-transfected with HIV-1 NL43G and TIP plasmids by adding of NL4-3G plasmid and 1 μg of TIP plasmid complexed with X-tremeGENE 9 DNA Transfection Reagent (Roche) to 10⁶ cells in each well of a 6 well plate. At 16 hours post transfection, the media was changed, and at 48 hours post transfection virus was harvested by collecting the media above the adherent cells, clarified by centrifugation at 3000 g for 10 minutes, and virus purified by spinning the clarified supernatant through a 20% sucrose cushion for 90 minutes at 25 k rpm in a SW41 rotor. The supernatant was removed and the virus resuspended in a final volume of 350 μl. 275 μl was used for titering on CEM & MT4 cells (obtained from NIH AIDS Reagent Program) and 40 μl was frozen for reverse transcription-quantitative polymerase chain reaction (RT-qPCR). 100 μl of viral supernatant was added to 75,000 MT4 T cells (NIH AIDS Reagent Program) and spinoculation occurred for 2 h, at 4° C. and 1200×g. One timepoint was taken at 18 hours post infection (100 μl). 40 μl of cell culture was analyzed by flow cytometry on a MACSQuant™ VYB cytometer.

For HIV only control: 293T HEK cells were transfected with a plasmid encoding a full-length molecular clone of HIV-1 (HIV NL4-3G) that expresses the full complement of HIV-1 proteins as well as green fluorescent protein (GFP) from the Nef reading frame in a (GFP-IRES-Nef cassette).

For TIP+HIV experiment: 293T HEK cells were co-transfected with HIV N4-3G as well as a plasmid encoding the TIP designated “TIP-SR2-D1”. The TIP vector expresses blue fluorescent protein (mtagBFP2, which can be abbreviated as BFP) from an EF-1a promoter. The TIP vector map and the HIV-1 (NL4-3) map are shown in FIG. 17. A comparison of the TIP (SR2-D1) (SEQ ID NO: 2) and HIV-1 (NL4-3) (SEQ ID NO: 1) nucleotide sequences is presented in FIG. 18.

For RT-qPCR, 2 days post transfection, cells and supernatant were separated, cells were lysed and analyzed using the primer sets detailed below to analyze spliced and un-spliced forms of the HIV and TIP RNA. RT-qPCR results were normalized to both beta-actin (“ActB”) and peptidylprolyl isomerase A PPIA cellular housekeeping genes.

Supernatant was clarified by passage through a 200 μm filter and ultracentrifuged at 20,000×g to concentrate the virus. A 100 μL aliquot of was taken for qPCR analysis to analyze the packaged RNAs using the same primer sets as for the whole cell analysis.

The remainder of the supernatant was used to infect MT-4 T cells (NIH AIDS Reagent Program) and the infections analyzed by flow cytometry for GFP and BFP expressing cells at 4 days post-infection.

The data are depicted in FIGS. 9-12.

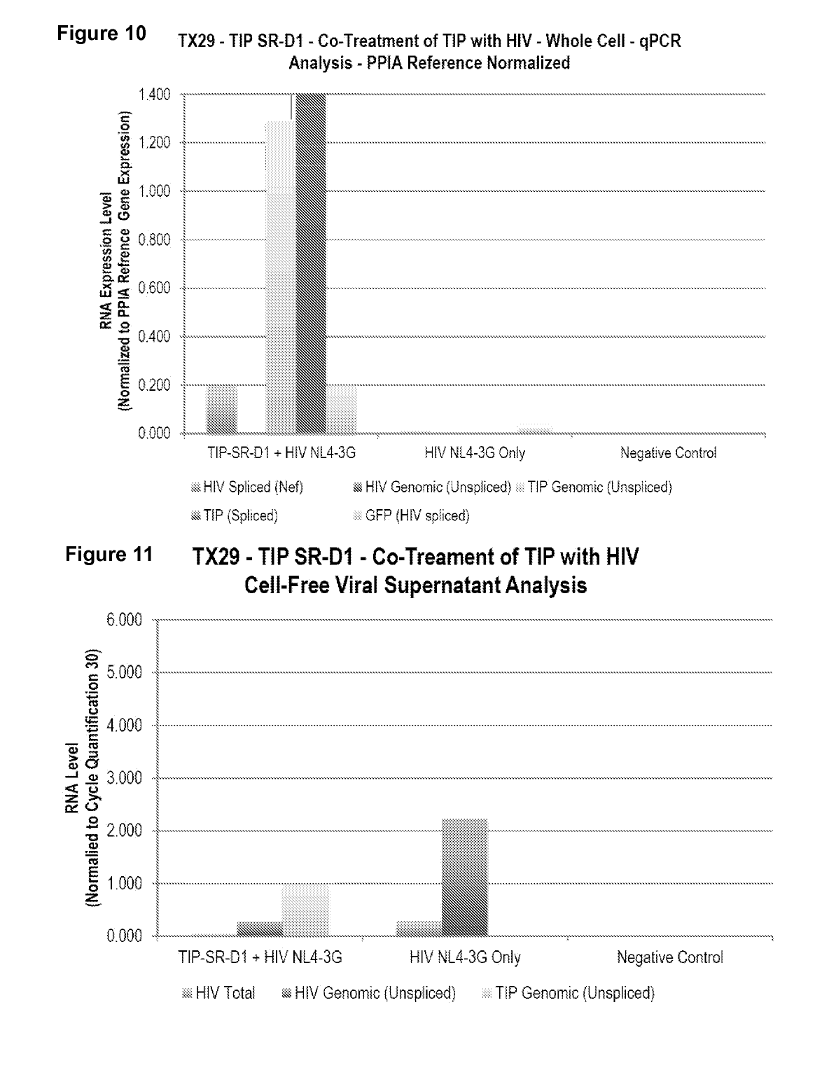

As shown in FIGS. 9 and 10, TIP gRNA (green) is significantly increased compared to HIV-1 gRNA (red) in co-transfected cells: TIP full-length genomic RNA (gRNA) (green) is approximately >100-fold more abundant than HIV-1 gRNA (red) in 293T cells after co-transfection with TIP SR2-D1 and HIV NL4-3G.

As shown in FIGS. 9 and 10, splicing is reduced in TIP compared to HIV-1, generating a greater ratio of gRNA:spliced RNA for TIP compared to HIV-1: the ratio of TIP spliced RNA (purple) to TIP gRNA (green) is ˜8 while HIV gRNA (blue) to HIV-1 gRNA (red) is ˜21 fold.

As shown in FIG. 11, TIP virion RNA (green) is significantly increased compared to HIV-1 virion RNA (red): TIP virion RNA (gRNA) (green) is approximately ˜3.5-fold more abundant than HIV-1 virion RNA (red) in 293T cells after co-transfection with TIP SR2-D1 and HIV NL4-3G. The data presented in FIG. 11 also show that HIV-1 viral gRNA is significantly reduced in presence of TIP (compare red bars, left column and middle column).

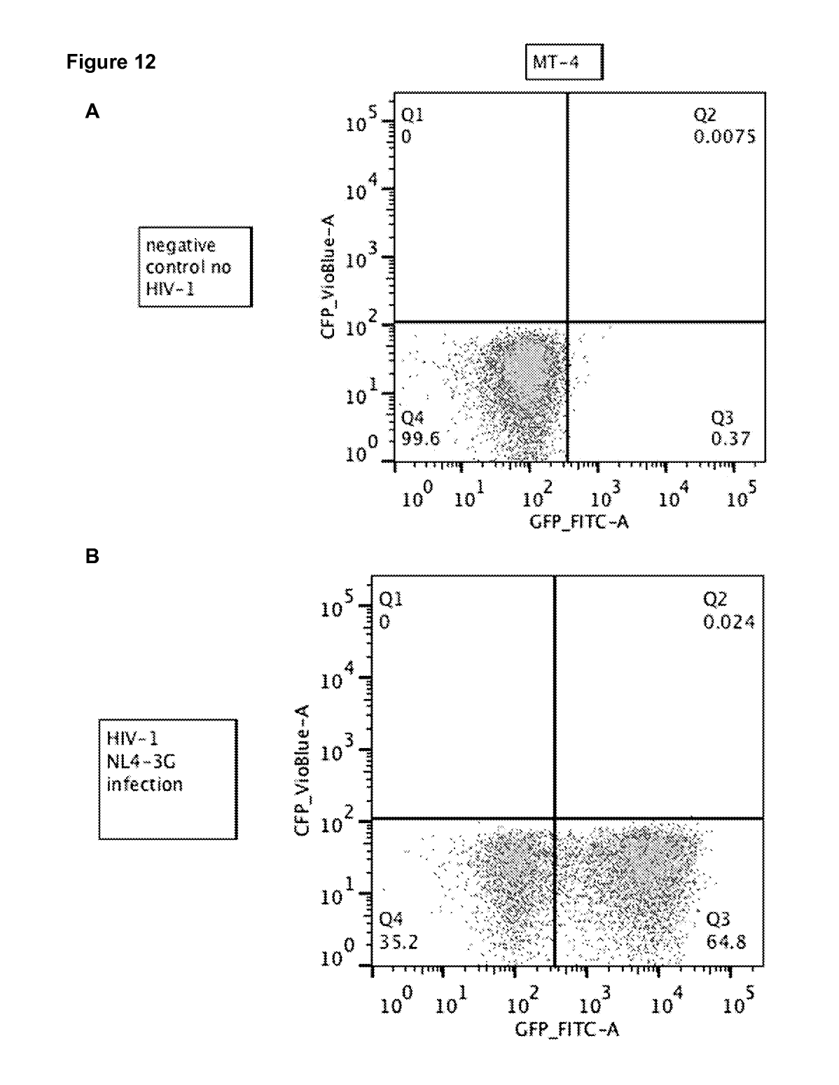

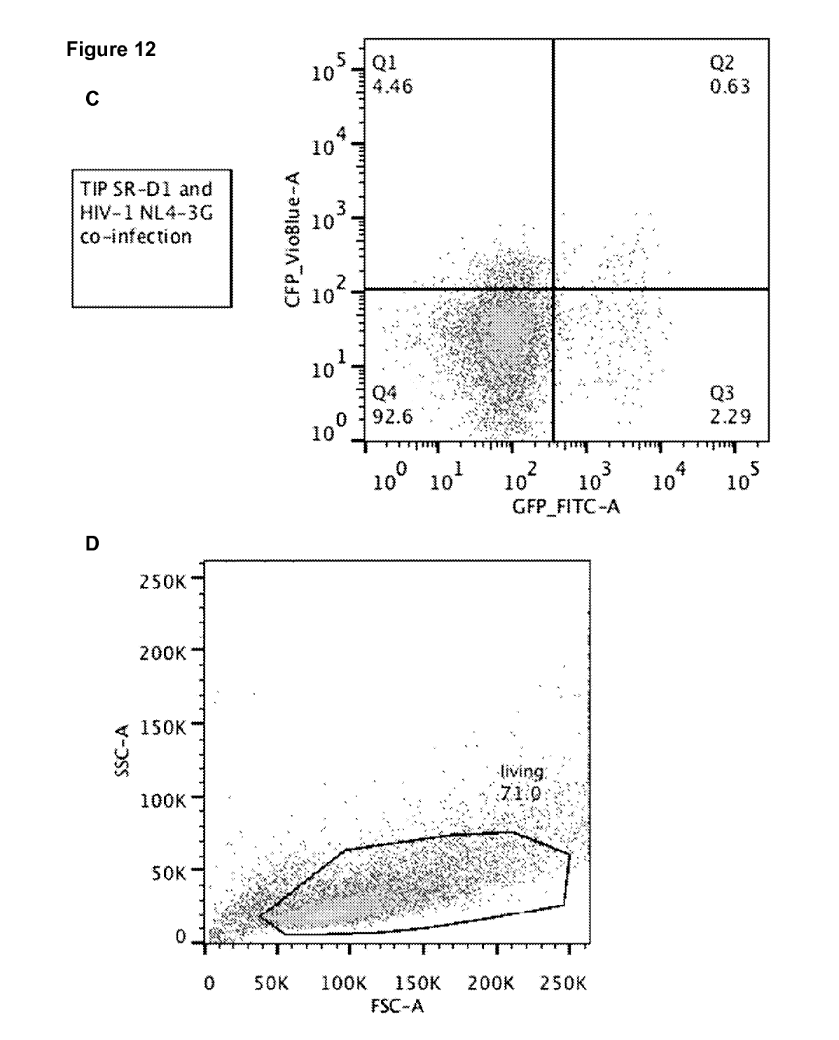

FIG. 12 presents flow cytometry data. As shown in FIG. 12, TIP SR2-D1 is mobilized by HIV-1 and spreads more efficiently than HIV-1 (>5% TIP SR2-D1 spread versus <3% HIV-1 transmission through culture (see bottom graph Q1+Q2>5% versus Q2+Q3<3%). The data presented in FIG. 12 also show that TIP SR2-D1 co-infection significantly interferes with HIV-1 infection (>64% infection in absence of TIP, <3% infection in presence of TIP).

### Example 3

### Generation and Characterization of Additional HIV TIPs

A construct in which the EF1a promoter is deleted from the TIP (SR2-D1) construct was generated and is referred to herein as: “TIP (SR2-D1-delEF1a)” (SEQ ID NO: 18). In addition, a construct in which the EF1a promoter as well as the BFP encoding sequences are deleted from the TIP (SR2-D1) construct was generated and is referred to herein as: “TIP (SR2-D1-delEF1a-delmTagBFP2)” (SEQ ID NO: 19).

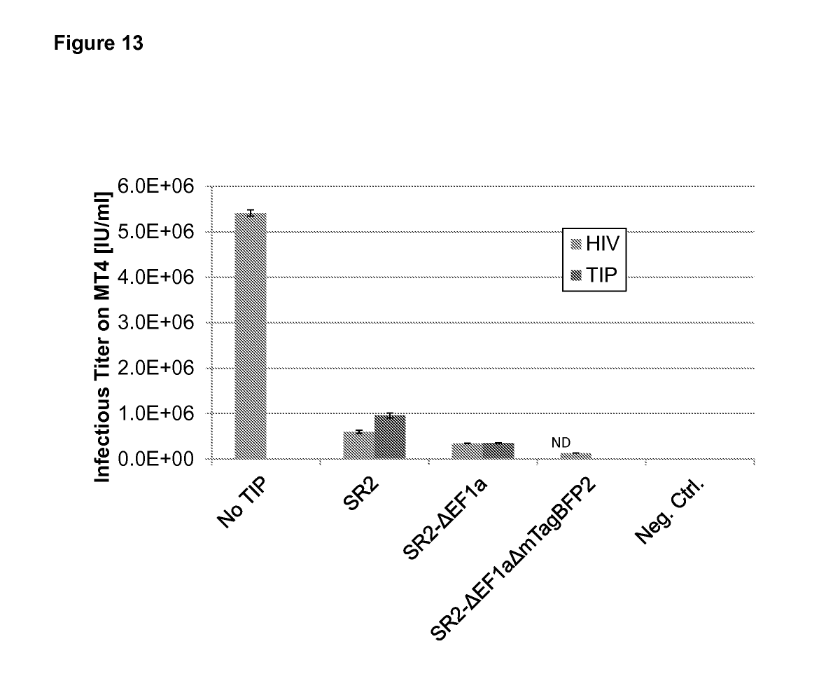

Various constructs were tested and the results are depicted in FIG. 13. Additional experiments were performed to observe the effects of various TIP-SR2 based constructs on the HIV viral load. Since previous experiments showed that the TIP-SR2 construct interferes with HIV viral load, new experiments were performed to determine the dosage sensitivity of TIP constructs co-transfected with HIV (NL4-3G), where the HIV dosage is held constant.

1×10⁶ Naïve HEK-293T cells were plated per well of a 6 well plate. Cells were then co-transfected with 1 μg of NL4-3G and a TIP (TIP-SR2 (SEQ ID NO: 20), TIP-SR2-D1 (SEQ ID NO: 2), TIP-SR2_delEF1a (deletion of EF1a promoter) (SEQ ID NO: 18), or TIP-SR2 delEF1a_delmTagBFP2 (deletion of EF1a promoter and mTagBFP2)(SEQ ID NO: 19). Supernatants were harvested (0.45 uM filtered and concentrated), qPCR lysates were made, and transfected 293T cells were fixed for FACS analysis @ 48 hrs post transfection. 1.5×10⁵ naïve MT4s per well of 96 well plate were treated with the harvested supernatants in a dose range from 4⁰-4⁴ and obtained a FACS and qPCR time-point of the samples 24 hours post infection/transduction.

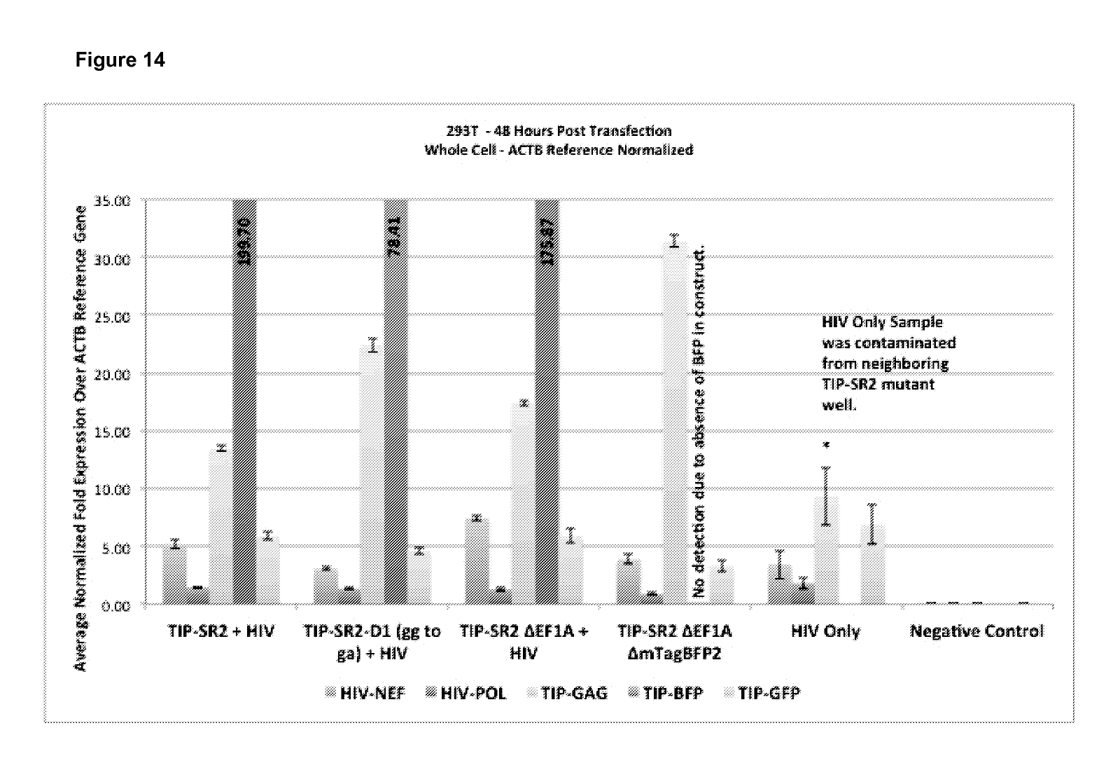

As shown in FIG. 14, deletion of the EF1a promoter showed no change while deletion of EF1a and BFP increased intracellular transcription of TIP in 293T cells. Expression values were normalized to the expression of ACTB reference gene for each sample. The TIP-SR2-D1 construct showed an increase in GAG expression and a proportional decrease in BFP. The deletion of the EF1a promoter showed about a 0.3 fold increase in GAG expression over the parental TIP-SR2 and about a 0.13 fold decrease in BFP expression compared to the parental TIP-SR2. Deleting both the EF1a promoter and the BFP from the TIP-SR2 resulted in about a 2.3 fold increase of GAG specific mRNA. Consistent with the FACS data, there were no significant differences with the intracellular expression of the HIV associated mRNA sequences between the TIP-SR2 mutants and the parental TIP-SR2 construct. In addition, these data confirmed that the TIP-SR2 with the EF1a and mTagBFP2 deletions does not show expression of the BFP region.

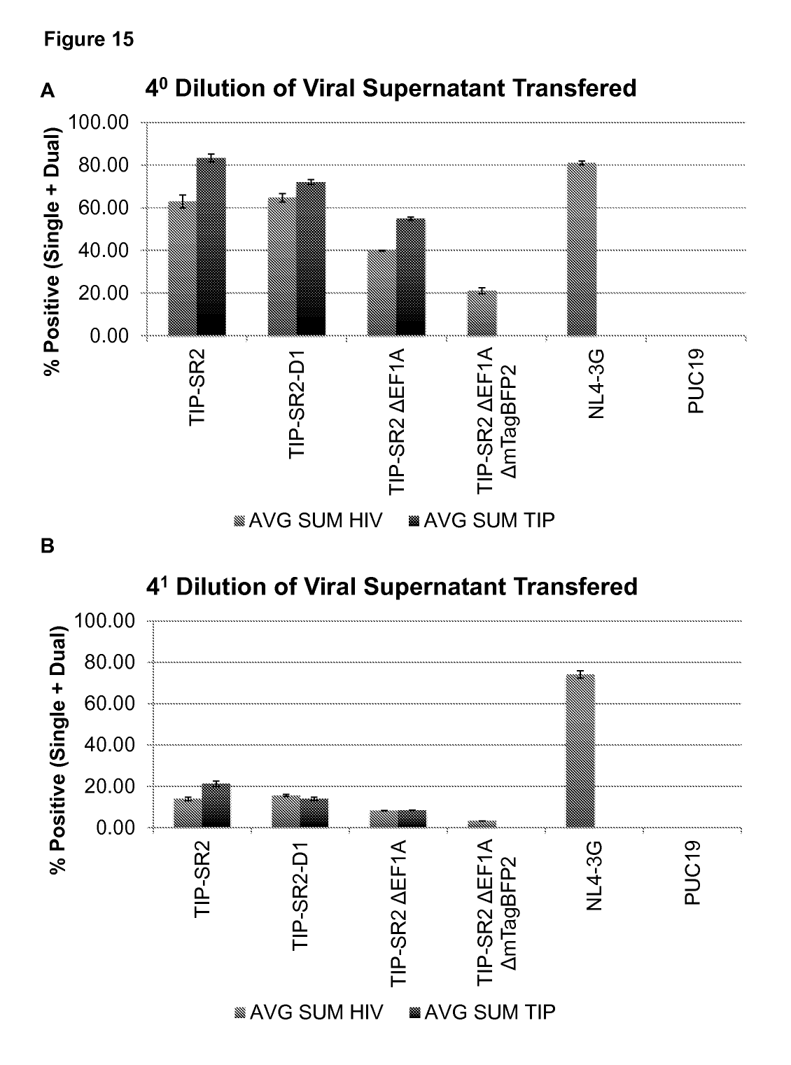

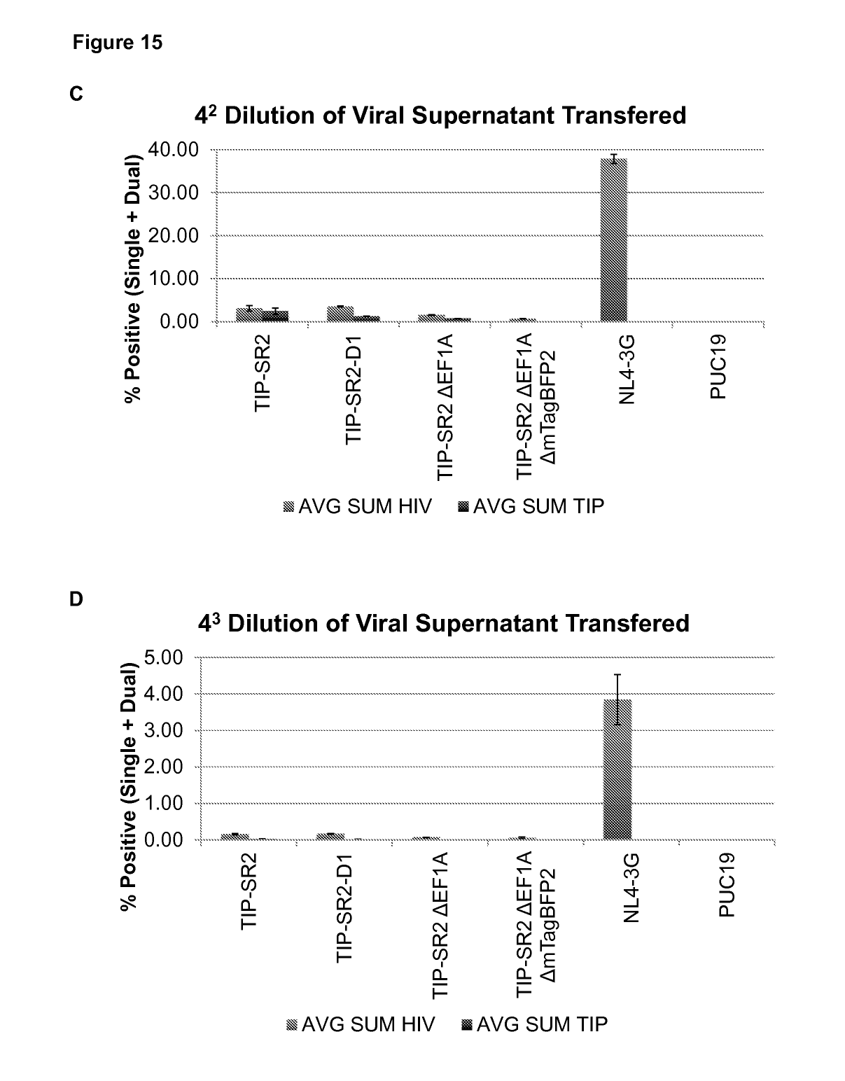

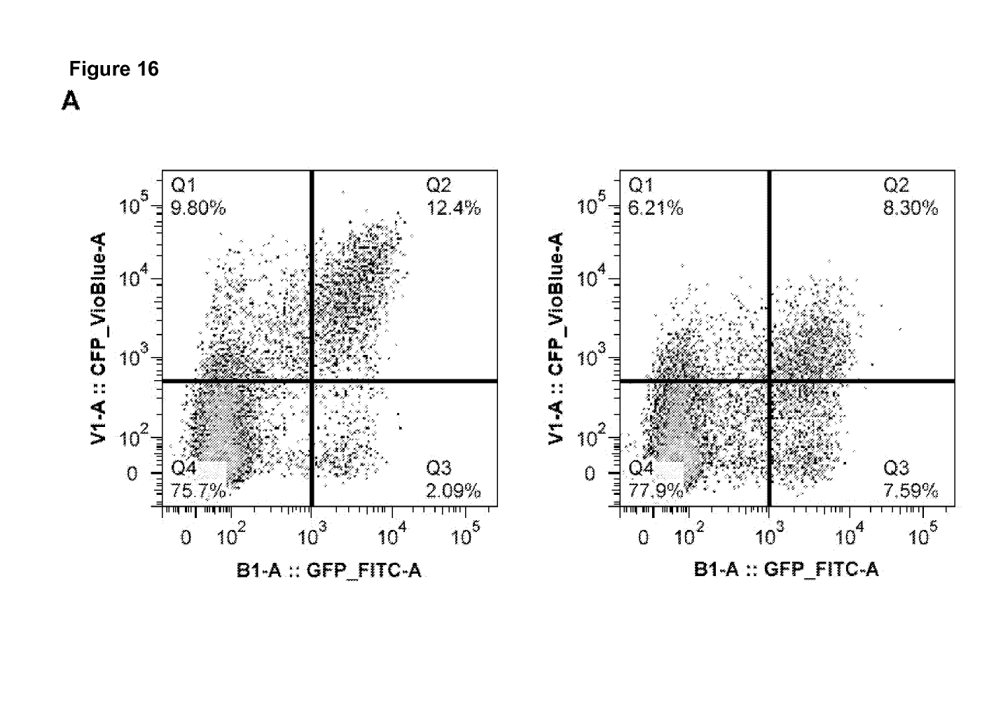

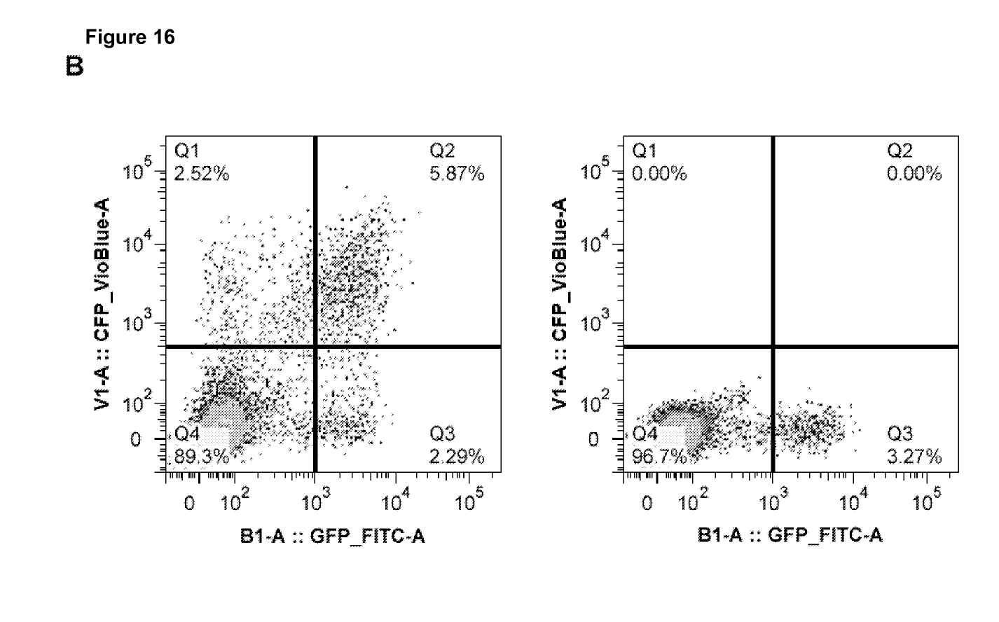

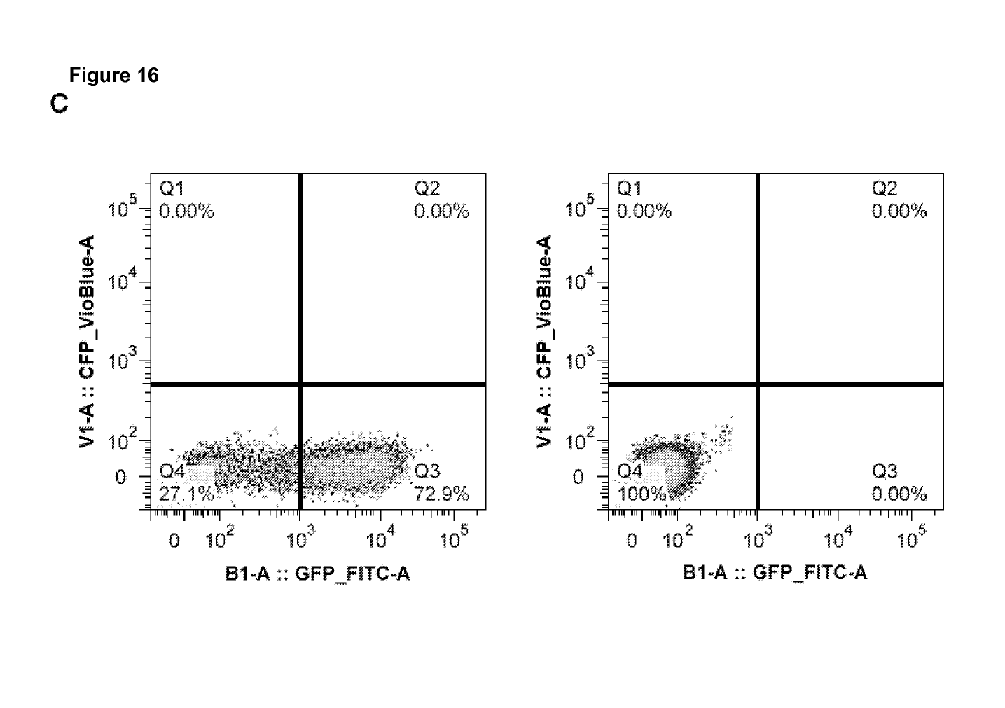

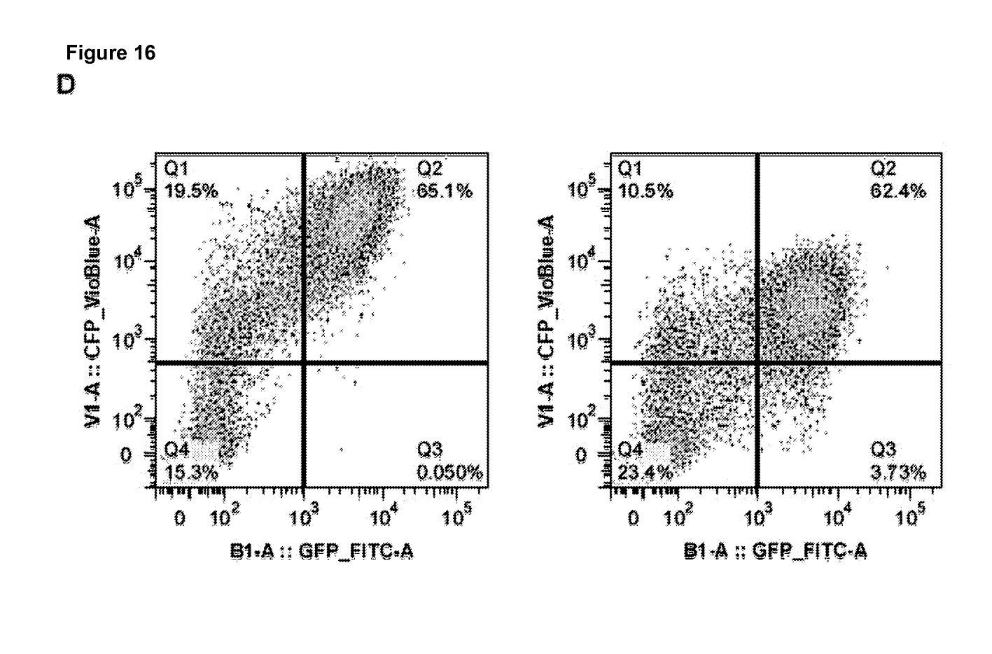

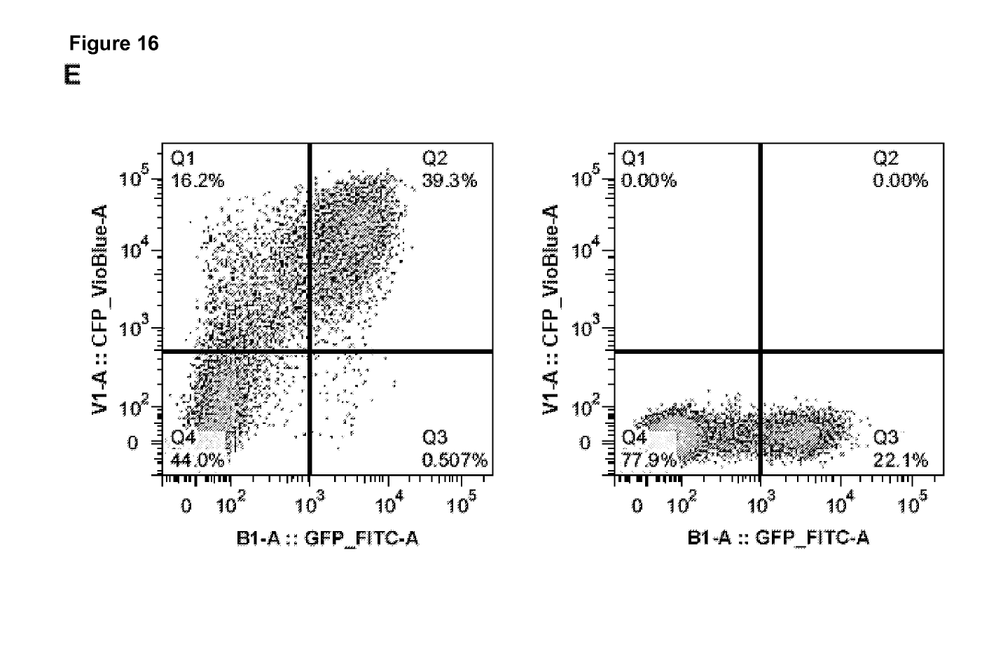

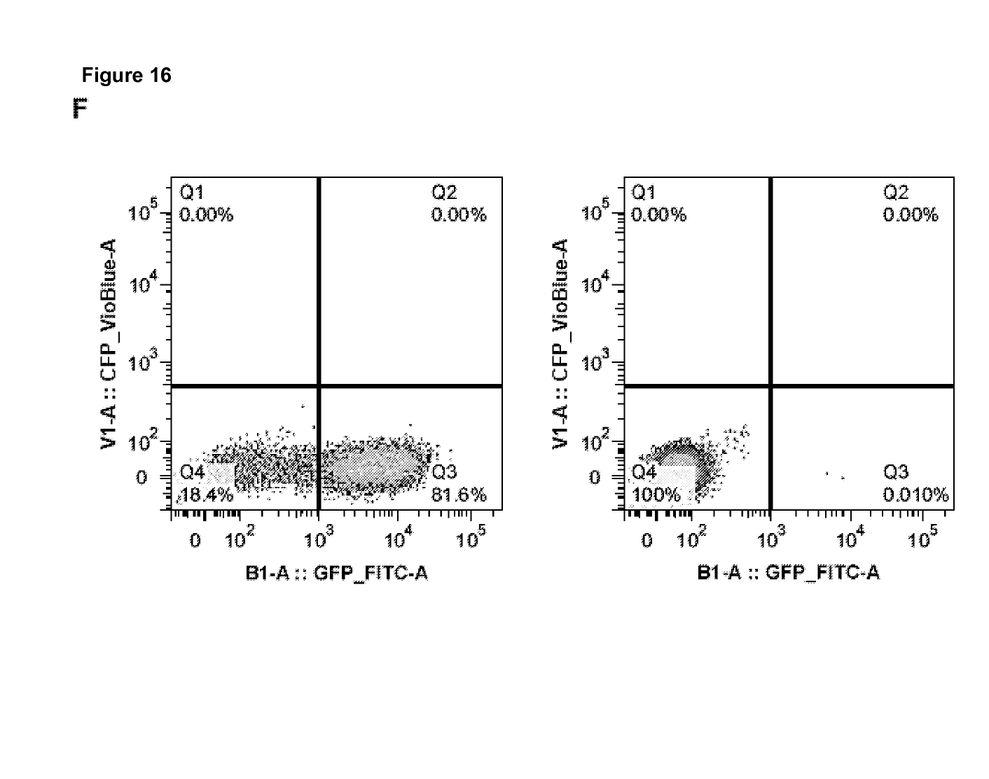

FIGS. 13, 15 and 16 show that supernatant transferred to naïve MT4s showed reduction of HIV positive populations with co-transfection of HIV and TIP compared to wildtype only (FIG. 16 depicts corresponding FACS plots for FIGS. 15 and 13). Consistent with the viral supernatant qPCR data above for the HIV load found with the viral supernatants, the percentage of HIV positive cells was lower in all of the TIP constructs in comparison to NL4-3G-only with the most dramatic being seen with the TIP-SR2 with the delEF1a_delmTagBFP2 deletions. The dilutions used are depicted in each panel of FIG. 15. FIG. 16: A-C: undiluted viral supernatant; D-F: 4¹ dilution of the viral supernatant. FIGS. 16: A and D: left, TIP-SR2 plus NL4-3G; right, TIP-SR2-D1 plus NL4-3G. B and E: left, TIP-SR2-delEF1a plus NL4-3G; right, TIP-SR2-delEF1a-delmTagBFP2 plus NL4-3G. C and F: left, NL4-3G and vector control (PUC19); right, vector only (PUC19).

### Summary of Example 3 Results

293T FACS data show that all of the TIP-SR2 constructs have a similar percentage of TIP positive cells with the exception of the TIP-SR2-D1 that shows 18.42% lower TIP positive than the parental TIP-SR2. Based on the 293T FACS and qPCR data none of the constructs had a direct effect on the transcription or expression of HIV compared to the parental TIP-SR2. Although qPCR data showed that there was an increase in transcription of the GAG associated sequence in intracellular mRNA with the TIP-SR2-delEF1a-delmTagBFP2 construct, the increase was not proportionally found in the viral load found in the 293T supernatants. These data are consistent with data of previous experiments where an increase in the intracellular transcription rate of TIP-SR2 did not have a proportional increase in the packaging or export of TIP RNA into the supernatants.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An interfering, conditionally replicating, human immunodeficiency virus (HIV) construct, the construct comprising:
a) cis-acting elements;
b) an alteration in an HIV nucleotide sequence, wherein the alteration renders any HIV trans element nonfunctional such that the construct is incapable of replication and production of virus on its own but requires replication-competent HIV to act as a helper virus,
wherein genomic RNA encoded by the construct is produced at a higher rate than wild-type HIV when present in a host cell infected with a wild-type HIV,
such that the ratio of construct-encoded gRNA to wild-type HIV gRNA is higher than about 1 in the cell
wherein the construct has a basic reproductive ratio (R₀)>1, and
wherein the construct does not include any heterologous nucleotide sequence that encode a gene product.

2. The construct of claim 1, wherein the HIV trans element is an HIV-encoded encoded protein selected from Env, Gag, Pol, Tat, Rev, Vpr, Nef, Vif, and Vpu.

3. The construct of claim 1, wherein the construct is packaged with a higher efficiency than wild-type HIV when present in a host cell infected with a wild-type HIV.

4. The construct of claim 1, wherein the cis-acting elements include:
i) a Ψ packaging signal;
ii) a lentiviral rev responsive element (rre) sequence;
iii) a lentiviral long terminal repeat; and
iv) at least one cis element embedded within an HIV protein-coding sequence.

5. The construct of claim 1, wherein the alteration comprises a deletion in an HIV splice donor.

6. The construct of claim 1, wherein the alteration comprises a deletion in an HIV splice acceptor.

7. The construct of claim 1, wherein the construct comprises a deletion of the nucleotide sequence encoding HIV Nef.

8. A particle comprising:
a) the construct of claim 1;
b) a viral envelope protein.

9. The particle of claim 8, wherein the envelope protein comprises gp120.

10. The particle of claim 8, wherein the envelope protein is a non-HIV protein.

11. A pharmaceutical formulation comprising:
a) a particle comprising the construct according to claim 1;
b) a pharmaceutically acceptable excipient.

12. A package for use in delivering the construct of claim 1 to an individual, the package comprising a container comprising the formulation of claim 11.

13. The package of claim 10, wherein the container is a syringe.

14. A method of reducing human immunodeficiency virus viral load in an individual, the method comprising administering to the individual an effective amount of a pharmaceutical formulation of claim 11.

15. The method of claim 14, further comprising administering to the individual an effective amount of an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

16. The method of claim 14, wherein the individual has been diagnosed with an HIV infection.

17. The method of claim 14, wherein the individual is considered to be at higher risk than the general population of becoming infected with HIV.

18. The method of claim 14, further comprising administering to the individual an effective amount of an agent that reactivates reactivating latent HIV integrated into the genome of a cell infected with HIV.

19. A biological fluid comprising the construct of claim 1 or a derivative thereof.

20. The biological fluid of claim 17, wherein the biological fluid is plasma.

21. A method of generating a variant interfering, conditionally replicating, human immunodeficiency virus (HIV) construct, the method comprising:
a) introducing the construct of claim 1 into a first individual;
b) obtaining a biological sample from a second individual to whom the construct of claim 1 has been transmitted from the first individual, wherein the construct present in the second individual is a variant of the construct of claim 1;
c) cloning the variant construct from the second individual.


SEQUENCE LISTING (20 sequences)

SEQ ID NO: 1 - DNA, 9709 bp, human immunodeficiency virus
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggctgcacgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 4669 bp, Artificial sequence
  synthetic nucleic acid sequence
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgatgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcgaaaatggcagtattcatccaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgctactagctcgagaagaattcaaggcgcgccaagctagcaagttaacaaatcgataagccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaacctgcaggtttgtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 3 - RNA, 40 nt, Human immunodeficiency virus
ggacucggcuugcugaagygcrcwcrgcaagaggcgagrg

SEQ ID NO: 4 - RNA, 19 nt, Human immunodeficiency virus
rgcgacuggugaguacgch

SEQ ID NO: 5 - RNA, 23 nt, Human immunodeficiency virus
ugacuagcggaggcuagaaggag

SEQ ID NO: 6 - RNA, 20 nt, Human immunodeficiency virus
ugggugcgagagcgucarua

SEQ ID NO: 7 - DNA, 19 bp, Human immunodeficiency virus
ggcgactggtgagtacgcc

SEQ ID NO: 8 - DNA, 19 bp, Human immunodeficiency virus
ggcggctggtgagtacgcc

SEQ ID NO: 9 - DNA, 19 bp, Human immunodeficiency virus
ggcgaatggtgagtacgcc

SEQ ID NO: 10 - DNA, 19 bp, Human immunodeficiency virus
agcgactggtgagtacgct

SEQ ID NO: 11 - DNA, 19 bp, Human immunodeficiency virus
agcgaccggtgagtacgct

SEQ ID NO: 12 - RNA, 11 nt, Human immunodeficiency virus
aaggugaaggg

SEQ ID NO: 13 - RNA, 11 nt, Human immunodeficiency virus
aagguagguca

SEQ ID NO: 14 - RNA, 11 nt, Human immunodeficiency virus
cuagacuagag

SEQ ID NO: 15 - RNA, 11 nt, Human immunodeficiency virus
gcaguaaguag

SEQ ID NO: 16 - DNA, 77 bp, Human immunodeficiency virus
ataacaaaagccttaggcatctcctatggcaggaagaagagagttaggcagggatattcaccattatcgtttcagcc

SEQ ID NO: 17 - protein, 36 aa, Artificial sequence
  synthetic amino acid sequence
YTSLIHSLIEESQNQQEKNEQELLELDKWASLWNWF

SEQ ID NO: 18 - DNA, 3558 bp, Artificial Sequence
  synthetic nucleic acid sequence
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgatgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcgaaaatggcagtattcatccaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattggctccggtgcccgtcagtgggcagagcgcacatcgcccacagtccccgagaagttggggggaggggtcggcaacgctactagctcgagaagaattcaaggcgcgccaagctagcaagttaacaaatcgataagccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaacctgcaggtttgtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 19 - DNA, 2791 bp, Artificial Sequence
  synthetic nucleic acid sequence
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactgatgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcgaaaatggcagtattcatccaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattggctccggtgcccgtcagtgggcagagcgcacatcgcccacagtccccgagaagttggggggaggggtcggcaacctgcaggtttgtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 20 - DNA, 4669 bp, Artificial Sequence
  synthetic nucleic acid sequence
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcgaaaatggcagtattcatccaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgctactagctcgagaagaattcaaggcgcgccaagctagcaagttaacaaatcgataagccaccatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaacctgcaggtttgtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca